(19) United States
(12) Patent Application Publication
(10) Pub. No.: US 2014/0073008 A1
(43) Pub. Date: Mar. 13, 2014

(54) GENE CLUSTER FOR BIOSYNTHESIS OF CYCLOCLAVINE

(71) Applicants: Hartwig Schroeder, Nussloch (DE); Birgit Hoff, Pfungstadt (DE)

(72) Inventors: Hartwig Schroeder, Nussloch (DE); Birgit Hoff, Pfungstadt (DE)

(73) Assignee: BASF SE, Ludwigshafen (DE)

(21) Appl. No.: 14/000,080

(22) PCT Filed: Feb. 24, 2012
(86) PCT No.: PCT/EP2012/053197

§ 371 Date: Nov. 22, 2013

Related U.S. Application Data
(60) Provisional application No. 61/448,691, filed on Mar. 3, 2011.

(30) Foreign Application Priority Data
Mar. 3, 2011 (EP) 11156853.1

(51) Int. Cl. C12P 17/18

(52) U.S. Cl. CPC C12P 17/182
USPC 435/69.1; 536/23.2; 435/320.1; 435/252.3; 435/254.11; 435/254.2; 435/419; 435/257.2; 530/350; 530/387.9; 435/119; 536/23.74; 435/193; 435/190

(57) ABSTRACT

The invention in principle pertains to the field of recombinant manufacture of alkaloids and, in particular, cycloclavine. It provides polynucleotides for the gene cluster of enzymes involved in the biosynthesis of cycloclavin as well as vectors and host cells comprising such polynucleotides. Also provided are polypeptides encoded by the said polynucleotides which can be applied in a method for the manufacture of cycloclavine also encompassed by the present invention.

The invention in principle pertains to the field of recombinant manufacture of alkaloids and, in particular, cycloclavine. It provides polynucleotides for the gene cluster of enzymes involved in the biosynthesis of cycloclavin as well as vectors and host cells comprising such polynucleotides. Also provided are polypeptides encoded by the said polynucleotides which can be applied in a method for the manufacture of cycloclavine also encompassed by the present invention.

Prenylated indole alkaloids to which cycloclavine is belonging are hybrid natural products derived from prenyl diphosphates and tryptophan or its precursors and widely distributed in filamentous fungi, especially in the genera Penicillium and Aspergillus of ascomycota. These compounds represent a group of natural products with diverse chemical structures and biological activities. Significant progress on their biosynthesis has been achieved in recent years by identification of biosynthetic gene clusters from genome sequences and by molecular biological and biochemical investigations. In addition, a series of prenylated indole derivatives have been produced by chemoenzymatic synthesis using overproduced and purified enzymes. They are widely distributed in terrestrial and marine organisms, especially in the genera Penicillium and Aspergillus of ascomycota, and display broad structure diversity. These compounds often carry biological and pharmacological activities distinct from their non-prenylated aromatic precursors.

Ergot alkaloids are a complex family of indole derivatives with diverse structures and biological activities (Flieger 1997, Folia Microbiol (Praha) 42:3-30; Schardl 2006, Chem Biol 63:45-86). They are produced by fungi of two orders and plants of three families. The important producers are fungi of the genera Claviceps, Penicillium, and Aspergillus (Flieger 1997, loc cit.; Schradl loc cit.). Both the natural ergot alkaloids and their semisynthetic derivatives are in widespread use in modern medicine and exhibit a broad spectrum of pharmacological activities, including uterotonic activity, modulation of blood pressure, control of the secretion of pituitary hormones, migraine prevention, and dopaminergic and neuroleptic activities (de Groot 1998, Drugs 56:523-535; Haarmann 2009, Mol Plant Pathol 10:563-577; Schardl 2006, loc cit.) Ergot alkaloids can be divided into two classes according to their structural features, i.e., amide derivatives of D-lysergic acid and the clavine alkaloids (Flieger 1997, loc cit.) Gröger 1998, Alkaloids Chem Biol 50:171-218).

The members of the first group are usually composed of lysergic acid and a peptide moiety, e.g., ergotamine. The clavines like cyloclavine, agroclavine and fumigaclavines or their precursors like chanoclavine-I and its aldehyde merely consist of a tricyclic or tetracyclic ring system lacking a peptide moiety. The clavine-type alkaloids fumigaclavines are produced by Penicillium and Aspergillus, e.g., A. fumigatus (Flieger 1997, loc cit.), but not by the fungal family of the Clavicipitaceae, e.g., C. purpurea (Flieger 1997, loccit) Conversely, ergopeptines are produced by C. purpurea, but not by A. fumigatus. Comparison of the precursors indicated that the early stages of their biosynthetic pathway are very likely shared by A. fumigatus and C. purpurea, whereas later steps in the pathway differ in the two fungal taxa (Li 2006, Chembiochem 7:158-164; Panaccione 2005, FEMS Microbiol Lett 251:9-17; Schardl 2006, loc cit.).

With the help of bioinformatic approaches, a biosynthetic gene cluster of fumigaclavine C has been identified in the genome of A. fumigatus Af293 and A1163 (Li 2006, Chembiochem 7; Steffan 2009, Curr Med Chem 16:218-231; Unsöld 2005, Microbiology 151:1499-1505). The discovery of this cluster provides a convenient way to identify candidate genes for the early stages of the biosynthesis of ergot alkaloids by comparison of the clusters in C. purpurea (Tudzynski 1999, Mol Gen Genet 261:133-141) and A. fumigatus. Seven orthologous genes are found in the biosynthetic gene clusters of fumigaclavines in A. fumigatus and of ergot alkaloids in C. purpurea (Panaccione 2005, FEMS Microbiol Lett 251:9-17; Unsöld 2005, Microbiology 151:1499-1505). These seven genes are proposed to encode enzymes that catalyze the common steps in the biosynthesis of fumigaclavines in A. fumigatus and of ergot alkaloids in C. purpurea (Unsöld 2006, Chembiochem 7:158-164). Until now, only two genes involved in the early steps of the ergot alkaloid biosynthesis i.e., dmaW from Claviceps fusiformis and A. fumigatus (termed also fgaPT2) as well as fgaMT from A. fumigatus, have been functionally identified (Coyle 2005, FEMS Microbiol Lett 251:9-17; Unsöld 2005, Microbiology 151:1499-1505). DmaW and FgaPT2 catalyze the prenylation of L-tryptophan at position C4 resulting in the formation of 4-dimethylallyltryptophan ((S)-4-(3-methyl-2-butenyl)-tryptophan, 4-DMAT), which is then converted to 4-Dimethylallyl-L-abrine (4-DMA-L-abrine) by the N-methyltransferase FgaMT (Rigbers 2008, J Biol Chem 283:26859-26868). Feeding experiments in Claviceps sp. cultures with isotope-labeled precursors in the 1970s and 1980s indicated that chanoclavine-I and its aldehyde are intermediates in the conversion of 4-DMA-L-abrine to agroclavine (Floss 1974, J Am Chem Soc 96:1898-1909; Hassam 1981, J Nat Prod 44:756-758). However, an enzymatic conversion of chanoclavine-I to chanoclavine-I aldehyde has not yet been demonstrated by an overproduced and purified enzyme or by crude enzyme extracts from a producer. The direct conversion of chanoclavine-I to agroclavine by crude extracts of an ergot alkaloid producer (Sajdl 1975, Folia Microbiol (Praha) 20:365-367) has been interpreted as the sum of catalytic results of at least three distinct enzymes (Schardl loc cit.). Conversion of chanoclavine-I to chanoclavine-I aldehyde is involved in the biosynthesis of fumigaclavines in A. fumigatus and of ergopeptines in C. purpurea.

Inspection of the remained Wve genes with unknown functions shared by the clusters mentioned earlier revealed one candidate gene for this reaction, i.e., fgaOx2 in A. fumigatus Af293 (=AFUA_{—}2G18000 in GenBank) and cpox2 (easD) in C. purpurea. This gene has been proposed to be a short-chain alcohol dehydrogenase/reductase (SDR) (Schardl loc cit.; Unsöld loc cit.). Indeed, inspection of the sequence of the deduced product of fgaDH revealed the presence of the typical cofactor-binding motif TGxxx[AG]xG (TGGASGIG of amino acids 12-19) and the active site motif YxxxK (YGTSK of amino acids 166-170) of classical SDR (Kavanagh 2008, Cell Mol Life Sci 65:3895-3906). However, neither a functional proof nor a suggestion on its role in the biosynthesis of ergot alkaloids was found in the literature. An orthologous gene, AFUB_{—}033690, has also been identi-Wed in A. fumigatus A1163. fgaDH from A. fumigatus Af293 consists probably of two exons of 589 and 197 bp, respectively, interrupted by an intron of 67 bp.

Isolation of cycloclavine from the seeds of Ipomea hildebrandtii was published as early as 1969 by the Sandoz research group (Stauffacher 1969, Tetrahedron, 25(24), 5879-87). Structure elucidation, including relative and absolute configurations of the stereogenic carbon atoms was also carried out by X-ray analysis of its methobromide derivative. The absolute configuration of the molecule was, however, incorrectly published as 5R,8R,10R instead of the proper one (5R,8S,10S), which corresponds to the structure given by the Sandoz group. Later, other research groups 3 found cycloclavine among other ergot alkaloids in different Aspergillus and Argyreia fungi. Up to now, all clavine-type alkaloids have already been prepared 4 by total synthesis except cycloclavine. Although no significant biological activities have been found among clavine-type alkaloids so far, the total synthesis of this characteristic ring system, in which the ergoline skeletal carbon atoms 8, 9, and 10 form a cyclopropane ring in place of the 9,10 double bond, presents a considerable challenge.

The last so far not yet synthesized member of the Clavine alkaloid family, cycloclavine has been prepd. Starting from 4-bromo-Uhle's ketone via an alkylation step using Et 3-methylamino-propionate followed by intramol. aldol condensation, transformation of the ester group into Me group, finally cyclopropanation of the 8,9 double bond resulted in a six step total synthesis of racemic cycloclavine. Cycloclavine shall be an interesting starting material for developing further LSD-type pharmaceutically active psychomimetic drugs of the alkaloid family. However, the biosynthetic pathway to Cycloclavine has not been elucidated.

The technical problem underlying the present invention can be seen as the provision of means and methods for the recombinant manufacture of cycloclavine or a precursor thereof. The technical problem is solved by the embodiments characterized in the claims and herein below.

The present invention, thus, relates to a polynucleotide comprising one or more nucleic acid sequences selected from the group consisting of:
a) a nucleic acid sequence having a nucleotide sequence as shown in SEQ ID NO: 1, 3, 5, 7, 9, 11, 13, 15, or 17;
b) a nucleic acid sequence encoding a polypeptide having an amino acid sequence as shown in SEQ ID NOs: 2, 4, 6, 8, 10, 12, 14, 16, or 18;
c) a nucleic acid sequence being at least 70% identical to the nucleic acid sequence as shown in SEQ ID NO: 1, 3, 5, 7, 9, 11, 13, 15, or 17 or a nucleic acid sequence being at least 80% identical to the nucleic acid sequence shown in SEQ ID NO: 5, wherein said nucleic acid sequence encodes a polypeptide being involved in cycloclavine synthesis;
d) a nucleic acid sequence encoding a polypeptide being involved in cycloclavine synthesis and having an amino acid sequence which is at least 70% identical to the amino acid sequence as shown in SEQ ID NOs: 2, 4, 8, 10, 12, 14, 16, or 18 or at least 80% identical to the amino acid sequence shown in SEQ ID NO: 6; and
e) a nucleic acid sequence which is capable of hybridizing under stringent conditions to any one of a) to d), wherein said nucleic acid sequence encodes a polypeptide being involved in cycloclavine synthesis.

The term “polynucleotide” as used in accordance with the present invention relates to a polynucleotide comprising a nucleic acid sequence which encodes a polypeptide being involved in the cycloclavine synthesis. As used herein, cycloclavine synthesis encompasses all steps of the biosynthesis of cycloclavine. Accordingly, a polypeptide which is involved in the synthesis of cycloclavine may either convert a substrate into cycloclavine or any of the precursors which occur in the cycloclavine biosynthesis. Details on said synthesis of cycloclavine and the catalytic activities required therefor are disclosed elsewhere herein in detail. Preferably, the polypeptide encoded by the polynucleotide of the present invention shall be capable of increasing the amount of cycloclavine or a precursor thereof upon expression in an organism, preferably a host cell as specified elsewhere herein. Such an increase is, preferably, statistically significant when compared to a control organism which lacks expression of the polynucleotide of the present invention. Whether an increase is significant can be determined by statistical tests well known in the art including, e.g., Student's t-test. More preferably, the increase is an increase of the amount of cycloclavine or a precursor thereof of at least 5%, at least 10%, at least 15%, at least 20% or at least 30% compared to said control. Suitable assays for measuring the amount of cycloclavine or a precursor thereof are described in the accompanying Examples.

As set forth above, the term “cycloclavine synthesis” as used herein encompasses all steps of the biosynthesis of cycloclavine. The steps required for cycloclavine synthesis starting from the amino acid tryptophan are, preferably, (i) conversion of tryptophan into DMAT (4-dimethylallyltryptophan ((S)-4-(3-methyl-2-butenyl)-tryptophan), preferably, catalyzed by a DMAT synthase using DMA-PPi, (ii) conversion of DMAT into methyl-DMAT, preferably, catalyzed by a DMAT methyltransferase, (iii) conversion of methyl-DMAT into chanoclavine I, preferably, catalyzed by a chanoclavine synthase, (iv) conversion of chanoclavine I into chanoclavine aldehyde, preferably, catalyzed by a chanoclavine I dehydrogenase, (v) conversion of chanoclavine aldehyde into agroclavine or festuclavine, preferably, catalyzed by a chanoclavine cyclise and an old yellow enzyme, and (vi) conversion of agroclavine or festuclavine into cycloclavine. A precursor of cycloclavine as referred to in accordance with the present invention, thus, is selected from the group consisting of: DMAT, Methyl-DMAT, Chanoclavine I, Chanoclavine aldehyde, agroclavine and festuclavine.

More preferably, polynucleotides having a nucleic acid sequence as shown in SEQ ID NOs:9 encoding polypeptides having amino acid sequences as shown in SEQ ID NOs: 10 or variants thereof carry out conversion of tryptophan into DMAT and, preferably, exhibit DMAT synthase activity (DmaW).

Polynucleotides having a nucleic acid sequence as shown in SEQ ID NOs: 15 encoding polypeptides having amino acid sequences as shown in SEQ ID NOs: 16 or variants thereof carry out conversion of DMAT into methyl-DMAT and, preferably, exhibit DMAT methyltransferse activity (EasF).

Polynucleotides having a nucleic acid sequence as shown in SEQ ID NOs: 11 encoding polypeptides having amino acid sequences as shown in SEQ ID NOs: 12 or variants thereof carry out conversion of methyl-DMAT into chanoclavine I and, preferably, exhibit chanoclavine synthase activity

Polynucleotides having a nucleic acid sequence as shown in SEQ ID NOs: 5 encoding polypeptides having amino acid sequences as shown in SEQ ID NOs: 6 or variants thereof carry out conversion of chanoclavine I into chanoclavine aldehyde and, preferably, exhibit chanoclavine I dehydrogenase activity.

Polynucleotides having a nucleic acid sequence as shown in SEQ ID NOs: 3 encoding polypeptides having amino acid sequences as shown in SEQ ID NOs: 4 or variants thereof carry out conversion of conversion of chanoclavine aldehyde into agroclavine and or festuclavine, preferably, exhibit chanoclavine festuclavine cyclase activity.

Polynucleotides having a nucleic acid sequence as shown in SEQ ID NOs: 7 encoding polypeptides having amino acid sequences as shown in SEQ ID NOs: 8 or variants thereof carry out conversion of agroclavine or festuclavine into cycloclavine and, preferably, exhibit cycloclavine synthase activity.

Polynucleotides having a nucleic acid sequence as shown in SEQ ID NOs: 1 encoding polypeptides having amino acid sequences as shown in SEQ ID NOs: 2 or variants thereof carry out roles in the pathway of agroclavine or festuclavine or cycloclavine and, preferably, exhibit regulation activity.

Polynucleotides having a nucleic acid sequence as shown in SEQ ID NOs: 13 encoding polypeptides having amino acid sequences as shown in SEQ ID NOs: 14 or variants thereof carry out conversion of agroclavine or festuclavine into cycloclavine and, preferably, exhibit cycloclavine synthase activity.

A polynucleotide encoding a being involved in cycloclavine synthesis as specified above has been obtained in accordance with the present invention, preferably, from Botryotinia fuckeliana. However, orthologs, paralogs or other homologs may be identified from other species. Preferably, they are obtained from fungi such as Aspergilli species, Claviceps species, or Penicillium species.

Thus, the term “polynucleotide” as used in accordance with the present invention further encompasses variants of the aforementioned specific polynucleotides representing orthologs, paralogs or other homologs of the polynucleotide of the present invention. Moreover, variants of the polynucleotide of the present invention also include artificially generated muteins. Said muteins include, e.g., enzymes which are generated by mutagenesis techniques and which exhibit improved or altered substrate specificity, or codon optimized polynucleotides. The polynucleotide variants, preferably, comprise a nucleic acid sequence characterized in that the sequence can be derived from the aforementioned specific nucleic acid sequences shown in any one of SEQ ID NOs: 1, 3, 5, 7, 9, 11, 13, 15 or 17 by a polynucleotide encoding a polypeptide having an amino acid sequence as shown in any one of SEQ ID NOs: 2, 4, 6, 8, 10, 12, 14, 16 or 18 by at least one nucleotide substitution, addition and/or deletion, whereby the variant nucleic acid sequence shall still encode a polypeptide be involved in cycloclavine synthesis and, in particular has a activity as specified above for the corresponding lead sequence (indicated in the SEQ ID Nos) from which the variant has been derived. Preferably, an amino acid substitution envisaged in a polypeptide encoded by a variant polynucleotide in accordance with the present invention is a modification which has no or little effect on activity. Such a conservative substitution signifies that one amino acid residue or a plurality of amino acid residues has been replaced with a chemically analogous but different amino acid residue in such a way that the activity of the polypeptide is essentially unchanged. The case where a certain hydrophobic amino acid residue is replaced with a different hydrophobic amino acid residue and the case where a certain polar amino acid residue is replaced with a different polar amino acid residue can be cited as examples. Functionally analogous amino acids with which such substitutions can be carried out are known in this field of technology as similar amino acids. Practical examples include alanine, valine, isoleucine, leucine, proline, tryptofan, phenylalanine, methionine and the like as non-polar (hydrophobic) amino acids and glycine, serine, threonine, tyrosine, glutamine, asparagine, cystine and the like as polar (neutral) amino acids. Arginine, histidine, lycine and the like can be cited as (basic) amino acids which have a positive electrical charge, and aspartic acid, glutamic acid and the like can be cited as (acidic) amino acids which have a negative electrical charge. Variants also encompass polynucleotides comprising a nucleic acid sequence which is capable of hybridizing to the aforementioned specific nucleic acid sequences, preferably, under stringent hybridization conditions. These stringent conditions are known to the skilled worker and can be found in Current Protocols in Molecular Biology, John Wiley & Sons, N.Y. (1989), 6.3.1-6.3.6. A preferred example for stringent hybridization conditions are hybridization conditions in 6× sodium chloride/sodium citrate (=SSC) at approximately 45° C., followed by one or more wash steps in 0.2×SSC, 0.1% SDS at 50 to 65° C. The skilled worker knows that these hybridization conditions differ depending on the type of nucleic acid and, for example when organic solvents are present, with regard to the temperature and concentration of the buffer. For example, under “standard hybridization conditions” the temperature differs depending on the type of nucleic acid between 42° C. and 58° C. in aqueous buffer with a concentration of 0.1 to 5×SSC (pH 7.2). If organic solvent is present in the abovementioned buffer, for example 50% formamide, the temperature under standard conditions is approximately 42° C. The hybridization conditions for DNA: DNA hybrids are, preferably, 0.1×SSC and 20° C. to 45° C., preferably between 30° C. and 45° C. The hybridization conditions for DNA:RNA hybrids are, preferably, 0.1×SSC and 30° C. to 55° C., preferably between 45° C. and 55° C. The abovementioned hybridization temperatures are determined for example for a nucleic acid with approximately 100 bp (=base pairs) in length and a G+C content of 50% in the absence of formamide. The skilled worker knows how to determine the hybridization conditions required by referring to textbooks such as the textbook mentioned above, or the following textbooks: Sambrook et al., “Molecular Cloning”, Cold Spring Harbor Laboratory, 1989; Hames and Higgins (Ed.) 1985, “Nucleic Acids Hybridization: A Practical Approach”, IRL Press at Oxford University Press, Oxford; Brown (Ed.) 1991, “Essential Molecular Biology: A Practical Approach”, IRL Press at Oxford University Press, Oxford. Alternatively, polynucleotide variants are obtainable by PCR-based techniques such as mixed oligonucleotide primer-based amplification of DNA, i.e. using degenerated primers against conserved domains of the polypeptides of the present invention. Conserved domains of the polypeptide of the present invention may be identified by a sequence comparison of the nucleic acid sequences of the polynucleotides or the amino acid sequences of the polypeptides of the present invention. Oligonucleotides suitable as PCR primers as well as suitable PCR conditions are described in the accompanying Examples. As a template, DNA or cDNA from bacteria, fungi, plants or animals may be used. Further, variants include polynucleotides comprising nucleic acid sequences which are at least 70%, at least 75%, at least 80%, at least 85%, at least 90%, at least 95%, at least 98% or at least 99% identical to the nucleic acid sequences shown in any one of SEQ ID NOs: 1, 3, 5, 7, 9, 11, 13, 15 or 17 whereby the variant nucleic acid sequence shall still encode a polypeptide be involved in cycloclavine synthesis and, in particular has a activity as specified above for the corresponding lead sequence (indicated in the SEQ ID Nos) from which the variant has been derived. Moreover, also encompassed are polynucleotides which comprise nucleic acid sequences encoding a polypeptide having an amino acid sequences which are at least 70%, at least 75%, at least 80%, at least 85%, at least 90%, at least 95%, at least 98% or at least 99% identical to the amino acid sequences shown in any one of SEQ ID NOs: 2, 4, 6, 8, 10, 12, 14, 16 or 18, whereby the variant nucleic acid sequence shall still encode a polypeptide be involved in cycloclavine synthesis and, in particular has a activity as specified above for the corresponding lead sequence (indicated in the SEQ ID Nos) from which the variant has been derived. The percent identity values are, preferably, calculated over the entire amino acid or nucleic acid sequence region. A series of programs based on a variety of algorithms is available to the skilled worker for comparing different sequences. In a preferred embodiment, the percent identity between two amino acid sequences is determined using the Needleman and Wunsch algorithm (Needleman 1970, J. Mol. Biol. (48):444-453) which has been incorporated into the needle program in the EMBOSS software package (EMBOSS: The European Molecular Biology Open Software Suite, Rice, P., Longden, I., and Bleasby, A, Trends in Genetics 16(6), 276-277, 2000), using either a BLOSUM 45 or PAM250 scoring matrix for distantly related proteins, or either a BLOSUM 62 or PAM160 scoring matrix for closer related proteins, and a gap opening penalty of 16, 14, 12, 10, 8, 6, or 4 and a gap extension penalty of 0.5, 1, 2, 3, 4, 5, or 6. Guides for local installation of the EMBOSS package as well as links to WEB-Services can be found at http://emboss.sourceforge.net. A preferred, non-limiting example of parameters to be used for aligning two amino acid sequences using the needle program are the default parameters, including the EBLOSUM62 scoring matrix, a gap opening penalty of 10 and a gap extension penalty of 0.5. In yet another preferred embodiment, the percent identity between two nucleotide sequences is determined using the needle program in the EMBOSS software package (EMBOSS: The European Molecular Biology Open Software Suite, Rice, P., Longden, I., and Bleasby, A, Trends in Genetics 16(6), 276-277, 2000), using the EDNAFULL scoring matrix and a gap opening penalty of 16, 14, 12, 10, 8, 6, or 4 and a gap extension penalty of 0.5, 1, 2, 3, 4, 5, or 6. A preferred, non-limiting example of parameters to be used in conjunction for aligning two nucleic acid sequences using the needle program are the default parameters, including the EDNAFULL scoring matrix, a gap opening penalty of 10 and a gap extension penalty of 0.5. The nucleic acid and protein sequences of the present invention can further be used as a “query sequence” to perform a search against public databases to, for example, identify other family members or related sequences. Such searches can be performed using the BLAST series of programs (version 2.2) of Altschul et al. (Altschul 1990, J. Mol. Biol. 215:403-10).

A polynucleotide comprising a fragment of any of the aforementioned nucleic acid sequences is also encompassed as a polynucleotide of the present invention. The fragments shall encode polypeptides which still are involved in cycloclavine synthesis and/or having an activity as specified above. Accordingly, the polypeptide may comprise or consist of the domains of the polypeptide of the present invention conferring the said biological activity. A fragment as meant herein, preferably, comprises at least 50, at least 100, at least 250 or at least 500 consecutive nucleotides of any one of the aforementioned nucleic acid sequences or encodes an amino acid sequence comprising at least 20, at least 30, at least 50, at least 80, at least 100 or at least 150 consecutive amino acids of any one of the aforementioned amino acid sequences. The variant polynucleotides or fragments referred to above, preferably, encode polypeptides retaining a significant extent, preferably, at least 10%, at least 20%, at least 30%, at least 40%, at least 50%, at least 60%, at least 70%, at least 80% or at least 90% of the activity exhibited by any of the polypeptide shown in any one of SEQ ID NOs: 2, 4, 6, 8, 10, 12, 14, 16 or 18. The activity may be tested as described in the accompanying Examples. Examples for the determination of enzymatic activities of enzymes in the clavine pathway from tryptophan to can be found in Gebler et al. Archives of Biochemistry and Biophysics, Volume 296, 1992, 308-313 (DAMT-synthase) Rigbers et a. J of Biological Chemistry Volume. 283, 26859-26868, 2008 (DMAT Methyl transferase), Wallwey et al. Arch Microbiol 192:127-134 2010. General enzyme assays for

The polynucleotides of the present invention either essentially consist of the aforementioned nucleic acid sequences or comprise the aforementioned nucleic acid sequences. Thus, they may contain further nucleic acid sequences as well. Preferably, the polynucleotide of the present invention may comprise in addition to an open reading frame further untranslated sequence at the 3′ and at the 5′ terminus of the coding gene region: at least 500, preferably 200, more preferably 100 nucleotides of the sequence upstream of the 5′ terminus of the coding region and at least 100, preferably 50, more preferably 20 nucleotides of the sequence downstream of the 3′ terminus of the coding gene region. Furthermore, the polynucleotides of the present invention may encode fusion proteins wherein one partner of the fusion protein is a polypeptide being encoded by a nucleic acid sequence recited above. Such fusion proteins may comprise polypeptides for monitoring expression (e.g., green, yellow, blue or red fluorescent proteins, alkaline phosphatase and the like) or so called “tags” which may serve as a detectable marker or as an auxiliary measure for purification purposes. Tags for the different purposes are well known in the art and comprise FLAG-tags, 6-histidine-tags, MYC-tags and the like.

However, the present invention, preferably, encompasses a polynucleotide which comprises one or more and, preferably, all of the nucleic acid sequences encoding the polypeptides having the aforementioned activities and are involved in the synthesis of cycloclavine. More preferably, the said polynucleotide, therefore, comprises:
(i) a nucleic acid sequence as shown in SEQ ID NO: 30 or 31,
(ii) a nucleic acid sequence being at least 70% identical to SEQ ID NO: 30 or 31 and encoding polypeptides being involved in cycloclavine synthesis, said polypeptides having an amino acid sequence which is at least 70% identical to the amino acid sequence as shown in SEQ ID NOs: 2, 4, 6, 8, 10, 12, 14, 16 or 18 and at least 80% identical to the amino acid sequence shown in SEQ ID NO: 6, or
(iii) a nucleic acid sequence which hybridizes under stringent conditions with the nucleic acid sequence of (i) or (ii).

The polynucleotide of the present invention shall be provided, preferably, either as an isolated polynucleotide (i.e. purified or at least isolated from its natural context such as its natural gene locus) or in genetically modified or exogenously (i.e. artificially) manipulated form. An isolated polynucleotide can, for example, comprise less than approximately 5 kb, 4 kb, 3 kb, 2 kb, 1 kb, 0.5 kb or 0.1 kb of nucleotide sequences which naturally flank the nucleic acid molecule in the genomic DNA of the cell from which the nucleic acid is derived. The polynucleotide, preferably, is provided in the form of double or single stranded molecule. It will be understood that the present invention by referring to any of the aforementioned polynucleotides of the invention also refers to complementary or reverse complementary strands of the specific sequences or variants thereof referred to before. The polynucleotide encompasses DNA, including cDNA and genomic DNA, or RNA polynucleotides.

However, the present invention also pertains to polynucleotide variants which are derived from the polynucleotides of the present invention and are capable of interfering with the transcription or translation of the polynucleotides of the present invention. Such variant polynucleotides include anti-sense nucleic acids, ribozymes, siRNA molecules, morpholino nucleic acids (phosphorodiamidate morpholino oligos), triple-helix forming oligonucleotides, inhibitory oligonucleotides, or micro RNA molecules all of which shall specifically recognize the polynucleotide of the invention due to the presence of complementary or substantially complementary sequences. These techniques are well known to the skilled artisan. Suitable variant polynucleotides of the aforementioned kind can be readily designed based on the structure of the polynucleotides of this invention.

Moreover, comprised are also chemically modified polynucleotides including naturally occurring modified polynucleotides such as glycosylated or methylated polynucleotides or artificial modified ones such as biotinylated polynucleotides.

In the studies underlying the present invention, advantageously, polynucleotides where identified which encode polypeptides involved in the synthesis of cycloclavine. In particular, a gene cluster has been identified in Botryotinia fuckeliana which comprises the nucleic acid sequences encoding all of the polynucleotides required for the synthesis of cycloclavine. The polynucleotides of the present invention are particularly suitable for the recombinant manufacture of cycloclavine or a precursor thereof. The total synthesis of its characteristic ring system, in which the ergoline skeletal carbon atoms 8, 9, and 10 form a cyclopropane ring in place of the 9,10 double bond, presents a considerable challenge. Thanks to the present invention, a complex compound such as cycloclavine or any of its precursors can be produced as a starting material for, e.g., further organic synthesis of alkaloids.

The present invention relates to a vector comprising the polynucleotide of the present invention.

The term “vector”, preferably, encompasses phage, plasmid, fosmid, viral vectors as well as artificial chromosomes, such as bacterial or yeast artificial chromosomes. Moreover, the term also relates to targeting constructs which allow for random or site-directed integration of the targeting construct into genomic DNA. Such target constructs, preferably, comprise DNA of sufficient length for either homolgous or heterologous recombination as described in detail below. The vector encompassing the polynucleotide of the present invention, preferably, further comprises selectable markers for propagation and/or selection in a host. The vector may be incorporated into a host cell by various techniques well known in the art. If introduced into a host cell, the vector may reside in the cytoplasm or may be incorporated into the genome. In the latter case, it is to be understood that the vector may further comprise nucleic acid sequences which allow for homologous recombination or heterologous insertion. Vectors can be introduced into prokaryotic or eukaryotic cells via conventional transformation or transfection techniques. The terms “transformation” and “transfection”, conjugation and transduction, as used in the present context, are intended to comprise a multiplicity of prior-art processes for introducing foreign nucleic acid (for example DNA) into a host cell, including calcium phosphate, rubidium chloride or calcium chloride co-precipitation, DEAE-dextran-mediated transfection, lipofection, natural competence, carbon-based clusters, chemically mediated transfer, electroporation or particle bombardment. Suitable methods for the transformation or transfection of host cells, including plant cells, can be found in Sambrook et al. (Molecular Cloning: A Laboratory Manual, 2^{nd} ed., Cold Spring Harbor Laboratory, Cold Spring Harbor Laboratory Press, Cold Spring Harbor, N.Y., 1989) and other laboratory manuals, such as Methods in Molecular Biology, 1995, Vol. 44, Agrobacterium protocols, Ed.: Gartland and Davey, Humana Press, Totowa, N.J. Alternatively, a plasmid vector may be introduced by heat shock or electroporation techniques. Should the vector be a virus, it may be packaged in vitro using an appropriate packaging cell line prior to application to host cells.

Preferably, the vector referred to herein is suitable as a cloning vector, i.e. replicable in microbial systems. Such vectors ensure efficient cloning in bacteria and, preferably, yeasts or fungi and make possible the stable transformation of plants.

Also preferably, the vector of the present invention is an expression vector. In such an expression vector, i.e. a vector which comprises the polynucleotide of the invention having the nucleic acid sequence operatively linked to an expression control sequence (also called “expression cassette”) allowing expression in prokaryotic or eukaryotic cells or isolated fractions thereof. An expression control sequence as referred to herein is a nucleic acid sequence which is capable of governing, i.e. initiating and controlling, transcription of a nucleic acid sequence of interest, in the present case the nucleic sequences recited above. Such a sequence usually comprises or consists of a promoter or a combination of a promoter and enhancer sequences. Expression of a polynucleotide comprises transcription of the nucleic acid molecule, preferably, into a translatable mRNA. Additional regulatory elements may include transcriptional as well as translational enhancers. The following promoters and expression control sequences may be, preferably, used in an expression vector according to the present invention. The cos, tac, trp, tet, trp-tet, lpp, lac, lpp-lac, lacIq, T7, T5, T3, gal, trc, ara, SP6, λ-PR or λ-PL promoters are, preferably, used in Gram-negative bacteria. For Gram-positive bacteria, promoters amy and SPO2 may be used. From yeast or fungal promoters ADC1, AOX1r, GAL1, MFα, AC, P-60, CYC1, GAPDH, TEF, rp28, ADH trpC, GAL10, cbh1, hfb2 amyB are, preferably, used further examples can be taken from MICROBIOLOGY AND MOLECULAR BIOLOGY REVIEWS 70, Pages: 583-ff 2006. For animal cell or organism expression, the promoters CMV-, SV40-, RSV-promoter (Rous sarcoma virus), CMV-enhancer, SV40-enhancer are preferably used. From plants the promoters CaMV/35S (Franck 1980, Cell 21: 285-294], PRP1 (Ward 1993, Plant. Mol. Biol. 22), SSU, OCS, lib4, usp, STLS1, B33, nos or the ubiquitin or phaseolin promoter. The expression control sequence shall be, preferably, operatively linked to the polynucleotide of the invention which means that the expression control sequence and the polynucleotide are linked so that the expression of the said polynucleotide can be governed by the said expression control sequence, i.e. the expression control sequence shall be functionally linked to the polynucleotide to be expressed. Accordingly, the expression control sequence and the polynucleotide to be expressed may be physically linked to each other, e.g., by inserting the expression control sequence at the 5′ end of the nucleic acid sequence to be expressed. Alternatively, the expression control sequence and the polynucleotide to be expressed may be merely in physical proximity so that the expression control sequence is capable of governing the expression of said polynucleotide. The expression control sequence and the polynucleotide to be expressed are, preferably, separated by not more than 500 bp, 300 bp, 100 bp, 80 bp, 60 bp, 40 bp, 20 bp, 10 bp or 5 bp. The polynucleotide in the context of an expression vector according to the present invention shall also, preferably, be operatively linked to a terminator sequence. A terminator as used herein refers to a nucleic acid sequence which is capable of terminating transcription. These sequences will cause dissociation of the transcription machinery from the nucleic acid sequence to be transcribed. Preferably, the terminator shall be active in the host cells referred to elsewhere herein. Suitable terminators are known in the art and, preferably, include polyadenylation signals such as the SV40-poly-A site or the tk-poly-A site. Other methods for the induction of genes or gene clusters involved in the production of natural compounds can be found in Brakhage et al.: Fungal Genet Biol 48, pp: 15-22 (2011).

Preferred expression vectors are known in the art such as Okayama-Berg cDNA expression vector pcDV1 (Pharmacia), pCDM8, pRc/CMV, pcDNA1, pcDNA3 (Invitrogene) or pSPORT1 (GIBCO BRL). Further examples of typical fusion expression vectors are pGEX (Pharmacia Biotech Inc; Smith 1988, Gene 67:31-40), pMAL (New England Biolabs, Beverly, Mass.) and pRIT5 (Pharmacia, Piscataway, N.J.), where glutathione S-transferase (GST), maltose E-binding protein and protein A, respectively, are fused with the recombinant target protein. Examples of suitable inducible nonfusion E. coli expression vectors are, inter alia, pTrc (Amann 1988, Gene 69:301-315) and pET 11d (Studier 1990, Methods in Enzymology 185, 60-89). The target gene expression of the pTrc vector is based on the transcription from a hybrid trp-lac fusion promoter by host RNA polymerase. The target gene expression from the pET 11d vector is based on the transcription of a T7-gn10-lac fusion promoter, which is mediated by a coexpressed viral RNA polymerase (T7 gn1). This viral polymerase is provided by the host strains BL21 (DE3) or HMS174 (DE3) from a resident λ-prophage which harbors a T7 gn1 gene under the transcriptional control of the lacUV 5 promoter. The skilled worker is familiar with other vectors which are suitable in prokaryotic organisms; these vectors are, for example, in E. coli, pLG338, pACYC184, the pBR series such as pBR322, the pUC series such as pUC18 or pUC19, the M113mp series, pKC30, pRep4, pHS1, pHS2, pPLc236, pMBL24, pLG200, pUR290, pIN-III113-B1, λgt11 or pBdCl, in Streptomyces pIJ101, pIJ364, pIJ702 or pIJ361, in Bacillus pUB110, pC194 or pBD214, in Corynebacterium pSA77 or pAJ667. Examples of vectors for expression in the yeast S. cerevisiae comprise pYep Sec1 (Baldari 1987, Embo J. 6:229-234), pMFa (Kurjan 1982, Cell 30:933-943), pJRY88 (Schultz 1987, Gene 54:113-123) and pYES2 (Invitrogen Corporation, San Diego, Calif.). Vectors and processes for the construction of vectors which are suitable for use in other fungi, such as the filamentous fungi, comprise those which are described in detail in: van den Hondel, C. A. M. J. J., & Punt, P. J. (1991) “Gene transfer systems and vector development for filamentous fungi, in: Applied Molecular Genetics of fungi, J. F. Peberdy et al., Ed., pp. 1-28, Cambridge University Press: Cambridge, or in: More Gene Manipulations in Fungi (J. W. Bennett & L. L. Lasure, Ed., pp. 396-428: Academic Press: San Diego). Further suitable yeast vectors are, for example, pAG-1, YEp6, YEp13 or pEMBLYe23. As an alternative, the polynucleotides of the present invention can be also expressed in insect cells using baculovirus expression vectors. Baculovirus vectors which are available for the expression of proteins in cultured insect cells (for example Sf9 cells) comprise the pAc series (Smith 1983, Mol. Cell Biol. 3:2156-2165) and the pVL series (Lucklow 1989, Virology 170:31-39).

Gene destruction in which homologous recombination is used can be carried out in the usual way, and the preparation of the vector which can be used for gene destruction and the introduction of the vector into a host are self-evident to those in the industry. Such vectors are also preferably envisaged as a vector according to the invention.

The present invention also relates to a host cell comprising the polynucleotide or the vector of the present invention.

Preferably, said host cell is a microorganism. More preferably, said microorganism is a bacterium, a yeast, an actinomycete, a a fungus, such as an ascomycete, a deuteromycete, or a basidiomycete, or a algae cell. Preferred bacteria to be used as host cells of the present invention are selected from the group consisting of: Escherichia coli and Bacilus subtilis. Preferred fungi are selected from the group consisting of: The genus Aspergillus such as Aspergillus japonicus, Aspergillus niger, Aspergillus oryzae, Aspergillus nidulans, Aspergillus fumigatus, Aspergillus aculeatus, Aspergillus caesiellus, Aspergillus candidus, Aspergillus carneus, Aspergillus clavatus, Aspergillus deflectus, Aspergillus fischerianus, Aspergillus flavus, Aspergillus glaucus, Aspergillus nidulans, Aspergillus ochraceus, Aspergillus parasiticus, Aspergillus penicilloides, Aspergillus restrictus, Aspergillus sojae, Aspergillus tamari, Aspergillus terreus, Aspergillus ustus, Aspergillus versicolor; the genus Penicillium such as Penicillium aurantiogriseum, Penicillium bilaiae, Penicillium camemberti, Penicillium candidum, Penicillium chrysogenum, Penicillium claviforme, Penicillium commune, Penicillium crustosum, Penicillium digitatum, Penicillium expansum, Penicillium funiculosum, Penicillium glabrum, Penicillium glaucum, Penicillium italicum, Penicillium lacussarmientei, Penicillium marneffei, Penicillium purpurogenum, Penicillium roqueforti, Penicillium stoloniferum, Penicillium uiaiense, Penicillium verrucosum, Penicillium viridicatum; the genus ergot or Claviceps such as Claviceps africana, Claviceps fusiformis, Claviceps paspali, Claviceps purpurea, Claviceps sorghi, Claviceps zizaniae. Preferred yeast are selected from the group consisting of: Schizosaccharomyces, Candida, and Pichia. Also preferably, said host cell can be a plant or animal host cell.

However, it will be understood that dependent on the host cell, further, enzymatic activities may be conferred to the host cells, e.g., by recombinant technologies. Accordingly, the present invention, preferably, envisages a host cell which in addition to the polynucleotide of the present invention comprises polynucleotides which are required or which facilitate the synthesis of tryptophan as the starting material for the synthesis of cycloclavine as referred to herein. More preferably, such an enzyme is the tryptophansynthase. More details on the biosynthesis of tryptophan may be found, e.g., in Radwanski 1995, Plant Cell 7(7): 921-934.

The present invention also relates to a method for the manufacture of a polypeptide encoded by a polynucleotide of the present invention comprising
a) cultivating the host cell of the present invention under conditions which allow for the production of the said polypeptide; and
b) obtaining the polypeptide from the host cell of step a).

Suitable conditions which allow for expression of the polynucleotide of the invention comprised by the host cell depend on the host cell as well as the expression control sequence used for governing expression of the said polynucleotide. These conditions and how to select them are very well known to those skilled in the art. The expressed polypeptide may be obtained, for example, by all conventional purification techniques including affinity chromatography, size exclusion chromatography, high pressure liquid chromatography (HPLC) and precipitation techniques including antibody precipitation. It is to be understood that the method may—although preferred—not necessarily yield an essentially pure preparation of the polypeptide. It is to be understood that depending on the host cell which is used for the aforementioned method, the polypeptides produced thereby may become posttranslationally modified or processed otherwise.

The present invention also relates to a polypeptide encoded by the polynucleotide of the present invention or a polypeptide which is obtainable by the aforementioned method of the present invention.

The term “polypeptide” as used herein encompasses essentially purified polypeptides or polypeptide preparations comprising other proteins in addition. Further, the term also relates to the fusion proteins or polypeptide fragments being at least partially encoded by the polynucleotide of the present invention referred to above. Moreover, it includes chemically modified polypeptides. Such modifications may be artificial modifications or naturally occurring modifications such as phosphorylation, glycosylation, myristylation and the like (Review in Mann 2003, Nat. Biotechnol. 21, 255-261). Currently, more than 300 posttranslational modifications are known (see full ABFRC Delta mass list at http://www.abrf.org/index.cfm/dm.home). The polypeptide of the present invention shall exhibit be involved in the synthesis of cycloclavine as specified above and, preferably, shall be capable of carrying out one of the enzymatic conversions referred to above and/or shall have an enzymatic activity referred to above.

The present invention relates to an antibody which specifically binds to the polypeptide of the present invention.

Antibodies against the polypeptides of the invention can be prepared by well known methods using a purified polypeptide according to the invention or a suitable fragment derived therefrom as an antigen. A fragment which is suitable as an antigen may be identified by antigenicity determining algorithms well known in the art. Such fragments may be obtained either from the polypeptide of the invention by proteolytic digestion or may be a synthetic peptide. Preferably, the antibody of the present invention is a monoclonal antibody, a polyclonal antibody, a single chain antibody, a chimerized antibody or a fragment of any of these antibodies, such as Fab, Fv or scFv fragments etc. Also comprised as antibodies by the present invention are bispecific antibodies, synthetic antibodies or chemically modified derivatives of any of the aforementioned antibodies. The antibody of the present invention shall specifically bind (i.e. does significantly not cross react with other polypeptides or peptides) to the polypeptide of the invention. Specific binding can be tested by various well known techniques. Antibodies or fragments thereof can be obtained by using methods which are described, e.g., in Harlow and Lane “Antibodies, A Laboratory Manual”, CSH Press, Cold Spring Harbor, 1988. Monoclonal antibodies can be prepared by the techniques originally described in Köhler 1975, Nature 256, 495, and Galfré 1981, Meth. Enzymol. 73, 3, which comprise the fusion of mouse myeloma cells to spleen cells derived from immunized mammals. The antibodies can be used, for example, for the immunoprecipitation, immunolocalization or purification (e.g., by affinity chromatography) of the polypeptides of the invention as well as for the monitoring of the presence of said variant polypeptides, for example, in recombinant organisms, and for the identification of proteins or compounds interacting with the proteins according to the invention.

The present invention also relates to a method for the manufacture of cycloclavine or a precursor thereof comprising:
a) cultivating the host cell of the invention under conditions which allow for the production of cycloclavine or a precursor thereof in said host cell; and
b) obtaining said cycloclavine or a precursor thereof from the said host cell,

The term “cycloclavine or a precursor thereof” has been defined already elsewhere in the specification in detail. Preferably, the precursor of cycloclavine is selected from the group consisting of: DMAT, Methyl-DMAT, Chanoclavine I, Chanoclavine aldehyde, and Agroclavine.

The term “cultivating” as used herein refers maintaining and growing the host cells under culture conditions which allow the cells to produce the said cycloclavine or a precursor thereof referred to above. This implies that the polynucleotide of the present invention is expressed in the host cell so that the polypeptide(s) encoded by the at least one nucleic acid sequence is present in the host cell in a biologically active form. Suitable culture conditions for cultivating the host cell are described in more detail in the accompanying Examples below. In particular, host cells of the present invention, preferably, can be cultured using, for example, glucose, sucrose, honey, dextrin, starch, glycerol, molasses, animal or vegetable oils and the like as the carbon source for the culture medium. Furthermore, soybean flour, wheat germ, corn steep liquor, cotton seed waste, meat extract, polypeptone, malt extract, yeast extract, ammonium sulfate, sodium nitrate, urea and the like can be used for the nitrogen source. The addition of inorganic salts which can produce sodium, potassium, calcium, magnesium, cobalt, chlorine, phosphoric acid (di-potassium hydrogen phosphate and the like), sulfuric acid (magnesium sulfate and the like) and other ions as required is also effective. Furthermore, various vitamins such as thiamine (thiamine hydrochloride and the like), amino acids such as glutamine (sodium glutamate and the like), asparagine (DL-asparagine and the like), trace nutrients such as nucleotides and the like, and selection drugs such as antibiotics and the like can also be added as required. Moreover, organic substances and inorganic substances can be added appropriately to assist the growth of the microorganism and promote the production of cycloclavine or the precursor thereof. The pH of the culture medium is, for example, of the order of pH 5.5 to pH 8. The culturing can be carried out with a method such as the solid culturing method under aerobic conditions, the concussion culturing method, the air-passing agitation culturing method or the deep aerobic culturing method, but the deep aerobic culturing method is the most suitable. The appropriate temperature for culturing is from 15° C. to 40° C., but in many cases growth occurs in the range from 22° C. to 30° C. The production of cycloclavine or its precursors differs according to the culture medium and culturing conditions, or the host which is being used, but with any culturing method the accumulation of cycloclavine reaches a maximum generally in from 2 to 10 days. The culturing is stopped when the amount of cycloclavine or its precursor in the culture reaches its highest level and the target material is isolated from the culture and refined for isolating cycloclavine or a precursor thereof from the culture material.

The term “obtaining” as used herein encompasses the provision of the cell culture including the host cells and the culture medium as well as the provision of purified or partially purified preparations thereof comprising the cycloclavine or a precursor thereof, preferably, in free form. More details on purification techniques can be found elsewhere herein below. The usual methods of extraction and refinement which are generally used in these circumstances, such as methods of isolation such as solvent extraction, methods involving ion exchange resins, adsorption or partition chromatography, gel filtration, dialysis, precipitation, crystallization and the like can be used either individually or in appropriate combinations. In particular, cycloclavine can be isolated from a cycloclavine containing medium or lysate using a known method for isolating cycloclavine. Preferably, the process for isolation disclosed by Furuta 1982, Agricultural and Biological Chemistry (1982), 46(7), 1921-2 is envisaged in accordance with the method of the present invention.

Finally, encompassed by the present invention is the use of the polynucleotide, the vector or the host cell of the invention, in general, for the manufacture of cycloclavine or a precursor thereof. As set forth previously, said precursor of cycloclavine is, preferably, selected from the group consisting of: DMAT, Methyl-DMAT, Chanoclavine I, Chanoclavine aldehyde, and Agroclavine.

All references cited in this specification are herewith incorporated by reference with respect to their entire disclosure content and the disclosure content specifically mentioned in this specification.

### FIGURES

FIG. 1 shows a schematic drawing of the cycloclavine gene cluster.

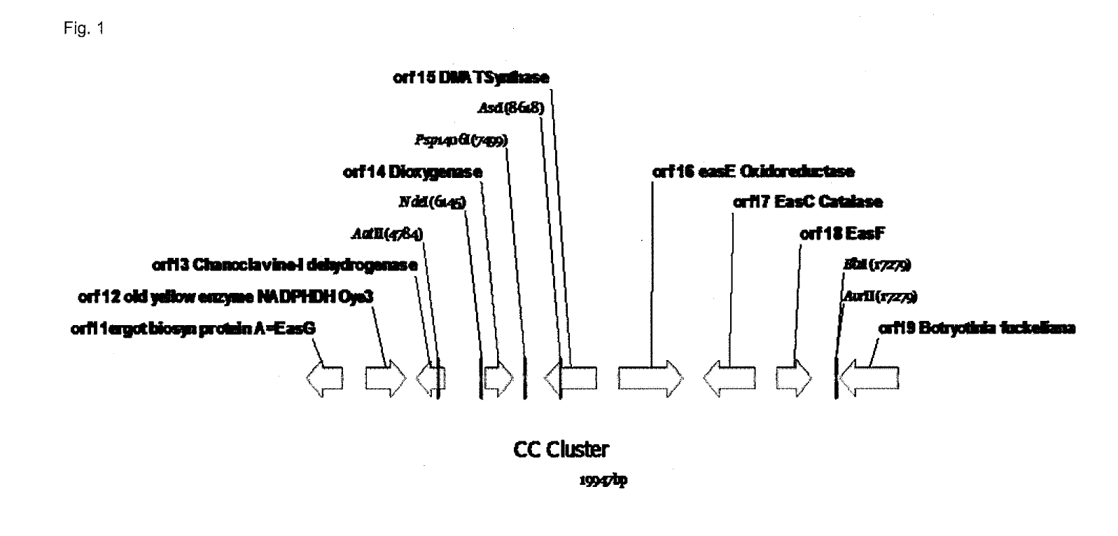

FIG. 2 shows a cycloclavine biosynthesis pathway.

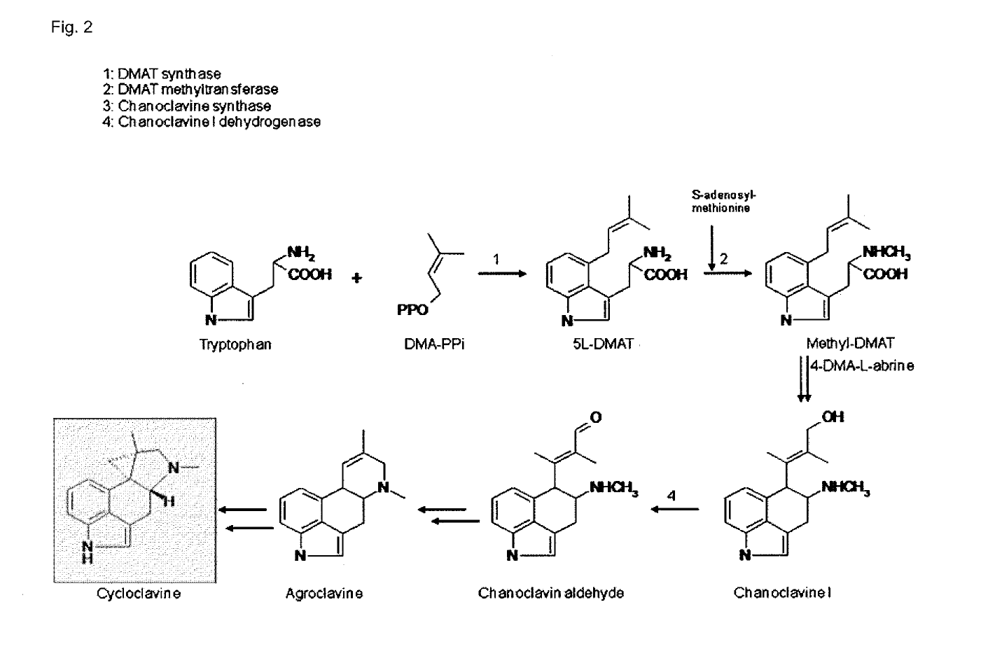

The invention will now be illustrated by the following Examples which, however, shall not be construed as limiting the scope of the invention.

### EXAMPLES

### Example 1

### Cloning of Expression Cassettes for the Recombinant Production of Cycloclavine

The first part of the cycloclavine gene cluster as described in Seq ID NO. 30 from base 58 to base 10057 is amplified using the primers:

(SEQ ID NO: 37)
P1_5′: CTAGTGCACCGCTTCCACCTATTCTACCTG
and
(SEQ ID NO: 38)
P2_3′: TCTCGGACCACCCTTCCCACAGCGGTGAAA

The second part part of the cycloclavine gene cluster as described in Seq ID NO. 30 from base 10072 to base 19947 is amplified using the primers:

(SEQ ID NO: 39)
P3_5′: AAGGGCAGGCCAAAAATCCGCTCCCAAGATGCT
and
(SEQ ID NO: 40)
P4_3′: TTGGGGTTCTGGTGGGTTGGGTTGGTGGGTTCT

PCR amplification is performed using Phusion polymerase (New England Biolabs Inc.) and using the parameters described in the manual. PCR is done using the following cycle parameters: 98° C. 1 min, (98° C. 20 sec, 66° C. for 30 sec 72° C. for 5 min) for 33 cycles and 5 min at 72° C.

PCR product of the anticipated size is excised from agarose gel (0.7%) and is cloned into the unique EcoRV restriction site of the vectors pHS Nat1 and into pHS delta Nat1 described in Seq ID No 28 and Seq ID No. 29, yielding the plasmids pHS Nat1 CC_{—}1 Seq ID NO. 35 and pHS delta Nat1 CC_{—}2 Seq ID NO.36. For the cotransformation of Cc cluster genes DNA fragments containing the part 1 CC_{—}1 (Seq ID NO₃₃) or part 2 CC_{—}2 (Seq ID NO34) of the CC cluster one of the parts is isolated by linearizing the plasmid pHS Nat1 CC_{—}1 (Seq ID NO33) using restriction enzyme digest from the Nat1 resistance gene containing vector pHS Nat1 CC_{—}1 (Seq ID No35) while the other part is isolated by SwaI restriction enzyme digest obtained from the pHS delta Nat1 CC_{—}2 (Seq ID No 36). Both fragments are mixed for the transformation that the fragment containing a Nat1 sequence is added at ⅕th or 1/10th of the amount of the part lacking the Nat1 sequence.

### Example 2

### Cloning of Overexpression Cassettes for the Recombinant Production of Cycloclavine Using Heterologous Promoters

Genes coding for the biosynthesis of cycloclavine are expressed using heterologous promoters such as the Ptrpc promoter from A. nidulans (Seq ID No.32). Promoter gene terminator fusions can be obtained by several technologies known to the person skilled in the art. Technologies are PCR fusion using overlapping primers for the promoter 3′ side and the gene 5′ side as well as promoter 5′ primers and gene-terminator 3′ primers. Methods for performing PCR fusion can be found in Nucl. Acids Res. (1989) 17: 4895. Another possible way to obtain promoter-gene terminator-fusion can be DNA synthesis 2009 by known methods (Czar et al. Trends in Biotechnology, 27, 63-72 and references therein).

Fragments containing promoter gene terminator fusions can be combined by several methods such as the Biobrick method, the Golden Gate Method, the SLIC Method, the CEPC Method (Li et al. Nature Methods 2007, 4: 251-256, Quan et al. PLOS ONE 4: e6441, Engler et al. PLOS ONE, 2008, 3 e3647, Engler et al. PLOS ONE, 2009 4 e5553).

All cassettes containing the promoter PtrpC (Seq ID NO. 32) and the genes coding for the orf 11 easG (Seq ID No. 1), the orf 12 easA oyeI (Seq ID No. 3), the orf 13 chanoclavine DH (Seq ID No. 5), the orf 14 (Seq ID No. 7), the orf 15 (Seq ID No. 9), the orf 16 (Seq ID No. 11), the orf 17 (Seq ID No. 13), the orf 18 (Seq ID No. 15), the orf 19 (Seq ID No. 17) are constructed by methods described above and cloned together or in two or more parts into the vector pHS1 nat1 or vector pHS1 delta nat1.

Fragments containing the all promoter-orf terminator cassettes can be isolated from the vectors using the SwaI digestion and are used for transformation of suitable fungal strains. An example for a gene fragment containing the promoter-orf-termination elements of orf 11, 12, 13, 14, 15, 16, 17, 18 and 19 is described in Seq ID No. 11

### Example 3

### Preparation of Expression Vector for Introduction into Hyphomycetes

Preparation of Expression Vector for Introduction into Hyphomycetes, pHS Nat1 Seq ID No. 28 and pHS delta Nat1 Seq ID No. 29 are produced by gene synthesis.

### Example 4

### Transformation of Cycloclavine Cluster DNA into Aspergillus japonicus and Penicillium chrysogenum

250 μl of a spore suspension are inoculated into a 500 ml flask with one baffle containing 100 ml of CCM medium (for one transformation experiment two flasks), and are incubated for 3 days at 27 C and 120 rpm. Mycelium is harvested by filtration and ished by adding 20 ml of PP-buffer (0.9 M NaCl). The dried mycelium is transferred in a sterile flask, 30 ml of 3% Glucanex solution is added. Incubation is performed for 2 h at 27 C and 100 rpm followed microscopic control of formed protoplasts. Filtration of protoplasts using a frit (pore size 1) is performed

Centrifugation of the protoplast suspension is performed for 5 min at 4 C and 2500 rpm. Supernatant is discarded and the protoplast pellet is dissolved in 10 ml PP-buffer. The protoplast suspension is centrifuged for 5 min at 4 C and 2500 rpm in a sterile tube The pellet is dissolved in 5 ml TP1-buffer (0.9 M NaCl, 50 mM CaCl2).

The protoplast titre is determined using a Abbe-Zeiss counting cell chamber and the titre is adjusted to 1×108 protoplasts/ml TP1-buffer. 10 microgram of linear DNA 1 and 2 are mixed with 50 μl of the protoplast suspension. 12.5 μl of TP2-buffer (25% PEG 6000, 50 mM CaCl2, 10 mM Tris, pH 7.5) are added and incubated for 20 min on ice. 500 μl of TP2-buffer are added, mixed gently and incubated 5 min at RT. 1 ml TP1-buffer are added. 2×780 μl of the transformation approach are mixed with 4 ml topagar I (CCM+20% sucrose+0.8% agar) and poured on CCMS plates. Plates are incubated at 27 C over night. All plates with expect of the regeneration controls are overlayed with 11 ml topagar II (0.8 M NaCl, 0.8% agar+Nourseothricine 50 μg/ml) and are incubated for >6 days at 27 C. Clones resistant against Nourseothricin are isolated, purified and analyzed for CC production in shake flask experiments.

### Example 5

### Transformation of Cycloclavine Cluster DNA into Aspergillus niger and Aspergillus oryzae

Aspergillus oryzae (strain) is cultured for 1 week at 30° C. in CD-Met (containing 40 μg/ml L-methionine) agar culture medium. Conidia (>10⁸) are recovered from the Petri dish and inoculated into 100 ml of YPD liquid culture medium in a 500 ml flask. After culturing for 20 hours (30° C., 180 rpm), an aegagropila-like biomass is obtained. The biomass is collected on a 3G-1 glass filter and washed with 0.8M NaCl and then de-watered thoroughly and suspended in TF solution I (protoplastizing solution) and shaken for 2 hours at 30° C., 60 rpm. The material is examined with a microscope every 30 minutes and the presence of protoplasts is confirmed. Subsequently, the culture liquid is filtered and the protoplasts are recovered by centrifugal separation (2000 rpm, 5 minutes) and then washed with TF solution II. After washing, 0.8 vol of TF solution II and 0.2 vol of TF solution III are added and admixed and a protoplast suspension is obtained.

Plasmid DNA (10 μg) is added to 200 μl of this liquid suspension and left to stand over ice for 30 minutes, TF solution III (1 mL) is added and then mixed gently. Subsequently the mixture is left to stand for 15 minutes at room temperature and the plasmid DNA is introduced into the aforementioned protoplasts. TF solution II (8 mL) is added and the mixture is centrifuged (5 minutes at 2,000 rpm) and 1 to 2 ml of residual protoplast is recovered. The recovered protoplast liquid is dripped into re-generating culture medium (lower layer), the regenerating culture medium (upper layer) is poured in and, after mixing by rotating the Petri dish, the mixture is cultured for from 4 to 5 days at 30° C. The clones which emerged are isolated in regenerating culture medium (lower layer) and the transfectants (Aspergillus oryzae (ATCC 1015 and Aspergillus oryzae ATCC 42149) are obtained by successive purification.

The abovementioned TF solution I (protoplastizing solution) is prepared using the composition indicated below.

| Compound | Concentration | |
|---|---|---|
| Yatalase (Produced by the | 25 | mg/ml |
| Takara-Bio Co.) | | |
| Ammonium sulfate | 0.65M | |
| Maleic Acid - NaOH | 55 | mM |

The abovementioned composition is prepared (pH 5.6) and then subjected to filtration sterilization. The abovementioned TF solution II is prepared using the composition indicated below.

| Compound | |
|---|---|
| 1.1M Sorbitol | |
| 50 mM CaCl₂ | 10 ml 1M CaCl₂ (1/20) |
| 35 mM NaCl | 1.4 ml 5M NaCl |
| 10 mM Tris-HCl | 2 ml 1M Tris-HCl (1/100) |
| Up to total volume | 200 ml |

The abovementioned composition is prepared and then subjected to autoclave sterilization. The abovementioned TF solution III is prepared using the composition indicated below.

| Compound | | |
|---|---|---|
| 60% PEG 4000 | 6 | g |
| 50 mM CaCl₂ | 500 μl 1M CaCl₂ (1/20) | |
| 50 mM Tris-HCl | 500 μl 1M Tris-HCl (1/100) | |
| Up to total volume | 10 | ml |

The abovementioned composition is prepared and then subjected to filtration sterilization.

The abovementioned culture medium is prepared using the composition indicated below.

| | |
|---|---|
| Compound | Concentration |

| Sorbitol (MW = 182.17) | 218.6 | g | 1.2M | |
|---|---|---|---|---|
| NaNO₃ | 3.0 | g | 0.3% | (w/v) |
| KCl | 2.0 | g | 0.2% | (w/v) |
| KH₂PO₄ | 1.0 | g | 0.1% | (w/v) |

| | | |
|---|---|---|
| MgSO₄•7H₂O | 2 ml of 1M MgSO₄ | 0.05% 2 mM |

| | | | | |
|---|---|---|---|---|
| Trace Elements Solution | 1 | ml | | |
| Glucose | 20.0 | g | 2% | (w/v) |
| Up to the total volume | 1 | L | | |

The abovementioned composition (pH 5.6) is prepared and then subjected to autoclave sterilization.

### Example 6

### Transformation of A. nidulans and A. fumigatus

Protoplasts are prepared from five cellophane cultures of A. nidulans (ATCC 11414, ATCC 10864) or A. fumigatus (ATCC 46645) as described in Ballance et al., Biochem. Biophys. Res. Commun. I 12 (1983) 284-2X9. After filtration through nylon filter cloth (Gallenkamp, GMX-500-V) and sintered glass (porosity I), the protoplasts are centrifuged at 1000×g for 5 min and then washed twice with 0.6 M KC1 and once with 0.6 M KCl, 50 mM CaCl. The protoplasts are resuspended in 0.2 ml of 0.6 M KCl, 50 mM CaCl (0.5-5×10⁸ ml and then 50-4 aliquots are dispensed into screw-capped tubes (Sarstedt). DNA (1 pg) is then added, followed by 12.5˜1 25% PEG 6000 (BDH), 50 mM CaCl, 10 mM Tris' HCl, pH 7.5. After 20 min incubation on ice, 0.5 ml of the above PEG solution is added and the mixture left at room temperature for 5 min. One ml of 0.6 M KCl, 50 mM CaCl, is added and aliquots are added to molten minimal medium containing KC1 (0.6 M) and agar (2% w/v) which is then poured over minimal agar plates. When necessary, the transformation mixture is diluted in 0.6 M KCl, 50 mM CaCl. The efficiency of regeneration is assessed by plating aliquots of a 10⁻³ dilution of the final transformation mixture in complete medium containing KCl and nourseothricin. All plates with expect of the regeneration controls are overlayed with 11 ml topagar 11 (0.8 M NaCl, 0.8% agar+Nourseothricine 50 μg/ml) and are incubated for >6 days at 27 C.

Clones capable of growing on the antibiotic are isolated, purified by repeated incubation on Nourseothricin containing agar plates and are used for Cyclolcavine production experiments.

### Example 7

### Growth of Fungal Strains after Transformation with DNA

Growth of Fungal Strains after Transformation with DNA Media and Cultivation of Microorganisms:

A. nidulans, A. fumigatus, A. japonicus SAITO IFO 4060, Penicillium chrysogenum (ATCC11500) and A. niger (ATCC 10864) strains that are successfully transformed with the genes of the cycloclavine cluster are cultivated at the appropriate incubation temperature (26° C. for Penicillium chrysogenum, 30° C. for A. niger and A. japonicus, 37° C. for A. fumigatus and A. nidulans) in YG (0.5% Yeast extract, 2% glucose), complete medium, or Aspergillus minimal medium with 1% (w/v) glucose as the carbon source and 5 mM of sodium glutamate as the nitrogen source and tryptophan (Biophys Acta 113:51?56).

### B. Alternatively the Strains are Grown on a Medium Containing

Glucose-monohydrate 80 g/l, defatted wheat germ meal 10 g/l, defatted soy bean meal 16 g/l, L-glutamate 3 g/l, NaCl 1.25 g/l, CaCO3 1.5 g/l, silicon oil KM-72 0.03 g/l. Alternatively the strains are grown in a medium containing 30 g/l Mannitol, 10 g/l glucose, 10 g/l succinic acid, 1 g/l KH2PO4 0.3 g/l MgSO4*7H2O, with NH4OH to adjust the pH to 5.6. Alternatively the strains are grown in a medium that promotes the synthesis of ergotamines (Hernandez, Process Biochemistry 1993 28 23-27) In all cases L-tryptophan and or mevalonic acid can be added in suitable amounts to increase the amount of produced compounds from the pathway including CC. Solid media contained 1.5% Bacto-agar or, in the case of minimal agar plates, Difco-agar. If required, p-aminobenzoic acid (0.11 mM), nourseothricine (50 μg/ml) are added).

Clones resistant against the antibiotic are grown in 250 ml baffled shake flask with a power stroke of 5 cm at 160-250 rpm. 25 ml medium is inoculated with freshly grown mycelium and incubated for 7 d at the appropriate incubation temperature (26° C. for Penicillium, 30° C. for A. niger, 37° C. for A. fumigatus and A. nidulans). All cycloclavine production experiments can be conducted in the presence of tryptophan addition as well as mevalonic acid addition for improved cycloclavine synthesis.

Cells as well as broth are harvested and are extracted as described in Furuta, Takaki; Koike, Masami; Abe, Matazo, Agricultural and Biological Chemistry (1982), 46, 1921-22

### Example 8

### CC Produced by the Transformed Fungal Strains can be Analyzed by a Suitable HPLC Method

Cc is analyzed by the following HPLC method: An injection volume of a sample size of 2 μl is injected into a ROD-HLPC column, 50×4.6 mm (Merck KGa Darmstadt Germany) at a temperature of 40° C. For the elution a solvent as follows is used: acetonitril+0.1% TFA; water+0.1% TFA. The flow rate is set to 1.8 ml/min, detection of eluting compounds is performed by electrochemical detection. A standard of CC obtained from A. japonicus SAITO IFO4060 is used for the calibration of the HPLC.

1. A polynucleotide comprising one or more nucleic acid sequences selected from the group consisting of:
a) a nucleic acid sequence having a nucleotide sequence as shown in SEQ ID NO: 1, 3, 5, 7, 9, 11, 13, 15 or 17;
b) a nucleic acid sequence encoding a polypeptide having an amino acid sequence as shown in SEQ ID NOs: 2, 4, 6, 8, 10, 12, 14, 16 or 18;
c) a nucleic acid sequence being at least 70% identical to the nucleic acid sequence as shown in SEQ ID NO: 1, 3, 7, 9, 11, 13, 15 or 17 or a nucleic acid sequence being at least 80% identical to the nucleic acid sequence shown in SEQ ID NO: 5, wherein said nucleic acid sequence encodes a polypeptide being involved in cycloclavine synthesis;
d) a nucleic acid sequence encoding a polypeptide being involved in cycloclavine synthesis and having an amino acid sequence which is at least 70% identical to the amino acid sequence as shown in SEQ ID NOs: 2, 4, 8, 10, 12, 14, 16 or 18 or at least 80% identical to the amino acid sequence shown in SEQ ID NO: 6; and
e) a nucleic acid sequence which is capable of hybridizing under stringent conditions to any one of a) to d), wherein said nucleic acid sequence encodes a polypeptide being involved in cycloclavine synthesis.

2. The polynucleotide of claim 1, wherein said polynucleotide comprises:
(i) a nucleic acid sequence as shown in SEQ ID NO: 30 or 31,
(ii) a nucleic acid sequence being at least 70% identical to SEQ ID NO: 30 or 31 and encoding polypeptides being involved in cycloclavine synthesis, said polypeptides having an amino acid sequence which is at least 70% identical to the amino acid sequence as shown in SEQ ID NOs: 2, 4, 8, 10, 12, 14, 16, or 18 and at least 80% identical to the amino acid sequence shown in SEQ ID NO: 6, or
(iii) a nucleic acid sequence which hybridizes under stringent conditions with the nucleic acid sequence of (i) or (ii).

3. A vector comprising the polynucleotide of claim 1.

4. The vector of claim 3, wherein said vector is an expression vector.

5. A host cell comprising the polynucleotide of claim 1 or the vector of claim 1.

6. The host cell of claim 5, wherein said host cell is a bacterial cell, a fungi cell, a yeast cell, a plant cell, or algae cell.

7. A method for the manufacture of a polypeptide encoded by a polynucleotide of claim 1 comprising
a) cultivating a host cell comprising the polynucleotide of claim 1 or comprising a vector comprising the polynucleotide of claim 1 under conditions which allow for the production of the said polypeptide; and
b) obtaining the polypeptide from the host cell of step a).

8. A polypeptide encoded by the polynucleotide of claim 1.

9. An antibody that specifically binds to the polypeptide of claim 8.

10. A method for the manufacture of cycloclavine or a precursor thereof comprising:
a) cultivating the host cell of claim 5 under conditions which allow for the production of cycloclavine in said host cell; and
b) obtaining said cycloclavine from the said host cell,

11. The method of claim 10, wherein said precursor of cycloclavine is selected from the group consisting of: DMAT, Methyl-DMAT, Chanoclavine I, Chanoclavine aldehyde, Agroclavine, and Festuclavine.

12. (canceled)

13. (canceled)

14. A polypeptide prepared by the method of claim 7.

15. An antibody that specifically binds to the polypeptide of claim 14.


SEQUENCE LISTING (40 sequences)

SEQ ID NO: 1 - DNA, 1125 bp, Aspergillus japonicus SAITO IFO 4060
  misc_feature (1)..(1125): orf11
atgaccatcctcgtcctcggcggccgcggcaagaccgccagccgcctctcagccctcctgcacgaagcccagatccccttcgtcgtgggcacctcctccacggccccgatcgccacctaccgaaccgcccacttcgactggctgaacaaagacacctggaccaacgcgtgggaccaggccccggagcccatcaccgctatctacctcgtaggcgcccacgccccggagttcgtgcacccgatgttcgagttcatcgacctggcgcacagccggggcgtgcggcggtacgcgctcgtcagcgcgagcaacaccgaaaagggggatccgatgatgggggaggcgcacgcgcacctggactcgaaagagggggtcaagtatgtggtgttgcggccgacgtggtttatgggtgcgttgtctggcttttcccttgccgtttcctcgcgtttccctctgagaaggggattggcgtgttttcctatctcgaatactttccccctgcccctaacccccaaatcccaaagtcaggactaattatggagtgaaacagaaaaccacatcgaagacccccacctcagctggatcaagacggagaacaagatctactcggccacgggcccgggcaagatccccttcgtctcggcggatgatatcgcccgcgtcgccttccacgccctgacgcagccgctcacccagaaggagcagcagtacatggttctgggcccggatctgctgtcctatgctgatgtttgtttcttcccaccttccaccccccacctcccatctttccctatctcctaccacggctatccgattcccagataagggtatgctcatgacgagaatgcgatgatactaatgctggagggaaaacagatggccgagatcctcaccgacctcctgggccggaagattacgcacgtcaccctgcccgccgagaagttcggccagttcctggtcgacacggccgggttgcccccggactttgcggagatgctggcgacgatggaggcggcggtggcgacgggggcggaggagcgggtcagccaagatgtcgagaaggtgaccgggcggccgccgcggcggtttgtggattacgcgagggagaatcgcgagcattggttgtga

SEQ ID NO: 2 - protein, 284 aa, Aspergillus japonicus SAITO IFO 4060
  CHAIN (1)..(284): easA orf11
MTILVLGGRGKTASRLSALLHEAQIPFVVGTSSTAPIATYRTAHFDWLNKDTWTNAWDQAPEPITAIYLVGAHAPEFVHPMFEFIDLAHSRGVRRYALVSASNTEKGDPMMGEAHAHLDSKEGVKYVVLRPTWFMENHIEDPHLSWIKTENKIYSATGPGKIPFVSADDIARVAFHALTQPLTQKEQQYMVLGPDLLSYADMAEILTDLLGRKITHVTLPAEKFGQFLVDTAGLPPDFAEMLATMEAAVATGAEERVSQDVEKVTGRPPRRFVDYARENREHWL

SEQ ID NO: 3 - DNA, 1239 bp, Aspergillus japonicus SAITO IFO 4060
  misc_feature (1)..(1239): orf12
atgacgatcgacccctcctcccccggggcgcccgcctcgaagctcttccaacccatgaagataggcctatgtcacctccagcaccgcctcatcatggcccccaccacccgctaccgcgccgacagcaccgcgacgcccctccccttcgtgcaagaatactacgcccaacgcgccgccgttccggggacactcctgatcaccgaagccaccgacatctccccgcaggcggccggagaaccgcacatcccaggcctgtacagcgcaacccagatcgccgcgtggcgggggatcgtctccgccgtgcaccgccggggctgctacatcttctgccagctgtgggcgaccggacgggcggcggaccccgccctgttgaaggagaaggggttccctctcgtctcgagcagcgatgtgcccatcgagactgaatcgcagcagccgcagccgctgacgcaggccgagatcgaggggtacatcgcggacttcgtgacggcggcaagggtagcggtccacgaggtggggttcgatggggtggagctgcacggcgccaacggatacctcatcgatcagttcacgcagtggccgtgcaaccagcggacggattgctggggcgggagcatcgagaaccgcgcgcggtttgcgctggcggtcactcgcgcggtggtggatgccgtcggggcggagcgcgtcggggttaagctgtcgccctggagtcagtacgccgggatgggggtgatggctgatttggtgccgcagtttgagttcttgatcaaaaggttacgggaggagtacaatgatggtcctgggctggcgtacttgcatctggcgaattcaaggtggttggatgaggagacttcccatccggatccgcatcatgaggtcttcgtgcgcgcgtggagggggtcgggcagggatggtattgaggcgaagaaagaggcgcctccggtgatcctggccggcggctatgatgctgtctcggcgcccgaggtcgctgataaggtgttcaaggaagaggacaatgtcgccgtcgcgtttgggcgatattttatctcgacgcccgacctgccgttccgcatgcaacggggcattccattgcagaagtatgatcgcgcctcgttctatacctcgcttttgaaagaaggttatctggattatccgtacagtgcggagtatgttgcggagtttgggcagaggctgaagaagaaggaggcggcggcgacagggtcgatatgtcagggttga

SEQ ID NO: 4 - protein, 412 aa, Aspergillus japonicus SAITO IFO 4060
  CHAIN (1)..(412): orf12
MTIDPSSPGAPASKLFQPMKIGLCHLQHRLIMAPTTRYRADSTATPLPFVQEYYAQRAAVPGTLLITEATDISPQAAGEPHIPGLYSATQIAAWRGIVSAVHRRGCYIFCQLWATGRAADPALLKEKGFPLVSSSDVPIETESQQPQPLTQAEIEGYIADFVTAARVAVHEVGFDGVELHGANGYLIDQFTQWPCNQRTDCWGGSIENRARFALAVTRAVVDAVGAERVGVKLSPWSQYAGMGVMADLVPQFEFLIKRLREEYNDGPGLAYLHLANSRWLDEETSHPDPHHEVFVRAWRGSGRDGIEAKKEAPPVILAGGYDAVSAPEVADKVFKEEDNVAVAFGRYFISTPDLPFRMQRGIPLQKYDRASFYTSLLKEGYLDYPYSAEYVAEFGQRLKKKEAAATGSICQG

SEQ ID NO: 5 - DNA, 886 bp, Aspergillus japonicus SAITO IFO 4060
  misc_feature (1)..(886): orf13
atgaccgtcgtcaactcgcgcatctttgcagtcaccggcggcgcctccggcattggcgcggccacctgccgcctcctcgccgcgcgcggcgcagcggtcatctgcatcggggacatctcgcccaagggcttcgagagcgtcaccagcgacatccaggccctcaacccggccaccaaggtgcactgcacccggctggacgtctcctcgtccgccgaggttgacgcctggctacagaccatcgtgcagaccttcggggacttgcacgggctggccaacgtcgcgggcatcgcccagccggcgggcgcccgccaggccccggccctgctcgaggagacggacgaggaatggcagcgcgtgttcaaggtcaacctggacggggtcttctactgcacgcgcgcaggtatccgcgcgatgaaggcccttccggggcccaaggaccgcgccatcgtcaacatcggtagcatcgccgcgtttattcacttgccggatgcctacgcctacgggacctccaagggcgcctgcgcgtacttcaccagctgtgcggccatggatgcctggcccctggggattcggatcaacagtgtctcgccgggtaagtgtctttcgtgctgtttattgtatagatctatggacagaggagaggaggatgaccctttggcgaggggggaaagctaacattgaggaaacaggtatcaccgacaccccggctctgccgcagttcatgccccaggcgaagtccatcgatgaggtcaaggaagcgtacatcaaggaggggttccctatcattcagcctgaggatgtggccaggacgattgtctggctgctcagcgaggattcccgcccggtgtatggggccaatatcaatgttggggctgctactccttga

SEQ ID NO: 6 - protein, 262 aa, Aspergillus japonicus SAITO IFO 4060
  CHAIN (1)..(262): orf13
MTVVNSRIFAVTGGASGIGAATCRLLAARGAAVICIGDISPKGFESVTSDIQALNPATKVHCTRLDVSSSAEVDAWLQTIVQTFGDLHGLANVAGIAQPAGARQAPALLEETDEEWQRVFKVNLDGVFYCTRAGIRAMKALPGPKDRAIVNIGSIAAFIHLPDAYAYGTSKGACAYFTSCAAMDAWPLGIRINSVSPGITDTPALPQFMPQAKSIDEVKEAYIKEGFPIIQPEDVARTIVWLLSEDSRPVYGANINVGAATP

SEQ ID NO: 7 - DNA, 882 bp, Aspergillus japonicus SAITO IFO 4060
  misc_feature (1)..(882): orf14
atgaccatcaccgagacctccaagccgacgctgcgccgcatcccccggagcgccggcgacgaggccatcttccaagtcctgcaggaagacggagtcgtcgtgatcgaaggattcatgagcgcggaccaggtccgccgcttcaacggggagatcgacccgcacatgaagcagtgggaactgggccagaagtcgtaccaggagagctacctggcggggatgcgacagctgagcagcctgccgctgttcagcaagctcttccgggacgagctgatgaacgacgagctcctgcacgggctgtgcaagcggctgttcgggcccgagtccggcgactactggctgacgacctcctcggtgctggagacagagccgggctaccacgggcaggagctgcaccgcgagcacgacgggatccccatctgcaccaccctgggccgccagagccccgagtccatgctcaacttcctcaccgcgctgaccgacttcaccgcggagaacggcgccacccgcgtcctgccgggcagtcacctctgggaggacttcagcgccccgccccccaaggccgacaccgccatccccgccgtcatgaaccccggcgatgccgttctcttcaccggcaagaccctgcacggcgccggcaagaacaacaccaccgacttcctgcgccgcggcttccccctgatcatgcagtcgtgccagttcacccctgtcgaggcctcggtggccctgccccgtgagctggtcgagaccatgacccccctggcccagaagatggtcggctggcggaccgtctccgctaagggggtggatatctggacgtatgatctgaaggatctggcgacaggaattgatctcaagtcgaaccaggtggcgaaaaaggcgtga

SEQ ID NO: 8 - protein, 293 aa, Aspergillus japonicus SAITO IFO 4060
  CHAIN (1)..(293): orf14
MTITETSKPTLRRIPRSAGDEAIFQVLQEDGVVVIEGFMSADQVRRFNGEIDPHMKQWELGQKSYQESYLAGMRQLSSLPLFSKLFRDELMNDELLHGLCKRLFGPESGDYWLTTSSVLETEPGYHGQELHREHDGIPICTTLGRQSPESMLNFLTALTDFTAENGATRVLPGSHLWEDFSAPPPKADTAIPAVMNPGDAVLFTGKTLHGAGKNNTTDFLRRGFPLIMQSCQFTPVEASVALPRELVETMTPLAQKMVGWRTVSAKGVDIWTYDLKDLATGIDLKSNQVAKKA

SEQ ID NO: 9 - DNA, 1604 bp, Aspergillus japonicus SAITO IFO 4060
  misc_feature (1)..(1604): orf15
atgactgctgggcaaggcatcaagacgggcaatgcctctgactgtgaagtttaccgcacgctgagcgtcgccctcgactttgccaaccaggatgaggagctctggtggcacagcaccgcgcccatgtttgctcagatgctgcagtccaccaactacaacctccatgctcagtacaagcacctgctcatctacaagaaaaacgtcatccctttcctcggggtttatcccacaaatgacaagccccggtggctaagcatcctgacccgatacggcaccccctttgagctgagcctgaactgctccggcccactggtgcgctacacctacgagcccatcaacgcggccacggggacggcgcgcgacccgttcaacacccacgctgtctgggacagtctggaacagctgatggcgctgcagagcgggattgacctggacctcttccgccacttcaagaacgacctcaccctcagcgctgaggaatctgagtacctgtacaagaacaacctggtgggcgagcagatccggactcagaacaaactggcgctggatctccaggacggcgagttcgtggtcaagacctacatctacccggccctgaagtcgctcgccacgggcaggtccatccacgagctggtcttcgggtctgccttccgctggtccaagcagtacccggagctccgcaagcccctggacacactggagcagtacgtctactcgcggggccccagcagcacggccagcccgcgtctcctctcctgcgacctgatcgaccccaccaagtcccgcatcaagatctacctgctggagcggatggtcaccctggaggccctggaggacctctggaccatggggggcgagcgcaccgacgcctcgaccctggcggggctcgagatgatccgcgagctgtgggagctgatccgcctgcccgcgggcttgcagtcgtatccggccccgtatctgcccatcgggaccatcccggacgagcagctgcccctgatggccaactacaccatccaccacgacgaccccgttccggagccgcaggtctacttcaccaccttcggccgcaacgacatgcagatcgccgacgcgctggccaccttcttcgagcgccgcggctggcacgagatggcgcgccagtacaaggccgagctgtgcagccactacccgcacgccgaccacgagaccctgaactacctgcacgcctacatctccttctcctaccgcaagaacaaaccctacctcagcgtgtatctgcagtcgctggagacgggcgactgggtcacttgtaagctctccccttccgtgttcctttcctgcacacttccccaccgtggatacatccctgatgatctgacctgttcgcgcagcatccttcaactctgtccatgtggaccccggcctctcagccaccgtccaagagctgtccaagctgaccaagaccgccgggaccaccgttcgcgagacgaaactcccactcaccccggatggatcggagcccggcgtcatcacccagtactaggaaatcatctcgttggaccggatttgccagcttcccgccggggagttgcaagtgctttgtatgtaggtaggggaactagatg

SEQ ID NO: 10 - protein, 534 aa, Aspergillus japonicus SAITO IFO 4060
  CHAIN (1)..(534): orf15
MTAGQGIKTGNASDCEVYRTLSVALDFANQDEELWWHSTAPMFAQMLQSTNYNLHAQYKHLLIYKKNVIPFLGVYPTNDKPRWLSILTRYGTPFELSLNCSGPLVRYTYEPINAATGTARDPFNTHAVWDSLEQLMALQSGIDLDLFRHFKNDLTLSAEESEYLYKNNLVGEQIRTQNKLALDLQDGEFVVKTYIYPALKSLATGRSIHELVFGSAFRWSKQYPELRKPLDTLEQYVYSRGPSSTASPRLLSCDLIDPTKSRIKIYLLERMVTLEALEDLWTMGGERTDASTLAGLEMIRELWELIRLPAGLQSYPAPYLPIGTIPDEQLPLMANYTIHHDDPVPEPQVYFTTFGRNDMQIADALATFFERRGWHEMARQYKAELCSHYPHADHETLNYLHAYISFSYRKNKPYLSVYLQSLETGDWVTCKLSPSVFLSCTLPHRGYIPDDLTCSRSILQLCPCGPRPLSHRPRAVQADQDRRDHRSRDETPTHPGWIGARRHHPVLGNHLVGPDLPASRRGVASALYVGRGTR

SEQ ID NO: 11 - DNA, 1993 bp, Aspergillus japonicus SAITO IFO 4060
  misc_feature (1)..(1993): orf16
atgggtcagtcacgagggatcttgggaggcgtgaggcaacttatcctggttattctggttggggcatacctcagccggctttcagcggtggatgacgaccgccacgactgtcgatgtcggccgggggagccatgctggcccacggtcagccactggagtactctgaacgagtccatcggcggtgctctggctcatgtcaaacccatcgctcacgtctgtcaccagtctggtcaggactcgtcggcttgcgagcaggtcttgcaagagagtcttgacagcaagtggcgggcgtcgcacacgggttagctcattccacctccctgaattcatctgacacacgggctcatccatcccacacaggcgcattgcaagactgggtctgggaaggtggcgcggaatccaaccagacatgttactacctgcggtccggtccggctggatcatgccaccagggccggattcccctctactccgcggctgtcaagtcggccagcgacgtgcagaaggtcgtggacttcactcgtcaacacaatttacgattagtgatccgcaacaccggccacgatgggagcggccgatccagtggaccggactctgtcgagatccacacccaccatctgaacagcgtgcaatatcaccccaacttccgccccgctggatcttctgagcgccagtcggctccaggacagccagcggtgacagtaggcgccggaatcctgcttggggacctctacgcccgcggcgcctcggagggatggatcgtagtgggcggggagtgtcccaccgtcggggcggccggcggattcctccagggcggaggagtatcgagttggctgagctacgcgcatggattggcagtggataatgtgcttgagtatgaagttgtgacggccaaggtgcgtgcgggttcatagcttccctccctccgcgctctaaccctaactaaccctccgcaaatgtccagggagagattgtcatcgccaacgcccatcaaaaccccgatctcttctgggcactgcgcgggggaggaggaggcacctttggcgtcgtgacccaggccacgcttcaagtccaccccgacctccccgtcagcgtcgccgacgccgtggtgacgggctcccgcgccgacgcgaccttctggtcccacggcgtcgccgccctcctgcgagcgctacaattcctcaacaaccacggcacggcaggccagttcatcctccgcaacaccgacgacgacacggtgcaagccagtctgacgatgtacttctcgaacctgaccgtccccgccgtcgccgacgagcgcatggacccgctccgccgcgccctcgaacacaacggacacccctaccagctcacctcgcggttcctgccccagatcagcagcaccttccggcacaccgcggatcgctacccggaggactacggaatcctgatgggctcggtcctggtgtcgctggacctgttcaattccgcgacgggccccgcggcgctggcgcagcacttcgcgcggctcccgatgacccccgacgatctcctgttcaccagcaacctgggcgggcgagtgtcctccctcaaccgcgacccggccagcacagccatgcatcccggctggcgtgacgccgcgcagttgctcaacttcgtccgcggggtcggggccccttctctcgcggctaaggccaccgccctccacgagctgcagacggtccagatggcgaggctgtatgagattgaaccggccttccagatctcgtaccgcaacctgggagatccctcggagcgccgcagtcgggaggtctattgggggtcgaactacgcgcggttagtggaggtcaagcggaggtgggatccggagggactgttcttcagcaagctggggattgatggtgatgcatgggacgcggaaggaatgtgtcgtcagacgcgccaaggggcgtggaatgtggcggtcaaatgggtccagagcttccttgggtctttgtctgcttctgtggtgtag

SEQ ID NO: 12 - protein, 621 aa, Aspergillus japonicus SAITO IFO 4060
  CHAIN (1)..(621): orf16
MGQSRGILGGVRQLILVILVGAYLSRLSAVDDDRHDCRCRPGEPCWPTVSHWSTLNESIGGALAHVKPIAHVCHQSGQDSSACEQVLQESLDSKWRASHTGALQDWVWEGGAESNQTCYYLRSGPAGSCHQGRIPLYSAAVKSASDVQKVVDFTRQHNLRLVIRNTGHDGSGRSSGPDSVEIHTHHLNSVQYHPNFRPAGSSERQSAPGQPAVTVGAGILLGDLYARGASEGWIVVGGECPTVGAAGGFLQGGGVSSWLSYAHGLAVDNVLEYEVVTAKGEIVIANAHQNPDLFWALRGGGGGTFGVVTQATLQVHPDLPVSVADAVVTGSRADATFWSHGVAALLRALQFLNNHGTAGQFILRNTDDDTVQASLTMYFSNLTVPAVADERMDPLRRALEHNGHPYQLTSRFLPQISSTFRHTADRYPEDYGILMGSVLVSLDLFNSATGPAALAQHFARLPMTPDDLLFTSNLGGRVSSLNRDPASTAMHPGWRDAAQLLNFVRGVGAPSLAAKATALHELQTVQMARLYEIEPAFQISYRNLGDPSERRSREVYWGSNYARLVEVKRRWDPEGLFFSKLGIDGDAWDAEGMCRQTRQGAWNVAVKWVQSFLGSLSASVV

SEQ ID NO: 13 - DNA, 1611 bp, Aspergillus japonicus SAITO IFO 4060
  misc_feature (1)..(1611): orf17
atggcgcccgatagcaagccgacctacaccacggcaaatggctgtcccttccacaagcctgagggtcctccggcggatggaaggacgctggcactcagcgaccaccatctggtcgataccctcgcccacttcaaccgggagaagatccccgagcgagtggtgcatgctaaaggagcgggagcctatggcgaatttgaggtatgaggcagcatatcctggtacagacgagggaaggaaccacgcccaagctgagttgctgacccggaccgtccattccaggtgacggccgacatctcggatatctgcgatatcgacatgctggtgggagtgggcaagaagaccccctgtgtgacgcggttctcgacgactggcctcgaacgcgggtccaacgagggaatgcgcgacctgaaaggcatggcctgcaagttctacaccaccgagggcaactgggactgggtgatgctcaacttccccttcttcttcatccgcgaccccgtcaagttccccagcctgatgcacgcccagcgccgggatccgcagaccaacctgctgaaccccaacatgtactgggactgggtgaccaacaaccacgagtcgctgcacatggtcctgctgcagttcagcgacttcgggaccatgttcaactggcggtccatgagcggctacatggcccacgcgtacaaatgggtgaagcccgacgggtcgttcaaatacgtccacatcttcctctcctccgatcgcgggcccaacttcaccgacggccagcaggccaagggcacgaacgacctggatccggaccacgccacccgcgacctgtacgaggccatcgagcgcggcgagtatccgacgtggacggcgagcgtgcaggtggtcgaccccaaggacgcgccgaacctggggtacaacatcctggacgtgaccaagcactggaacctgggcacgtaccccaaggacgtgacgctgatcccgcccaaggtcttcggcaagctgacgctcaagcgcaacccggccaactacttcgccgagatcgagcagctggcctactccccctcgaacatggtgcccggggtggccccctcggaggacccgatcctccaggcgcgcatcttcgcctacccggacgcccagcggtaccggctgggcgccaaccaccagcagatccccgtcaaccggtccgcccacaccttcaaccccatcgcgcgcgacggccagggcaccttcgacgccaactacggcgcccacccgggctttctgacccagcagcagcccgtccgcttcgccgagccccgcgagcccgaccccaagtacaacgagtggctcaaggagatccagtccaagtcctggctccagaccaccgagcacgactacaagttcgcccgcgacttctacgaggtcctgccggacttccgcgggcaggagttccaggacaccatggtccagaatatggtcgagtccgtcgcgcagacgcgcgcggagatccaaaagcaggtctacgagacctggaagctggtgagtccagcgctggcggcgcgaatccagaaaggggtggagacgctgctgcagaagagtgaggcgccggcggaggagtctttcatcccggcaagattgtag

SEQ ID NO: 14 - protein, 509 aa, Aspergillus japonicus SAITO IFO 4060
  CHAIN (1)..(509): orf17
MAPDSKPTYTTANGCPFHKPEGPPADGRTLALSDHHLVDTLAHFNREKIPERVVHAKGAGAYGEFEVTADISDICDIDMLVGVGKKTPCVTRFSTTGLERGSNEGMRDLKGMACKFYTTEGNWDWVMLNFPFFFIRDPVKFPSLMHAQRRDPQTNLLNPNMYWDWVTNNHESLHMVLLQFSDFGTMFNWRSMSGYMAHAYKWVKPDGSFKYVHIFLSSDRGPNFTDGQQAKGTNDLDPDHATRDLYEAIERGEYPTWTASVQVVDPKDAPNLGYNILDVTKHWNLGTYPKDVTLIPPKVFGKLTLKRNPANYFAEIEQLAYSPSNMVPGVAPSEDPILQARIFAYPDAQRYRLGANHQQIPVNRSAHTFNPIARDGQGTFDANYGAHPGFLTQQQPVRFAEPREPDPKYNEWLKEIQSKSWLQTTEHDYKFARDFYEVLPDFRGQEFQDTMVQNMVESVAQTRAEIQKQVYETWKLVSPALAARIQKGVETLLQKSEAPAEESFIPARL

SEQ ID NO: 15 - DNA, 1109 bp, Aspergillus japonicus SAITO IFO 4060
  misc_feature (1)..(1109): orf18
atgggcagcatcaatcccccgattttggacatccgccagtcgaccttcgagacctccatcccccaacaagtaaccgagggactcaccaaagaccccaaaaccctgccggcccttcttttctacagcggcgatggcatccagcactggacgcgccactcgtgcgccccggatttctacccccgccgcgaggagatcaacatcctccacagcgaagcggctcacatggcggcctcgataagccaggacagtgttgtggtggatctgggcagcgcgtatgcaagcacacacaatctacaatcaaagcccagcagaggtgtcggggaaatgaataactaaaccgcaaacccaatgcaccttatagatccctcgataaagtcctccccttcctgcacgccctcgaggcccaacagaagcgcgttcactactacgccctcgacctgagctacccggtgctcgcctcgacgctgtccgagatccccgtggcgcagttccagtatgtccagatcgccgcgctgcacggcaccttcgaggacgggctgcagtggctgcacgactcgcccgaggtccgcgagcgcccccaccacctgctgctgttcggcctcacgatcggcaacttctcacggcccaacgccgccgcctttttacggaatatcgccgaccgggcgctggcggcgtcccccgcggagagctccatcctcctcaccctggacagctgcaagatgccgaccaaggtgctgcgcgcgtacacggcggaaggggtggtgcctttcgcgctgacctcgttgagctatgccaatcagctcttctggccgggtggggagggaagggtgtttgatgaagaggaatggtatttccacagtgagtggaacttcgcgctggggcgccacgaggcggcgctcatcccccgggatcgcgatatcaccctcgggccgccgttggagcaggtcgtcgtgcgccggggtgagaaggtgaggtttggctgcagctacaagtatagtgctgcggagcgggagcatttgttcgctggggcggggttggagaatgtggcttcctggtcggccaagggctgtgatgttgctttttatcagttgaagatgcggcccgagtga

SEQ ID NO: 16 - protein, 339 aa, Aspergillus japonicus SAITO IFO 4060
  CHAIN (1)..(339): orf18
MGSINPPILDIRQSTFETSIPQQVTEGLTKDPKTLPALLFYSGDGIQHWTRHSCAPDFYPRREEINILHSEAAHMAASISQDSVVVDLGSASLDKVLPFLHALEAQQKRVHYYALDLSYPVLASTLSEIPVAQFQYVQIAALHGTFEDGLQWLHDSPEVRERPHHLLLFGLTIGNFSRPNAAAFLRNIADRALAASPAESSILLTLDSCKMPTKVLRAYTAEGVVPFALTSLSYANQLFWPGGEGRVFDEEEWYFHSEWNFALGRHEAALIPRDRDITLGPPLEQVVVRRGEKVRFGCSYKYSAAEREHLFAGAGLENVASWSAKGCDVAFYQLKMRPE

SEQ ID NO: 17 - DNA, 1837 bp, Aspergillus japonicus SAITO IFO 4060
  misc_feature (1)..(1837): orf19
atgcaccgccccctcacccccacccccaagccccaacccgcgcgcaagcgcctcctcatctgctgcgacggcacctggcaatccgccgtcgacggccaagagcgcatcccctcgaacgtgacgcggctctgccgcgccatcgacagcgtggggatcgacccgcacgaccaccagccctggcagcagatcgtctggtacgactccggggtgggcaccaacaccacggcggcgggcccgctgcgcaacttcaccgagggcgccctgggccaagggctggagggcaacatcgtcgaggcctaccacttctgcgccctgaactggaacccgggcgaccagatcctgtgctttgggttctcgcgcggcgcctacaccgcgcgcgccatcgccggcctcatctcggacatcggcatctgcccgccgcaccagatcgcgaatttcccggcgctgtggaaggtgtataagaagacgccgcccggggagcggttctatggcagtgatgcgtactatgagtttttccgggggcgccgatcctccttcggcggagaagagggggaagaggaggaggatggggcagaagaagaggagggtttttgggagtcggccgcagaatgggcatcgacccccgagtcacggcaggtgcaagtggtcggggtcttcgataccgtcggcaatctgggcttccccgagctgttcggccatgagctccctgcctggctgacctggactgataagcctgaatggcacaatgtcggtctttcgcctagtaagcaatccctccaggctatcattggctcaggatgagacgatactgatcccaaacccagacatcaagaatgcattccaggcgctggcgttggacgagcaccgccgactgttccgcccggcgatgttcttcgtcccggcgccgaagaatatcacggagagccaatcgcgagcgatcaagcaggaggcacggaacgccacccgcgagtggctaaaactcgtaagaagcggtgccgccagcaccgaggagttacacgcagccgagaaacgacgcaacgcggctgcccgtgagcttttggaatgggaggacagccagaaggagccgtcccagctgacgcaggtctggtttccggggttccacattcacatcgggggcggatccaaccagaccctcaagaacaagggcaacatggaagagatgtcccacatcgtcttcgcgtggatgctggaccagatccaggggtacgtctccctcgaccgcacgctcctccagctcgaggaaaagactcgagaggatcgtctgcggcggatcaacgaggcgctggaggcgtaccagaagaaagaacgtcgccggcagtccgagtcctgggcccagtggctgtggcacacagggcagggtctgattaccgccgtgcaacacccgctcaccccgtcgggcgatgctcccccggcctatcgccagatccgtcagtacggctgggccaccggcgacttgcccgacagcttcgacttcacctaccgcctgaacgggtcgctggcgcgcaggccgcgtcgctattttcgggatgtccgcacgaatgaggttctcgggcatacttgcgagtgcatccatcccacggtggggtttcgactgaagcacgtgccgggatatcgtccggctgggctgcgtgatggccagtatgctcgtcagcttagggctgacggccagggctatgagtattgcttcaggtaccccggtgagaaggatgaagaggtcttgccggagtgggagatggctagagatgagctctcctgggaacggtgggttgtcaagggaaaggaggcgtcggcgtatgtggatagcttattctattga

SEQ ID NO: 18 - protein, 591 aa, Aspergillus japonicus SAITO IFO 4060
  CHAIN (1)..(591): orf19
MHRPLTPTPKPQPARKRLLICCDGTWQSAVDGQERIPSNVTRLCRAIDSVGIDPHDHQPWQQIVWYDSGVGTNTTAAGPLRNFTEGALGQGLEGNIVEAYHFCALNWNPGDQILCFGFSRGAYTARAIAGLISDIGICPPHQIANFPALWKVYKKTPPGERFYGSDAYYEFFRGRRSSFGGEEGEEEEDGAEEEEGFWESAAEWASTPESRQVQVVGVFDTVGNLGFPELFGHELPAWLTWTDKPEWHNVGLSPNIKNAFQALALDEHRRLFRPAMFFVPAPKNITESQSRAIKQEARNATREWLKLVRSGAASTEELHAAEKRRNAAARELLEWEDSQKEPSQLTQVWFPGFHIHIGGGSNQTLKNKGNMEEMSHIVFAWMLDQIQGYVSLDRTLLQLEEKTREDRLRRINEALEAYQKKERRRQSESWAQWLWHTGQGLITAVQHPLTPSGDAPPAYRQIRQYGWATGDLPDSFDFTYRLNGSLARRPRRYFRDVRTNEVLGHTCECIHPTVGFRLKHVPGYRPAGLRDGQYARQLRADGQGYEYCFRYPGEKDEEVLPEWEMARDELSWERWVVKGKEASAYVDSLFY

SEQ ID NO: 19 - DNA, 1688 bp, Aspergillus japonicus SAITO IFO 4060
  promoter (1)..(363)
  misc_feature (364)..(1488): orf 11 easG
cgttaactgatattgaaggagcattttttgggcttggctggagctagtggaggtcaacaatgaatgcctattttggtttagtcgtccaggcggtgagcacaaaatttgtgtcgtttgacaagatggttcatttaggcaactggtcagatcagccccacttgtagcagtagcggcggcgctcgaagtgtgactcttattagcagacaggaacgaggacattattatcatctgctgcttggtgcacgataacttggtgcgtttgtcaagcaaggtaagtggacgacccggtcataccttcttaagttcgcccttcctccctttatttcagattcaatctgacttacctattctacctaagcattcatgaccatcctcgtcctcggcggccgcggcaagaccgccagccgcctctcagccctcctgcacgaagcccagatccccttcgtcgtgggcacctcctccacggccccgatcgccacctaccgaaccgcccacttcgactggctgaacaaagacacctggaccaacgcgtgggaccaggccccggagcccatcaccgctatctacctcgtaggcgcccacgccccggagttcgtgcacccgatgttcgagttcatcgacctggcgcacagccggggcgtgcggcggtacgcgctcgtcagcgcgagcaacaccgaaaagggggatccgatgatgggggaggcgcacgcgcacctggactcgaaagagggggtcaagtatgtggtgttgcggccgacgtggtttatgggtgcgttgtctggcttttcccttgccgtttcctcgcgtttccctctgagaaggggattggcgtgttttcctatctcgaatactttccccctgcccctaacccccaaatcccaaagtcaggactaattatggagtgaaacagaaaaccacatcgaagacccccacctcagctggatcaagacggagaacaagatctactcggccacgggcccgggcaagatccccttcgtctcggcggatgatatcgcccgcgtcgccttccacgccctgacgcagccgctcacccagaaggagcagcagtacatggttctgggcccggatctgctgtcctatgctgatgtttgtttcttcccaccttccaccccccacctcccatctttccctatctcctaccacggctatccgattcccagataagggtatgctcatgacgagaatgcgatgatactaatgctggagggaaaacagatggccgagatcctcaccgacctcctgggccggaagattacgcacgtcaccctgcccgccgagaagttcggccagttcctggtcgacacggccgggttgcccccggactttgcggagatgctggcgacgatggaggcggcggtggcgacgggggcggaggagcgggtcagccaagatgtcgagaaggtgaccgggcggccgccgcggcggtttgtggattacgcgagggagaatcgcgagcattggttgtgatttcgctggtggggtttgttcgggatgatgtttggtgggagttgggaattggcacttggtctgcagtgcaaaggttcactcagtactcagtatatcctagagtctcaaagtgttgaagggcgggctctggagcctgcggtgaataaaaccgagacttggtgccatacgcttgctaggtcatcgatctcgattgagatagg

SEQ ID NO: 20 - DNA, 1802 bp, Aspergillus japonicus SAITO IFO 4060
  promoter (1)..(363)
  misc_feature (364)..(1603): orf12
cgttaactgatattgaaggagcattttttgggcttggctggagctagtggaggtcaacaatgaatgcctattttggtttagtcgtccaggcggtgagcacaaaatttgtgtcgtttgacaagatggttcatttaggcaactggtcagatcagccccacttgtagcagtagcggcggcgctcgaagtgtgactcttattagcagacaggaacgaggacattattatcatctgctgcttggtgcacgataacttggtgcgtttgtcaagcaaggtaagtggacgacccggtcataccttcttaagttcgcccttcctccctttatttcagattcaatctgacttacctattctacctaagcattcatgacgatcgacccctcctcccccggggcgcccgcctcgaagctcttccaacccatgaagataggcctatgtcacctccagcaccgcctcatcatggcccccaccacccgctaccgcgccgacagcaccgcgacgcccctccccttcgtgcaagaatactacgcccaacgcgccgccgttccggggacactcctgatcaccgaagccaccgacatctccccgcaggcggccggagaaccgcacatcccaggcctgtacagcgcaacccagatcgccgcgtggcgggggatcgtctccgccgtgcaccgccggggctgctacatcttctgccagctgtgggcgaccggacgggcggcggaccccgccctgttgaaggagaaggggttccctctcgtctcgagcagcgatgtgcccatcgagactgaatcgcagcagccgcagccgctgacgcaggccgagatcgaggggtacatcgcggacttcgtgacggcggcaagggtagcggtccacgaggtggggttcgatggggtggagctgcacggcgccaacggatacctcatcgatcagttcacgcagtggccgtgcaaccagcggacggattgctggggcgggagcatcgagaaccgcgcgcggtttgcgctggcggtcactcgcgcggtggtggatgccgtcggggcggagcgcgtcggggttaagctgtcgccctggagtcagtacgccgggatgggggtgatggctgatttggtgccgcagtttgagttcttgatcaaaaggttacgggaggagtacaatgatggtcctgggctggcgtacttgcatctggcgaattcaaggtggttggatgaggagacttcccatccggatccgcatcatgaggtcttcgtgcgcgcgtggagggggtcgggcagggatggtattgaggcgaagaaagaggcgcctccggtgatcctggccggcggctatgatgctgtctcggcgcccgaggtcgctgataaggtgttcaaggaagaggacaatgtcgccgtcgcgtttgggcgatattttatctcgacgcccgacctgccgttccgcatgcaacggggcattccattgcagaagtatgatcgcgcctcgttctatacctcgcttttgaaagaaggttatctggattatccgtacagtgcggagtatgttgcggagtttgggcagaggctgaagaagaaggaggcggcggcgacagggtcgatatgtcagggttgaatggatagggtgggatcgtctgcgtatgttgcgagagcctggcgtgaaaccttttaagcaaagtagattatagaaatagatcggctacgtagggaggcccactgtgaaaaatcatgtaacgcgagcttaatgatagagagaagcacaaaacatcgtctcccagggtgatcagccatgggtgatagatacagtcgagataa

SEQ ID NO: 21 - DNA, 1389 bp, Aspergillus japonicus SAITO IFO 4060
  promoter (1)..(363)
  misc_feature (364)..(1249): orf13
cgttaactgatattgaaggagcattttttgggcttggctggagctagtggaggtcaacaatgaatgcctattttggtttagtcgtccaggcggtgagcacaaaatttgtgtcgtttgacaagatggttcatttaggcaactggtcagatcagccccacttgtagcagtagcggcggcgctcgaagtgtgactcttattagcagacaggaacgaggacattattatcatctgctgcttggtgcacgataacttggtgcgtttgtcaagcaaggtaagtggacgacccggtcataccttcttaagttcgcccttcctccctttatttcagattcaatctgacttacctattctacctaagcattcatgaccgtcgtcaactcgcgcatctttgcagtcaccggcggcgcctccggcattggcgcggccacctgccgcctcctcgccgcgcgcggcgcagcggtcatctgcatcggggacatctcgcccaagggcttcgagagcgtcaccagcgacatccaggccctcaacccggccaccaaggtgcactgcacccggctggacgtctcctcgtccgccgaggttgacgcctggctacagaccatcgtgcagaccttcggggacttgcacgggctggccaacgtcgcgggcatcgcccagccggcgggcgcccgccaggccccggccctgctcgaggagacggacgaggaatggcagcgcgtgttcaaggtcaacctggacggggtcttctactgcacgcgcgcaggtatccgcgcgatgaaggcccttccggggcccaaggaccgcgccatcgtcaacatcggtagcatcgccgcgtttattcacttgccggatgcctacgcctacgggacctccaagggcgcctgcgcgtacttcaccagctgtgcggccatggatgcctggcccctggggattcggatcaacagtgtctcgccgggtaagtgtctttcgtgctgtttattgtatagatctatggacagaggagaggaggatgaccctttggcgaggggggaaagctaacattgaggaaacaggtatcaccgacaccccggctctgccgcagttcatgccccaggcgaagtccatcgatgaggtcaaggaagcgtacatcaaggaggggttccctatcattcagcctgaggatgtggccaggacgattgtctggctgctcagcgaggattcccgcccggtgtatggggccaatatcaatgttggggctgctactccttgagatgctagatgctgtttagctagcttgagaggaggtgtttatatctagagacttggtgttctccatcttaatgacagccaaggttgagcttcaagtcggattcgaaatgtagtagggttggtttatctcgactgtatcta

SEQ ID NO: 22 - DNA, 1445 bp, Aspergillus japonicus SAITO IFO 4060
  promoter (1)..(363)
  misc_feature (364)..(1245): orf14
cgttaactgatattgaaggagcattttttgggcttggctggagctagtggaggtcaacaatgaatgcctattttggtttagtcgtccaggcggtgagcacaaaatttgtgtcgtttgacaagatggttcatttaggcaactggtcagatcagccccacttgtagcagtagcggcggcgctcgaagtgtgactcttattagcagacaggaacgaggacattattatcatctgctgcttggtgcacgataacttggtgcgtttgtcaagcaaggtaagtggacgacccggtcataccttcttaagttcgcccttcctccctttatttcagattcaatctgacttacctattctacctaagcattcatgaccatcaccgagacctccaagccgacgctgcgccgcatcccccggagcgccggcgacgaggccatcttccaagtcctgcaggaagacggagtcgtcgtgatcgaaggattcatgagcgcggaccaggtccgccgcttcaacggggagatcgacccgcacatgaagcagtgggaactgggccagaagtcgtaccaggagagctacctggcggggatgcgacagctgagcagcctgccgctgttcagcaagctcttccgggacgagctgatgaacgacgagctcctgcacgggctgtgcaagcggctgttcgggcccgagtccggcgactactggctgacgacctcctcggtgctggagacagagccgggctaccacgggcaggagctgcaccgcgagcacgacgggatccccatctgcaccaccctgggccgccagagccccgagtccatgctcaacttcctcaccgcgctgaccgacttcaccgcggagaacggcgccacccgcgtcctgccgggcagtcacctctgggaggacttcagcgccccgccccccaaggccgacaccgccatccccgccgtcatgaaccccggcgatgccgttctcttcaccggcaagaccctgcacggcgccggcaagaacaacaccaccgacttcctgcgccgcggcttccccctgatcatgcagtcgtgccagttcacccctgtcgaggcctcggtggccctgccccgtgagctggtcgagaccatgacccccctggcccagaagatggtcggctggcggaccgtctccgctaagggggtggatatctggacgtatgatctgaaggatctggcgacaggaattgatctcaagtcgaaccaggtggcgaaaaaggcgtgagtgggagacttgaccgcaaaataggaaccagtccgttgtcttaggtcagccaaggaaatgtgaaaggaaatctatatcataaatttgttccaaccatttgttccagactgtagacaagaaacaagattgttcgcgagtatgccgtgtgaaccaacatagtacaacttataccctgcctgctacagccttagcatgccatt

SEQ ID NO: 23 - DNA, 2217 bp, Aspergillus japonicus SAITO IFO 4060
  promoter (1)..(363)
  misc_feature (364)..(1967): orf15
cgttaactgatattgaaggagcattttttgggcttggctggagctagtggaggtcaacaatgaatgcctattttggtttagtcgtccaggcggtgagcacaaaatttgtgtcgtttgacaagatggttcatttaggcaactggtcagatcagccccacttgtagcagtagcggcggcgctcgaagtgtgactcttattagcagacaggaacgaggacattattatcatctgctgcttggtgcacgataacttggtgcgtttgtcaagcaaggtaagtggacgacccggtcataccttcttaagttcgcccttcctccctttatttcagattcaatctgacttacctattctacctaagcattcatgactgctgggcaaggcatcaagacgggcaatgcctctgactgtgaagtttaccgcacgctgagcgtcgccctcgactttgccaaccaggatgaggagctctggtggcacagcaccgcgcccatgtttgctcagatgctgcagtccaccaactacaacctccatgctcagtacaagcacctgctcatctacaagaaaaacgtcatccctttcctcggggtttatcccacaaatgacaagccccggtggctaagcatcctgacccgatacggcaccccctttgagctgagcctgaactgctccggcccactggtgcgctacacctacgagcccatcaacgcggccacggggacggcgcgcgacccgttcaacacccacgctgtctgggacagtctggaacagctgatggcgctgcagagcgggattgacctggacctcttccgccacttcaagaacgacctcaccctcagcgctgaggaatctgagtacctgtacaagaacaacctggtgggcgagcagatccggactcagaacaaactggcgctggatctccaggacggcgagttcgtggtcaagacctacatctacccggccctgaagtcgctcgccacgggcaggtccatccacgagctggtcttcgggtctgccttccgctggtccaagcagtacccggagctccgcaagcccctggacacactggagcagtacgtctactcgcggggccccagcagcacggccagcccgcgtctcctctcctgcgacctgatcgaccccaccaagtcccgcatcaagatctacctgctggagcggatggtcaccctggaggccctggaggacctctggaccatggggggcgagcgcaccgacgcctcgaccctggcggggctcgagatgatccgcgagctgtgggagctgatccgcctgcccgcgggcttgcagtcgtatccggccccgtatctgcccatcgggaccatcccggacgagcagctgcccctgatggccaactacaccatccaccacgacgaccccgttccggagccgcaggtctacttcaccaccttcggccgcaacgacatgcagatcgccgacgcgctggccaccttcttcgagcgccgcggctggcacgagatggcgcgccagtacaaggccgagctgtgcagccactacccgcacgccgaccacgagaccctgaactacctgcacgcctacatctccttctcctaccgcaagaacaaaccctacctcagcgtgtatctgcagtcgctggagacgggcgactgggtcacttgtaagctctccccttccgtgttcctttcctgcacacttccccaccgtggatacatccctgatgatctgacctgttcgcgcagcatccttcaactctgtccatgtggaccccggcctctcagccaccgtccaagagctgtccaagctgaccaagaccgccgggaccaccgttcgcgagacgaaactcccactcaccccggatggatcggagcccggcgtcatcacccagtactaggaaatcatctcgttggaccggatttgccagcttcccgccggggagttgcaagtgctttgtatgtaggtaggggaactagatgaaaaagagcaatcgaccggcgagggtcacacgcccggctctgcatgccgtttgccagtgattgtatgtagtaatgatagcctcatgagacagccatttagcttgggcttaatccagttggagtagaaccattgaagttaagttacttaaattcacctccccagtgcttgctggtgtagactttgaccttcgggtgcatggtgtaggacttggctagatgtctccgtgaaatgaacaatctttgactttaa

SEQ ID NO: 24 - DNA, 2606 bp, Aspergillus japonicus SAITO IFO 4060
  promoter (1)..(363)
  misc_feature (364)..(2356): orf16
cgttaactgatattgaaggagcattttttgggcttggctggagctagtggaggtcaacaatgaatgcctattttggtttagtcgtccaggcggtgagcacaaaatttgtgtcgtttgacaagatggttcatttaggcaactggtcagatcagccccacttgtagcagtagcggcggcgctcgaagtgtgactcttattagcagacaggaacgaggacattattatcatctgctgcttggtgcacgataacttggtgcgtttgtcaagcaaggtaagtggacgacccggtcataccttcttaagttcgcccttcctccctttatttcagattcaatctgacttacctattctacctaagcattcatgggtcagtcacgagggatcttgggaggcgtgaggcaacttatcctggttattctggttggggcatacctcagccggctttcagcggtggatgacgaccgccacgactgtcgatgtcggccgggggagccatgctggcccacggtcagccactggagtactctgaacgagtccatcggcggtgctctggctcatgtcaaacccatcgctcacgtctgtcaccagtctggtcaggactcgtcggcttgcgagcaggtcttgcaagagagtcttgacagcaagtggcgggcgtcgcacacgggttagctcattccacctccctgaattcatctgacacacgggctcatccatcccacacaggcgcattgcaagactgggtctgggaaggtggcgcggaatccaaccagacatgttactacctgcggtccggtccggctggatcatgccaccagggccggattcccctctactccgcggctgtcaagtcggccagcgacgtgcagaaggtcgtggacttcactcgtcaacacaatttacgattagtgatccgcaacaccggccacgatgggagcggccgatccagtggaccggactctgtcgagatccacacccaccatctgaacagcgtgcaatatcaccccaacttccgccccgctggatcttctgagcgccagtcggctccaggacagccagcggtgacagtaggcgccggaatcctgcttggggacctctacgcccgcggcgcctcggagggatggatcgtagtgggcggggagtgtcccaccgtcggggcggccggcggattcctccagggcggaggagtatcgagttggctgagctacgcgcatggattggcagtggataatgtgcttgagtatgaagttgtgacggccaaggtgcgtgcgggttcatagcttccctccctccgcgctctaaccctaactaaccctccgcaaatgtccagggagagattgtcatcgccaacgcccatcaaaaccccgatctcttctgggcactgcgcgggggaggaggaggcacctttggcgtcgtgacccaggccacgcttcaagtccaccccgacctccccgtcagcgtcgccgacgccgtggtgacgggctcccgcgccgacgcgaccttctggtcccacggcgtcgccgccctcctgcgagcgctacaattcctcaacaaccacggcacggcaggccagttcatcctccgcaacaccgacgacgacacggtgcaagccagtctgacgatgtacttctcgaacctgaccgtccccgccgtcgccgacgagcgcatggacccgctccgccgcgccctcgaacacaacggacacccctaccagctcacctcgcggttcctgccccagatcagcagcaccttccggcacaccgcggatcgctacccggaggactacggaatcctgatgggctcggtcctggtgtcgctggacctgttcaattccgcgacgggccccgcggcgctggcgcagcacttcgcgcggctcccgatgacccccgacgatctcctgttcaccagcaacctgggcgggcgagtgtcctccctcaaccgcgacccggccagcacagccatgcatcccggctggcgtgacgccgcgcagttgctcaacttcgtccgcggggtcggggccccttctctcgcggctaaggccaccgccctccacgagctgcagacggtccagatggcgaggctgtatgagattgaaccggccttccagatctcgtaccgcaacctgggagatccctcggagcgccgcagtcgggaggtctattgggggtcgaactacgcgcggttagtggaggtcaagcggaggtgggatccggagggactgttcttcagcaagctggggattgatggtgatgcatgggacgcggaaggaatgtgtcgtcagacgcgccaaggggcgtggaatgtggcggtcaaatgggtccagagcttccttgggtctttgtctgcttctgtggtgtagttcattctctcttgatcgattggggaaatgcagttcgacatgataatattgttgtcgggatctcatgtaggataggtcgcctgcgattggagcggtcagattgcaagtgaccgtacgtgcagctgctgacctgccctgccacatttctaagcttggaaggggcagcaagacaggggggttgcactcggggggggctactccttgctggcggcgggtacattcttcgccagagcatgcaggataatgtggc

SEQ ID NO: 25 - DNA, 2224 bp, Aspergillus japonicus SAITO IFO 4060
  promoter (1)..(363)
  promoter (1)..(363)
  misc_feature (364)..(1974): orf17
cgttaactgatattgaaggagcattttttgggcttggctggagctagtggaggtcaacaatgaatgcctattttggtttagtcgtccaggcggtgagcacaaaatttgtgtcgtttgacaagatggttcatttaggcaactggtcagatcagccccacttgtagcagtagcggcggcgctcgaagtgtgactcttattagcagacaggaacgaggacattattatcatctgctgcttggtgcacgataacttggtgcgtttgtcaagcaaggtaagtggacgacccggtcataccttcttaagttcgcccttcctccctttatttcagattcaatctgacttacctattctacctaagcattcatggcgcccgatagcaagccgacctacaccacggcaaatggctgtcccttccacaagcctgagggtcctccggcggatggaaggacgctggcactcagcgaccaccatctggtcgataccctcgcccacttcaaccgggagaagatccccgagcgagtggtgcatgctaaaggagcgggagcctatggcgaatttgaggtatgaggcagcatatcctggtacagacgagggaaggaaccacgcccaagctgagttgctgacccggaccgtccattccaggtgacggccgacatctcggatatctgcgatatcgacatgctggtgggagtgggcaagaagaccccctgtgtgacgcggttctcgacgactggcctcgaacgcgggtccaacgagggaatgcgcgacctgaaaggcatggcctgcaagttctacaccaccgagggcaactgggactgggtgatgctcaacttccccttcttcttcatccgcgaccccgtcaagttccccagcctgatgcacgcccagcgccgggatccgcagaccaacctgctgaaccccaacatgtactgggactgggtgaccaacaaccacgagtcgctgcacatggtcctgctgcagttcagcgacttcgggaccatgttcaactggcggtccatgagcggctacatggcccacgcgtacaaatgggtgaagcccgacgggtcgttcaaatacgtccacatcttcctctcctccgatcgcgggcccaacttcaccgacggccagcaggccaagggcacgaacgacctggatccggaccacgccacccgcgacctgtacgaggccatcgagcgcggcgagtatccgacgtggacggcgagcgtgcaggtggtcgaccccaaggacgcgccgaacctggggtacaacatcctggacgtgaccaagcactggaacctgggcacgtaccccaaggacgtgacgctgatcccgcccaaggtcttcggcaagctgacgctcaagcgcaacccggccaactacttcgccgagatcgagcagctggcctactccccctcgaacatggtgcccggggtggccccctcggaggacccgatcctccaggcgcgcatcttcgcctacccggacgcccagcggtaccggctgggcgccaaccaccagcagatccccgtcaaccggtccgcccacaccttcaaccccatcgcgcgcgacggccagggcaccttcgacgccaactacggcgcccacccgggctttctgacccagcagcagcccgtccgcttcgccgagccccgcgagcccgaccccaagtacaacgagtggctcaaggagatccagtccaagtcctggctccagaccaccgagcacgactacaagttcgcccgcgacttctacgaggtcctgccggacttccgcgggcaggagttccaggacaccatggtccagaatatggtcgagtccgtcgcgcagacgcgcgcggagatccaaaagcaggtctacgagacctggaagctggtgagtccagcgctggcggcgcgaatccagaaaggggtggagacgctgctgcagaagagtgaggcgccggcggaggagtctttcatcccggcaagattgtagagtagctagcagggagtgaaaacgggggggggggggggagatgatgagatctgtcggaatggaatgtctcagggggtcaaagtgggaagggcgcgaggtgttggagttggttacggacttgatatgtcctcacttcttgctgtcagcaaggggaaagagacttcgaacaagaaagaaatcgaacttccctttcgggacctttcgggacctttttccatgagatgtgtcttccccagcgcgtaaggtagtc

SEQ ID NO: 26 - DNA, 1730 bp, Aspergillus japonicus SAITO IFO 4060
  promoter (1)..(363)
  misc_feature (364)..(1480): orf18
cgttaactgatattgaaggagcattttttgggcttggctggagctagtggaggtcaacaatgaatgcctattttggtttagtcgtccaggcggtgagcacaaaatttgtgtcgtttgacaagatggttcatttaggcaactggtcagatcagccccacttgtagcagtagcggcggcgctcgaagtgtgactcttattagcagacaggaacgaggacattattatcatctgctgcttggtgcacgataacttggtgcgtttgtcaagcaaggtaagtggacgacccggtcataccttcttaagttcgcccttcctccctttatttcagattcaatctgacttacctattctacctaagcattcatgggcagcatcaatcccccgattttggacatccgccagtcgaccttcgagacctccatcccccaacaagtaaccgagggactcaccaaagaccccaaaaccctgccggcccttcttttctacagcggcgatggcatccagcactggacgcgccactcgtgcgccccggatttctacccccgccgcgaggagatcaacatcctccacagcgaagcggctcacatggcgtatgcaaggcctcgataagccaggacagtgttgtggtggatctgggcagcgcgtatgcaagcacacacaatctacaatcaaagcccagcagaggtgtcggggaaatgaataactaaaccgcaaacccaatgcaccttatagatccctcgataaagtcctccccttcctgcacgccctcgaggcccaacagaagcgcgttcactactacgccctcgacctgagctacccggtgctcgcctcgacgctgtccgagatccccgtggcgcagttccagtatgtccagatcgccgcgctgcacggcaccttcgaggacgggctgcagtggctgcacgactcgcccgaggtccgcgagcgcccccaccacctgctgctgttcggcctcacgatcggcaacttctcacggcccaacgccgccgcctttttacggaatatcgccgaccgggcgctggcggcgtcccccgcggagagctccatcctcctcaccctggacagctgcaagatgccgaccaaggtgctgcgcgcgtacacggcggaaggggtggtgcctttcgcgctgacctcgttgagctatgccaatcagctcttctggccgggtggggagggaagggtgtttgatgaagaggaatggtatttccacagtgagtggaacttcgcgctggggcgccacgaggcggcgctcatcccccgggatcgcgatatcaccctcgggccgccgttggagcaggtcgtcgtgcgccggggtgagaaggtgaggtttggctgcagctacaagtatagtgctgcggagcgggagcatttgttcgctggggcggggttggagaatgtggcttcctggtcggccaagggctgtgatgttgctttttatcagttgaagatgcggcccgagtgagggcttgacgtggtcatttccgggtgggattgaacagggttttggtgcagagtacaccttgttactaggaagtttcgtcaggaactgcagtttacccgttcgtatttaatctatgagagcacattaagcgggaaggagattggtattcgacaacaacaacattgtgtctttctttttgtatttggattggaacacatatccggtagccacactgcgctgacggccatcacaacaggaaaatgttgttgca

SEQ ID NO: 27 - DNA, 2450 bp, Aspergillus japonicus SAITO IFO 4060
  promoter (1)..(363)
  misc_feature (364)..(2200): orf19
cgttaactgatattgaaggagcattttttgggcttggctggagctagtggaggtcaacaatgaatgcctattttggtttagtcgtccaggcggtgagcacaaaatttgtgtcgtttgacaagatggttcatttaggcaactggtcagatcagccccacttgtagcagtagcggcggcgctcgaagtgtgactcttattagcagacaggaacgaggacattattatcatctgctgcttggtgcacgataacttggtgcgtttgtcaagcaaggtaagtggacgacccggtcataccttcttaagttcgcccttcctccctttatttcagattcaatctgacttacctattctacctaagcattcatgcaccgccccctcacccccacccccaagccccaacccgcgcgcaagcgcctcctcatctgctgcgacggcacctggcaatccgccgtcgacggccaagagcgcatcccctcgaacgtgacgcggctctgccgcgccatcgacagcgtggggatcgacccgcacgaccaccagccctggcagcagatcgtctggtacgactccggggtgggcaccaacaccacggcggcgggcccgctgcgcaacttcaccgagggcgccctgggccaagggctggagggcaacatcgtcgaggcctaccacttctgcgccctgaactggaacccgggcgaccagatcctgtgctttgggttctcgcgcggcgcctacaccgcgcgcgccatcgccggcctcatctcggacatcggcatctgcccgccgcaccagatcgcgaatttcccggcgctgtggaaggtgtataagaagacgccgcccggggagcggttctatggcagtgatgcgtactatgagtttttccgggggcgccgatcctccttcggcggagaagagggggaagaggaggaggatggggcagaagaagaggagggtttttgggagtcggccgcagaatgggcatcgacccccgagtcacggcaggtgcaagtggtcggggtcttcgataccgtcggcaatctgggcttccccgagctgttcggccatgagctccctgcctggctgacctggactgataagcctgaatggcacaatgtcggtctttcgcctagtaagcaatccctccaggctatcattggctcaggatgagacgatactgatcccaaacccagacatcaagaatgcattccaggcgctggcgttggacgagcaccgccgactgttccgcccggcgatgttcttcgtcccggcgccgaagaatatcacggagagccaatcgcgagcgatcaagcaggaggcacggaacgccacccgcgagtggctaaaactcgtaagaagcggtgccgccagcaccgaggagttacacgcagccgagaaacgacgcaacgcggctgcccgtgagcttttggaatgggaggacagccagaaggagccgtcccagctgacgcaggtctggtttccggggttccacattcacatcgggggcggatccaaccagaccctcaagaacaagggcaacatggaagagatgtcccacatcgtcttcgcgtggatgctggaccagatccaggggtacgtctccctcgaccgcacgctcctccagctcgaggaaaagactcgagaggatcgtctgcggcggatcaacgaggcgctggaggcgtaccagaagaaagaacgtcgccggcagtccgagtcctgggcccagtggctgtggcacacagggcagggtctgattaccgccgtgcaacacccgctcaccccgtcgggcgatgctcccccggcctatcgccagatccgtcagtacggctgggccaccggcgacttgcccgacagcttcgacttcacctaccgcctgaacgggtcgctggcgcgcaggccgcgtcgctattttcgggatgtccgcacgaatgaggttctcgggcatacttgcgagtgcatccatcccacggtggggtttcgactgaagcacgtgccgggatatcgtccggctgggctgcgtgatggccagtatgctcgtcagcttagggctgacggccagggctatgagtattgcttcaggtaccccggtgagaaggatgaagaggtcttgccggagtgggagatggctagagatgagctctcctgggaacggtgggttgtcaagggaaaggaggcgtcggcgtatgtggatagcttattctattgacctgattgttaatcgtatacgagcgagtgggtgagcccactggtatgagtgtttacatcacctccagttgcttgtacaggtgtctattggcgtgttcagcataacaatatacctaggtacctgccgggtacccagggaagatctcggagattatgtagttaggtagttatcagaacacaagtagccctagcctcaccatattcagacctcattcatcagaaagatttctaagattgcggctgccacacat

SEQ ID NO: 28 - DNA, 3941 bp, Artificial Sequence
  pHS1
cacctgacgcgccctgtagcggcgcattaagcgcggcgggtgtggtggttacgcgcagcgtgaccgctacacttgccagcgccctagcgcccgctcctttcgctttcttcccttcctttctcgccacgttcgccggctttccccgtcaagctctaaatcgggggctccctttagggttccgatttagtgctttacggcacctcgaccccaaaaaacttgattagggtgatggttcacgtagtgggccatcgccctgatagacggtttttcgccctttgacgttggagtccacgttctttaatagtggactcttgttccaaactggaacaacactcaaccctatctcggtctattcttttgatttataagggattttgccgatttcggcctattggttaaaaaatgagctgatttaacaaaaatttaacgcgaattttaacaaaatattaacgcttacaatttccattcgccattcaggctgcgcaactgttgggaagggcgatcggtgcgggcctcttcgctattacgccagctggcgaaagggggatgtgctgcaaggcgattaagttgggtaacgccagggttttcccagtcacgacgttgtaaaacgacggccagtgaattgtaatacgactcactatagggcgaattatttaaatgggtaccgggccccccctcgaggtcgacggtatcgataagcttgatatcgaattcctgcagcccgggggatccactagttctagagaataggaacttccgttaactgatattgaaggagcattttttgggcttggctggagctagtggaggtcaacaatgaatgcctattttggtttagtcgtccaggcggtgagcacaaaatttgtgtcgtttgacaagatggttcatttaggcaactggtcagatcagccccacttgtagcagtagcggcggcgctcgaagtgtgactcttattagcagacaggaacgaggacattattatcatctgctgcttggtgcacgataacttggtgcgtttgtcaagcaaggtaagtggacgacccggtcataccttcttaagttcgcccttcctccctttatttcagattcaatctgacttacctattctacctaagcattcatggccaccctcgatgacacggcttaccgctaccgtaccagcgtccccggcgacgccgaagccatcgaggccctggatggctctttcaccacggataccgtctttcgcgttaccgctaccggtgacggcttcacgctccgtgaggtgcccgtcgaccctcccctgaccaaggttttccctgatgacgaatctgacgatgaatccgacgatggcgaggacggcgatcccgactctcgcacgttcgtcgcttacggcgatgacggtgacctggccggctttgtcgttgtgtcctattccggttggaaccgtcgcctgaccgtcgaagacatcgaggttgcccccgagcatcgcggccacggtgtcggccgcgctctcatgggcctcgccacggagttcgcccgtgagcgcggcgccggtcacctctggctggaggttaccaatgtcaacgctcccgccattcacgcctaccgtcgcatgggctttaccctctgcggcctggataccgctctctacgatggcaccgcctccgatggcgagcaggccctctatatgagcatgccttgcccctgagaagttcctatactttatttaaatctagagcggccgccaccgcggtggagctccagcttttgttccctttagtgagggttaatttcgagcttggcgtaatcatggtcatagctgtttcctgtgtgaaattgttatccgctcacaattccacacaacatacgagccggaagcataaagtgtaaagcctggggtgcctaatgagtgagctaactcacattaattgcgttgcgctcactgcccgctttccagtcgggaaacctgtcgtgccagctgcattaatgaatcggccaacgcgcggggagaggcggtttgcgtattgggcgctcttccgcttcctcgctcactgactcgctgcgctcggtcgttcggctgcggcgagcggtatcagctcactcaaaggcggtaatacggttatccacagaatcaggggataacgcaggaaagaacatgtgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcatagctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaaggacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggtctgacgctcagtggaacgaaaactcacgttaagggattttggtcatgagattatcaaaaaggatcttcacctagatccttttaaattaaaaatgaagttttaaatcaatctaaagtatatatgagtaaacttggtctgacagttaccaatgcttaatcagtgaggcacctatctcagcgatctgtctatttcgttcatccatagttgcctgactccccgtcgtgtagataactacgatacgggagggcttaccatctggccccagtgctgcaatgataccgcgagacccacgctcaccggctccagatttatcagcaataaaccagccagccggaagggccgagcgcagaagtggtcctgcaactttatccgcctccatccagtctattaattgttgccgggaagctagagtaagtagttcgccagttaatagtttgcgcaacgttgttgccattgctacaggcatcgtggtgtcacgctcgtcgtttggtatggcttcattcagctccggttcccaacgatcaaggcgagttacatgatcccccatgttgtgcaaaaaagcggttagctccttcggtcctccgatcgttgtcagaagtaagttggccgcagtgttatcactcatggttatggcagcactgcataattctcttactgtcatgccatccgtaagatgcttttctgtgactggtgagtactcaaccaagtcattctgagaatagtgtatgcggcgaccgagttgctcttgcccggcgtcaatacgggataataccgcgccacatagcagaactttaaaagtgctcatcattggaaaacgttcttcggggcgaaaactctcaaggatcttaccgctgttgagatccagttcgatgtaacccactcgtgcacccaactgatcttcagcatcttttactttcaccagcgtttctgggtgagcaaaaacaggaaggcaaaatgccgcaaaaaagggaataagggcgacacggaaatgttgaatactcatactcttcctttttcaatattattgaagcatttatcagggttattgtctcatgagcggatacatatttgaatgtatttagaaaaataaacaaataggggttccgcgcacatttccccgaaaagtgc

SEQ ID NO: 29 - DNA, 2974 bp, Artificial Sequence
  pHS1deltaNat1
cacctgacgcgccctgtagcggcgcattaagcgcggcgggtgtggtggttacgcgcagcgtgaccgctacacttgccagcgccctagcgcccgctcctttcgctttcttcccttcctttctcgccacgttcgccggctttccccgtcaagctctaaatcgggggctccctttagggttccgatttagtgctttacggcacctcgaccccaaaaaacttgattagggtgatggttcacgtagtgggccatcgccctgatagacggtttttcgccctttgacgttggagtccacgttctttaatagtggactcttgttccaaactggaacaacactcaaccctatctcggtctattcttttgatttataagggattttgccgatttcggcctattggttaaaaaatgagctgatttaacaaaaatttaacgcgaattttaacaaaatattaacgcttacaatttccattcgccattcaggctgcgcaactgttgggaagggcgatcggtgcgggcctcttcgctattacgccagctggcgaaagggggatgtgctgcaaggcgattaagttgggtaacgccagggttttcccagtcacgacgttgtaaaacgacggccagtgaattgtaatacgactcactatagggcgaattatttaaatgggtaccgggccccccctcgaggtcgacggtatcgataagcttgatatcgaattcctgcagcccgggggatccactagttctagaatttaaatgcggccgccaccgcggtggagctccagcttttgttccctttagtgagggttaatttcgagcttggcgtaatcatggtcatagctgtttcctgtgtgaaattgttatccgctcacaattccacacaacatacgagccggaagcataaagtgtaaagcctggggtgcctaatgagtgagctaactcacattaattgcgttgcgctcactgcccgctttccagtcgggaaacctgtcgtgccagctgcattaatgaatcggccaacgcgcggggagaggcggtttgcgtattgggcgctcttccgcttcctcgctcactgactcgctgcgctcggtcgttcggctgcggcgagcggtatcagctcactcaaaggcggtaatacggttatccacagaatcaggggataacgcaggaaagaacatgtgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcatagctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaaggacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggtctgacgctcagtggaacgaaaactcacgttaagggattttggtcatgagattatcaaaaaggatcttcacctagatccttttaaattaaaaatgaagttttaaatcaatctaaagtatatatgagtaaacttggtctgacagttaccaatgcttaatcagtgaggcacctatctcagcgatctgtctatttcgttcatccatagttgcctgactccccgtcgtgtagataactacgatacgggagggcttaccatctggccccagtgctgcaatgataccgcgagacccacgctcaccggctccagatttatcagcaataaaccagccagccggaagggccgagcgcagaagtggtcctgcaactttatccgcctccatccagtctattaattgttgccgggaagctagagtaagtagttcgccagttaatagtttgcgcaacgttgttgccattgctacaggcatcgtggtgtcacgctcgtcgtttggtatggcttcattcagctccggttcccaacgatcaaggcgagttacatgatcccccatgttgtgcaaaaaagcggttagctccttcggtcctccgatcgttgtcagaagtaagttggccgcagtgttatcactcatggttatggcagcactgcataattctcttactgtcatgccatccgtaagatgcttttctgtgactggtgagtactcaaccaagtcattctgagaatagtgtatgcggcgaccgagttgctcttgcccggcgtcaatacgggataataccgcgccacatagcagaactttaaaagtgctcatcattggaaaacgttcttcggggcgaaaactctcaaggatcttaccgctgttgagatccagttcgatgtaacccactcgtgcacccaactgatcttcagcatcttttactttcaccagcgtttctgggtgagcaaaaacaggaaggcaaaatgccgcaaaaaagggaataagggcgacacggaaatgttgaatactcatactcttcctttttcaatattattgaagcatttatcagggttattgtctcatgagcggatacatatttgaatgtatttagaaaaataaacaaataggggttccgcgcacatttccccgaaaagtgc

SEQ ID NO: 30 - DNA, 19947 bp, Aspergillus japonicus SAITO IFO 4060
  misc_feature (654)..(1778): orf11
atttatttttttattttataccttttctataactgtaggctgtatcacagtgcagacctagtgcaccgcttccacctattctacctgcttgacctatctaacctgtttaatctatttaatctattttatttgttgttttttttaagcttatctacatgatgagtccacaaggtatggagatgtagggctatcattctgccgaagggctgtgcggatgaaccatggtccgggtaaaggtttcggcgcagttcatccacgacctgctcacggcgggcatgttgagatgtctctgaacgtcgtacgtaagtttatcataccctgggaggcatggtgggcgtgtggtatctctaatgactggcaatggggaatccgccgcccgttcccaccactgataataccccgttgcggggtatctgcgcacatagctacgaattctcctctacggtacagtggcctatctcaatcgagatcgatgacctagcaagcgtatggcaccaagtctcggttttattcaccgcaggctccagagcccgcccttcaacactttgagactctaggatatactgagtactgagtgaacctttgcactgcagaccaagtgccaattcccaactcccaccaaacatcatcccgaacaaaccccaccagcgaaatcacaaccaatgctcgcgattctccctcgcgtaatccacaaaccgccgcggcggccgcccggtcaccttctcgacatcttggctgacccgctcctccgcccccgtcgccaccgccgcctccatcgtcgccagcatctccgcaaagtccgggggcaacccggccgtgtcgaccaggaactggccgaacttctcggcgggcagggtgacgtgcgtaatcttccggcccaggaggtcggtgaggatctcggccatctgttttccctccagcattagtatcatcgcattctcgtcatgagcatacccttatctgggaatcggatagccgtggtaggagatagggaaagatgggaggtggggggtggaaggtgggaagaaacaaacatcagcataggacagcagatccgggcccagaaccatgtactgctgctccttctgggtgagcggctgcgtcagggcgtggaaggcgacgcgggcgatatcatccgccgagacgaaggggatcttgcccgggcccgtggccgagtagatcttgttctccgtcttgatccagctgaggtgggggtcttcgatgtggttttctgtttcactccataattagtcctgactttgggatttgggggttaggggcagggggaaagtattcgagataggaaaacacgccaatccccttctcagagggaaacgcgaggaaacggcaagggaaaagccagacaacgcacccataaaccacgtcggccgcaacaccacatacttgaccccctctttcgagtccaggtgcgcgtgcgcctcccccatcatcggatcccccttttcggtgttgctcgcgctgacgagcgcgtaccgccgcacgccccggctgtgcgccaggtcgatgaactcgaacatcgggtgcacgaactccggggcgtgggcgcctacgaggtagatagcggtgatgggctccggggcctggtcccacgcgttggtccaggtgtctttgttcagccagtcgaagtgggcggttcggtaggtggcgatcggggccgtggaggaggtgcccacgacgaaggggatctgggcttcgtgcaggagggctgagaggcggctggcggtcttgccgcggccgccgaggacgaggatggtcattttgtcggagggggttaagaagaaggatagctgtcgagggcgaggaggtaagagccaaggtaggtttgggaggtttatttgctggcgataaggatttataccggatgtatggaaatggtgggccggagaatgagggggaggggagggggcggggtgcggtgaggtgatggcacaattgacaagcacactcaccattcaccattgacatgcaggatcgagactggagcaaatcgtgttcgatgaagattggtctgatgatttcccaactcaatgcgggagaaccttgctttggggacatttctggtgggttgcaagctacgttggtgctgcagctcgaagctcggatgcgaggatgagctgggcagaagggtggggcatgcaggatgcaggatgcatgcatttacggatgtagcgtttcagaattgggccactgcggcaatagggccacgtagtcctttattggtgtacggcggcctaaagaatgaggttgtgagaggcagaggcaagaagaaagtctgcgagaagcgaggagagagccgagttagggcttgggaggggggaggtgcagagcagagggagagagagtgtgcggccacggcggtcaatttaccgccacaaccacaagaaccaaacagtcggctggataaatatgtacaagcgatatcagtccggtcaatggcactaaaatagcccaaccttcggctcttgtccagcttcaattgtatgctgcaatagcgccccgccacccaacaacatgacgatcgacccctcctcccccggggcgcccgcctcgaagctcttccaacccatgaagataggcctatgtcacctccagcaccgcctcatcatggcccccaccacccgctaccgcgccgacagcaccgcgacgcccctccccttcgtgcaagaatactacgcccaacgcgccgccgttccggggacactcctgatcaccgaagccaccgacatctccccgcaggcggccggagaaccgcacatcccaggcctgtacagcgcaacccagatcgccgcgtggcgggggatcgtctccgccgtgcaccgccggggctgctacatcttctgccagctgtgggcgaccggacgggcggcggaccccgccctgttgaaggagaaggggttccctctcgtctcgagcagcgatgtgcccatcgagactgaatcgcagcagccgcagccgctgacgcaggccgagatcgaggggtacatcgcggacttcgtgacggcggcaagggtagcggtccacgaggtggggttcgatggggtggagctgcacggcgccaacggatacctcatcgatcagttcacgcagtggccgtgcaaccagcggacggattgctggggcgggagcatcgagaaccgcgcgcggtttgcgctggcggtcactcgcgcggtggtggatgccgtcggggcggagcgcgtcggggttaagctgtcgccctggagtcagtacgccgggatgggggtgatggctgatttggtgccgcagtttgagttcttgatcaaaaggttacgggaggagtacaatgatggtcctgggctggcgtacttgcatctggcgaattcaaggtggttggatgaggagacttcccatccggatccgcatcatgaggtcttcgtgcgcgcgtggagggggtcgggcagggatggtattgaggcgaagaaagaggcgcctccggtgatcctggccggcggctatgatgctgtctcggcgcccgaggtcgctgataaggtgttcaaggaagaggacaatgtcgccgtcgcgtttgggcgatattttatctcgacgcccgacctgccgttccgcatgcaacggggcattccattgcagaagtatgatcgcgcctcgttctatacctcgcttttgaaagaaggttatctggattatccgtacagtgcggagtatgttgcggagtttgggcagaggctgaagaagaaggaggcggcggcgacagggtcgatatgtcagggttgaatggatagggtgggatcgtctgcgtatgttgcgagagcctggcgtgaaaccttttaagcaaagtagattatagaaatagatcggctacgtagggaggcccactgtgaaaaatcatgtaacgcgagcttaatgatagagagaagcacaaaacatcgtctcccagggtgatcagccatgggtgatagatacagtcgagataaaccaaccctactacatttcgaatccgacttgaagctcaaccttggctgtcattaagatggagaacaccaagtctctagatataaacacctcctctcaagctagctaaacagcatctagcatctcaaggagtagcagccccaacattgatattggccccatacaccgggcgggaatcctcgctgagcagccagacaatcgtcctggccacatcctcaggctgaatgatagggaacccctccttgatgtacgcttccttgacctcatcgatggacttcgcctggggcatgaactgcggcagagccggggtgtcggtgatacctgtttcctcaatgttagctttcccccctcgccaaagggtcatcctcctctcctctgtccatagatctatacaataaacagcacgaaagacacttacccggcgagacactgttgatccgaatccccaggggccaggcatccatggccgcacagctggtgaagtacgcgcaggcgcccttggaggtcccgtaggcgtaggcatccggcaagtgaataaacgcggcgatgctaccgatgttgacgatggcgcggtccttgggccccggaagggccttcatcgcgcggatacctgcgcgcgtgcagtagaagaccccgtccaggttgaccttgaacacgcgctgccattcctcgtccgtctcctcgagcagggccggggcctggcgggcgcccgccggctgggcgatgcccgcgacgttggccagcccgtgcaagtccccgaaggtctgcacgatggtctgtagccaggcgtcaacctcggcggacgaggagacgtccagccgggtgcagtgcaccttggtggccgggttgagggcctggatgtcgctggtgacgctctcgaagcccttgggcgagatgtccccgatgcagatgaccgctgcgccgcgcgcggcgaggaggcggcaggtggccgcgccaatgccggaggcgccgccggtgactgcaaagatgcgcgagttgacgacggtcatgatgctgtcgtgtgaggtggtgagagggggctggggtaggaaggggattgtggaaagaggtttcgtgtaagccattgaacgggcaggcggtttctggctattttaccctctccctatcctgctgttgcccctctcctctacttgagcgcggggaatgcgtcaatgatgatgcttcacgttggcgggcgaggtagcatccacccccttggctccggacctgcggtcggtggttgcgaggagcattttagtctgcggttgacagacgtgaggatgcccgagatctgcagctgccacttgctatgcaatgcagagcagcctggggcgtcgccagcacagaaggcctgatcctcgattgcaatagcggagtagggacgatagagagcagaccgccggtgctcattccgtggatgcagtgatggtatgttgcatcctcgatggcctttttttccattcaaggaatcgtgcacggggcgactttgttgtcgcatgcaatgtacgaacacgtattgcacagcagccgtcgctcattattcgaggtccgatcgatctgaagcaagctgcaaggagacgcatggggccagacagtgggccatatcgcataccaagctgtgtgtctagcagactgtcagtgattgatcaaatgatcccgccacgagagaggaacaaagacagctcgagtagtgtaagagggggggagggaggaagtaaggggaagtagggttaatgccgcaagggggggattgcccctccatcacgtgactagttgtgctggcagcccgacccaaaaagggcaatgatactcacagtgcatgtccatgtcctctgctcagctattttgagtatgtgcatggctgatgcagtgcagcatgcataaatccctcacccacctgactgaaaatcggcctcgtctacaccttcagagcaaatggcacgatcaggcacaatgccatggtctggatggattcccggtgcgcatgccagcgtctgtctcccttgctgccgtactgggcttgtgatacccaaaatgccgcacgggtagccactcatggcctctctccagcttgacaggctgtcgaaatgtgcggtgctcggcttcagacgggggaggaggggcaattgacggccgtgacagatgtatggagtgaaagacatagttccatagacatatgccggtatatatatccagaagaggaccttggtgagacctcttcatccttagaaccttcgatcctaaactacgctcccctccccggcaaagaaaacactcaaaatgaccatcaccgagacctccaagccgacgctgcgccgcatcccccggagcgccggcgacgaggccatcttccaagtcctgcaggaagacggagtcgtcgtgatcgaaggattcatgagcgcggaccaggtccgccgcttcaacggggagatcgacccgcacatgaagcagtgggaactgggccagaagtcgtaccaggagagctacctggcggggatgcgacagctgagcagcctgccgctgttcagcaagctcttccgggacgagctgatgaacgacgagctcctgcacgggctgtgcaagcggctgttcgggcccgagtccggcgactactggctgacgacctcctcggtgctggagacagagccgggctaccacgggcaggagctgcaccgcgagcacgacgggatccccatctgcaccaccctgggccgccagagccccgagtccatgctcaacttcctcaccgcgctgaccgacttcaccgcggagaacggcgccacccgcgtcctgccgggcagtcacctctgggaggacttcagcgccccgccccccaaggccgacaccgccatccccgccgtcatgaaccccggcgatgccgttctcttcaccggcaagaccctgcacggcgccggcaagaacaacaccaccgacttcctgcgccgcggcttccccctgatcatgcagtcgtgccagttcacccctgtcgaggcctcggtggccctgccccgtgagctggtcgagaccatgacccccctggcccagaagatggtcggctggcggaccgtctccgctaagggggtggatatctggacgtatgatctgaaggatctggcgacaggaattgatctcaagtcgaaccaggtggcgaaaaaggcgtgagtgggagacttgaccgcaaaataggaaccagtccgttgtcttaggtcagccaaggaaatgtgaaaggaaatctatatcataaatttgttccaaccatttgttccagactgtagacaagaaacaagattgttcgcgagtatgccgtgtgaaccaacatagtacaacttataccctgcctgctacagccttagcatgccattcacaaaatgaatacccttaggaaagtgcgccgaccgcaaatcaccctatgcaaagcgccatgtccaatcccccaatcccttaaggatgataatagatcgcccagttactgagcacaaagtatttgtgattcttattatgaaagctagatctacgaactggcttataacgttctagctggttattaaattcattacattgatctactagaagtggtaaatagctcttttatataataacctgcctgtaatatataagcacaagatatatatatacctcaacatattctagtgaattattaatattttgaatttgaaaggcaaggagctttactaactagtatagctagcttttaaataacttagatactatactttggttagaataagatatatgagaaataagtattttatgtttcatatatttggataatacttcaagcactaattctatgcagtagttgtatattcctttggtataacaataaattatttaaaagtccataatcgcgaagacataacatctacaaaattaaagtaaatcttcttgtggttgaggttcacaaatatattaaagtcaaagattgttcatttcacggagacatctagccaagtcctacaccatgcacccgaaggtcaaagtctacaccagcaagcactggggaggtgaatttaagtaacttaacttcaatggttctactccaactggattaagcccaagctaaatggctgtctcatgaggctatcattactacatacaatcactggcaaacggcatgcagagccgggcgtgtgaccctcgccggtcgattgctctttttcatctagttcccctacctacatacaaagcacttgcaactccccggcgggaagctggcaaatccggtccaacgagatgatttcctagtactgggtgatgacgccgggctccgatccatccggggtgagtgggagtttcgtctcgcgaacggtggtcccggcggtcttggtcagcttggacagctcttggacggtggctgagaggccggggtccacatggacagagttgaaggatgctgcgcgaacaggtcagatcatcagggatgtatccacggtggggaagtgtgcaggaaaggaacacggaaggggagagcttacaagtgacccagtcgcccgtctccagcgactgcagatacacgctgaggtagggtttgttcttgcggtaggagaaggagatgtaggcgtgcaggtagttcagggtctcgtggtcggcgtgcgggtagtggctgcacagctcggccttgtactggcgcgccatctcgtgccagccgcggcgctcgaagaaggtggccagcgcgtcggcgatctgcatgtcgttgcggccgaaggtggtgaagtagacctgcggctccggaacggggtcgtcgtggtggatggtgtagttggccatcaggggcagctgctcgtccgggatggtcccgatgggcagatacggggccggatacgactgcaagcccgcgggcaggcggatcagctcccacagctcgcggatcatctcgagccccgccagggtcgaggcgtcggtgcgctcgccccccatggtccagaggtcctccagggcctccagggtgaccatccgctccagcaggtagatcttgatgcgggacttggtggggtcgatcaggtcgcaggagaggagacgcgggctggccgtgctgctggggccccgcgagtagacgtactgctccagtgtgtccaggggcttgcggagctccgggtactgcttggaccagcggaaggcagacccgaagaccagctcgtggatggacctgcccgtggcgagcgacttcagggccgggtagatgtaggtcttgaccacgaactcgccgtcctggagatccagcgccagtttgttctgagtccggatctgctcgcccaccaggttgttcttgtacaggtactcagattcctcagcgctgagggtgaggtcgttcttgaagtggcggaagaggtccaggtcaatcccgctctgcagcgccatcagctgttccagactgtcccagacagcgtgggtgttgaacgggtcgcgcgccgtccccgtggccgcgttgatgggctcgtaggtgtagcgcaccagtgggccggagcagttcaggctcagctcaaagggggtgccgtatcgggtcaggatgcttagccaccggggcttgtcatttgtgggataaaccccgaggaaagggatgacgtttttcttgtagatgagcaggtgcttgtactgagcatggaggttgtagttggtggactgcagcatctgagcaaacatgggcgcggtgctgtgccaccagagctcctcatcctggttggcaaagtcgagggcgacgctcagcgtgcggtaaacttcacagtcagaggcattgcccgtcttgatgccttgcccagcagtcatggttttggttttagcgagcggagggtttgaacaaatgatgttgaagtccgttccgttccgttcgtcccttgactgggttggaaactgccgctgggttgtatttataccaggaggaatgcaaaaataggtatgacgctagttgtcgagctgaggaggggggaagaagacacaaagcgttgaagaagacgctctctcgtcacgtgtatttgggatgtatctgctccgagtatctgcagataccaggtgaagttcgaggctcacggactatctccattttcaccgctgtgggaagggtggtccgagacaacaagagctggacgaagggcaggccaaaaatccgctcccaagatgctgtcgtcgcattgggtggcaatcgggcgagatcggcaagagtaatgctagatgcaggaaagacatgctcgcgcaactccgagtgccttgctgctgagcagattagacgagtcggaggaatgcaattgacgtggagaatgactgctattcttatgatcttagacctacagcacacatgtacctacgtgtatgatccctccccttgcacgcccctcttacaccccctcccctcgcacccccttgcttgcctcgagtccttggctggaatatccggacagggcggtgcctcgcgaactggaggcatcctacgctatatctggttggttgcagcctctgcgcaagttgcctttgtaagtccatacgatgatgatgggtcagtcacgagggatcttgggaggcgtgaggcaacttatcctggttattctggttggggcatacctcagccggctttcagcggtggatgacgaccgccacgactgtcgatgtcggccgggggagccatgctggcccacggtcagccactggagtactctgaacgagtccatcggcggtgctctggctcatgtcaaacccatcgctcacgtctgtcaccagtctggtcaggactcgtcggcttgcgagcaggtcttgcaagagagtcttgacagcaagtggcgggcgtcgcacacgggttagctcattccacctccctgaattcatctgacacacgggctcatccatcccacacaggcgcattgcaagactgggtctgggaaggtggcgcggaatccaaccagacatgttactacctgcggtccggtccggctggatcatgccaccagggccggattcccctctactccgcggctgtcaagtcggccagcgacgtgcagaaggtcgtggacttcactcgtcaacacaatttacgattagtgatccgcaacaccggccacgatgggagcggccgatccagtggaccggactctgtcgagatccacacccaccatctgaacagcgtgcaatatcaccccaacttccgccccgctggatcttctgagcgccagtcggctccaggacagccagcggtgacagtaggcgccggaatcctgcttggggacctctacgcccgcggcgcctcggagggatggatcgtagtgggcggggagtgtcccaccgtcggggcggccggcggattcctccagggcggaggagtatcgagttggctgagctacgcgcatggattggcagtggataatgtgcttgagtatgaagttgtgacggccaaggtgcgtgcgggttcatagcttccctccctccgcgctctaaccctaactaaccctccgcaaatgtccagggagagattgtcatcgccaacgcccatcaaaaccccgatctcttctgggcactgcgcgggggaggaggaggcacctttggcgtcgtgacccaggccacgcttcaagtccaccccgacctccccgtcagcgtcgccgacgccgtggtgacgggctcccgcgccgacgcgaccttctggtcccacggcgtcgccgccctcctgcgagcgctacaattcctcaacaaccacggcacggcaggccagttcatcctccgcaacaccgacgacgacacggtgcaagccagtctgacgatgtacttctcgaacctgaccgtccccgccgtcgccgacgagcgcatggacccgctccgccgcgccctcgaacacaacggacacccctaccagctcacctcgcggttcctgccccagatcagcagcaccttccggcacaccgcggatcgctacccggaggactacggaatcctgatgggctcggtcctggtgtcgctggacctgttcaattccgcgacgggccccgcggcgctggcgcagcacttcgcgcggctcccgatgacccccgacgatctcctgttcaccagcaacctgggcgggcgagtgtcctccctcaaccgcgacccggccagcacagccatgcatcccggctggcgtgacgccgcgcagttgctcaacttcgtccgcggggtcggggccccttctctcgcggctaaggccaccgccctccacgagctgcagacggtccagatggcgaggctgtatgagattgaaccggccttccagatctcgtaccgcaacctgggagatccctcggagcgccgcagtcgggaggtctattgggggtcgaactacgcgcggttagtggaggtcaagcggaggtgggatccggagggactgttcttcagcaagctggggattgatggtgatgcatgggacgcggaaggaatgtgtcgtcagacgcgccaaggggcgtggaatgtggcggtcaaatgggtccagagcttccttgggtctttgtctgcttctgtggtgtagttcattctctcttgatcgattggggaaatgcagttcgacatgataatattgttgtcgggatctcatgtaggataggtcgcctgcgattggagcggtcagattgcaagtgaccgtacgtgcagctgctgacctgccctgccacatttctaagcttggaaggggcagcaagacaggggggttgcactcggggggggctactccttgctggcggcgggtacattcttcgccagagcatgcaggataatgtggcagtgcgagttgacaacggagggagatgacatgacaccaaatccccggtgaggggttttggggtataggtcacaagtcgcaggatgcgagtcactgcggcaaggcaagcaaacagggcggttgtgacatgtcattctccgaataagggcaatgctgactaccttacgcgctggggaagacacatctcatggaaaaaggtcccgaaaggtcccgaaagggaagttcgatttctttcttgttcgaagtctctttccccttgctgacagcaagaagtgaggacatatcaagtccgtaaccaactccaacacctcgcgcccttcccactttgaccccctgagacattccattccgacagatctcatcatctcccccccccccccccgttttcactccctgctagctactctacaatcttgccgggatgaaagactcctccgccggcgcctcactcttctgcagcagcgtctccacccctttctggattcgcgccgccagcgctggactcaccagcttccaggtctcgtagacctgcttttggatctccgcgcgcgtctgcgcgacggactcgaccatattctggaccatggtgtcctggaactcctgcccgcggaagtccggcaggacctcgtagaagtcgcgggcgaacttgtagtcgtgctcggtggtctggagccaggacttggactggatctccttgagccactcgttgtacttggggtcgggctcgcggggctcggcgaagcggacgggctgctgctgggtcagaaagcccgggtgggcgccgtagttggcgtcgaaggtgccctggccgtcgcgcgcgatggggttgaaggtgtgggcggaccggttgacggggatctgctggtggttggcgcccagccggtaccgctgggcgtccgggtaggcgaagatgcgcgcctggaggatcgggtcctccgagggggccaccccgggcaccatgttcgagggggagtaggccagctgctcgatctcggcgaagtagttggccgggttgcgcttgagcgtcagcttgccgaagaccttgggcgggatcagcgtcacgtccttggggtacgtgcccaggttccagtgcttggtcacgtccaggatgttgtaccccaggttcggcgcgtccttggggtcgaccacctgcacgctcgccgtccacgtcggatactcgccgcgctcgatggcctcgtacaggtcgcgggtggcgtggtccggatccaggtcgttcgtgcccttggcctgctggccgtcggtgaagttgggcccgcgatcggaggagaggaagatgtggacgtatttgaacgacccgtcgggcttcacccatttgtacgcgtgggccatgtagccgctcatggaccgccagttgaacatggtcccgaagtcgctgaactgcagcaggaccatgtgcagcgactcgtggttgttggtcacccagtcccagtacatgttggggttcagcaggttggtctgcggatcccggcgctgggcgtgcatcaggctggggaacttgacggggtcgcggatgaagaagaaggggaagttgagcatcacccagtcccagttgccctcggtggtgtagaacttgcaggccatgcctttcaggtcgcgcattccctcgttggacccgcgttcgaggccagtcgtcgagaaccgcgtcacacagggggtcttcttgcccactcccaccagcatgtcgatatcgcagatatccgagatgtcggccgtcacctggaatggacggtccgggtcagcaactcagcttgggcgtggttccttccctcgtctgtaccaggatatgctgcctcatacctcaaattcgccataggctcccgctcctttagcatgcaccactcgctcggggatcttctcccggttgaagtgggcgagggtatcgaccagatggtggtcgctgagtgccagcgtccttccatccgccggaggaccctcaggcttgtggaagggacagccatttgccgtggtgtaggtcggcttgctatcgggcgccatgatttctgtgtgacaggtcttcgcttgatatcctcgctagatagcaagatgtaaacgggaacaagaaagaagacagggcaagtggtggtggaaatcaagtgcgtctggcgccgtagtcggtctccaccagacacagtaaatgtatttcaacacacatctggtcaccggtcgaggtagctgacgttgaccttgcagtccgcttcatcttcatcccttgcgtctgaagttgcctctccccaatttgttttccgaccctgtcgtcgtttcagatcccttgcatcttacacccaagcgagtccttgccgccgtccacgcccccactcattctcgctttgccacttgcaagggtcagatgcggtcatcaagctgcggaaggaagatgaagggctagcccctgctgtatgcgattgatgcatacaagcttgcctccaggggagacaaccaagggggggggaagggcagcgtagaccgcctctttgtcttcactcgcagagaccagtctctattccactttcaagactgtctgtgacaaggctaatcctacgagcctgtcatcccggttccgccctcgacagcattcggcctagacctcggaatataacatggctgccgggctggaaatggtgaacaccccgccaccgtgaccgtgaccgagaaaagaaaccctcaacatgggcagcatcaatcccccgattttggacatccgccagtcgaccttcgagacctccatcccccaacaagtaaccgagggactcaccaaagaccccaaaaccctgccggcccttcttttctacagcggcgatggcatccagcactggacgcgccactcgtgcgccccggatttctacccccgccgcgaggagatcaacatcctccacagcgaagcggctcacatggcggcctcgataagccaggacagtgttgtggtggatctgggcagcgcgtatgcaagcacacacaatctacaatcaaagcccagcagaggtgtcggggaaatgaataactaaaccgcaaacccaatgcaccttatagatccctcgataaagtcctccccttcctgcacgccctcgaggcccaacagaagcgcgttcactactacgccctcgacctgagctacccggtgctcgcctcgacgctgtccgagatccccgtggcgcagttccagtatgtccagatcgccgcgctgcacggcaccttcgaggacgggctgcagtggctgcacgactcgcccgaggtccgcgagcgcccccaccacctgctgctgttcggcctcacgatcggcaacttctcacggcccaacgccgccgcctttttacggaatatcgccgaccgggcgctggcggcgtcccccgcggagagctccatcctcctcaccctggacagctgcaagatgccgaccaaggtgctgcgcgcgtacacggcggaaggggtggtgcctttcgcgctgacctcgttgagctatgccaatcagctcttctggccgggtggggagggaagggtgtttgatgaagaggaatggtatttccacagtgagtggaacttcgcgctggggcgccacgaggcggcgctcatcccccgggatcgcgatatcaccctcgggccgccgttggagcaggtcgtcgtgcgccggggtgagaaggtgaggtttggctgcagctacaagtatagtgctgcggagcgggagcatttgttcgctggggcggggttggagaatgtggcttcctggtcggccaagggctgtgatgttgctttttatcagttgaagatgcggcccgagtgagggcttgacgtggtcatttccgggtgggattgaacagggttttggtgcagagtacaccttgttactaggaagtttcgtcaggaactgcagtttacccgttcgtatttaatctatgagagcacattaagcgggaaggagattggtattcgacaacaacaacattgtgtctttctttttgtatttggattggaacacatatccggtagccacactgcgctgacggccatcacaacaggaaaatgttgttgcacagtggtggttttcattaatctaccttagtaggaggtattgggacactcttgacatcgcttcgagaccaatattcattgaagatcaagcaacggagcagcttgaaatctcatgaacagatggtgattaagagaaaaaacaagcaaaagcaaaaacatacaacagaagggattcgctggtggtcacccacccaactactaacctcccggcgtgtggcttaagtacggctgagcggacgggaagccctgttttccacaccctgtggtcgtatgtgcttgttttaacagcactttaatttatatacaggttttcttcagatcagtactatgtttagaacacatacaaattgtatcaaggaaaagcgagaggaggaggaccagacttatgtgtggcagccgcaatcttagaaatctttctgatgaatgaggtctgaatatggtgaggctagggctacttgtgttctgataactacctaactacataatctccgagatcttccctgggtacccggcaggtacctaggtatattgttatgctgaacacgccaatagacacctgtacaagcaactggaggtgatgtaaacactcataccagtgggctcacccactcgctcgtatacgattaacaatcaggtcaatagaataagctatccacatacgccgacgcctcctttcccttgacaacccaccgttcccaggagagctcatctctagccatctcccactccggcaagacctcttcatccttctcaccggggtacctgaagcaatactcatagccctggccgtcagccctaagctgacgagcatactggccatcacgcagcccagccggacgatatcccggcacgtgcttcagtcgaaaccccaccgtgggatggatgcactcgcaagtatgcccgagaacctcattcgtgcggacatcccgaaaatagcgacgcggcctgcgcgccagcgacccgttcaggcggtaggtgaagtcgaagctgtcgggcaagtcgccggtggcccagccgtactgacggatctggcgataggccgggggagcatcgcccgacggggtgagcgggtgttgcacggcggtaatcagaccctgccctgtgtgccacagccactgggcccaggactcggactgccggcgacgttctttcttctggtacgcctccagcgcctcgttgatccgccgcagacgatcctctcgagtcttttcctcgagctggaggagcgtgcggtcgagggagacgtacccctggatctggtccagcatccacgcgaagacgatgtgggacatctcttccatgttgcccttgttcttgagggtctggttggatccgcccccgatgtgaatgtggaaccccggaaaccagacctgcgtcagctgggacggctccttctggctgtcctcccattccaaaagctcacgggcagccgcgttgcgtcgtttctcggctgcgtgtaactcctcggtgctggcggcaccgcttcttacgagttttagccactcgcgggtggcgttccgtgcctcctgcttgatcgctcgcgattggctctccgtgatattcttcggcgccgggacgaagaacatcgccgggcggaacagtcggcggtgctcgtccaacgccagcgcctggaatgcattcttgatgtctgggtttgggatcagtatcgtctcatcctgagccaatgatagcctggagggattgcttactaggcgaaagaccgacattgtgccattcaggcttatcagtccaggtcagccaggcagggagctcatggccgaacagctcggggaagcccagattgccgacggtatcgaagaccccgaccacttgcacctgccgtgactcgggggtcgatgcccattctgcggccgactcccaaaaaccctcctcttcttctgccccatcctcctcctcttccccctcttctccgccgaaggaggatcggcgcccccggaaaaactcatagtacgcatcactgccatagaaccgctccccgggcggcgtcttcttatacaccttccacagcgccgggaaattcgcgatctggtgcggcgggcagatgccgatgtccgagatgaggccggcgatggcgcgcgcggtgtaggcgccgcgcgagaacccaaagcacaggatctggtcgcccgggttccagttcagggcgcagaagtggtaggcctcgacgatgttgccctccagcccttggcccagggcgccctcggtgaagttgcgcagcgggcccgccgccgtggtgttggtgcccaccccggagtcgtaccagacgatctgctgccagggctggtggtcgtgcgggtcgatccccacgctgtcgatggcgcggcagagccgcgtcacgttcgaggggatgcgctcttggccgtcgacggcggattgccaggtgccgtcgcagcagatgaggaggcgcttgcgcgcgggttggggcttgggggtgggggtgagggggcggtgcatggtgttctctctttgaatggttaagagcgaagattaatagattatttgagatctccaaaggagttatctaccttctctttcctctagaagggtcgaggggaatcagctgggcttatcattcatgtactgtacattcatgtggtgtccgtccgtcgctacaaattgcctgctcggcagaaatgaccatcttttgttctccgtgtcaggagctgatgacccttttgcatctcagctgaactgttctttgcgagttgattggtagtcatttccttcgagtcttcctcttcagctccggctggataccccaagcgttcgtggcgcattttcagctcaaaccacgtccgtgtcattggagccctaattccaccgcaccccgcacccccggatattggaaagagctcaagaacaacactatttcttagaaaattggggaccagcaaatttgtcgttctccatattccccactcccctttgttcgtggctatataaggctgacaaacatgattgtcgtacaagttcataacaacctactagcatcaagcacgatatatctcagtattcttccatcacttccgcagtaaccatgtcagtcaaacagcatcccatacccaccagacgagagccatagagtatacgcaaggccaattctcactgcaagcacgaagcctcagaagacgaacgaaagaacccaccaacccaacccaccagaaccccaa

SEQ ID NO: 31 - DNA, 17501 bp, Aspergillus japonicus SAITO IFO 4060
  misc_feature (1)..(1688): orf11
cgttaactgatattgaaggagcattttttgggcttggctggagctagtggaggtcaacaatgaatgcctattttggtttagtcgtccaggcggtgagcacaaaatttgtgtcgtttgacaagatggttcatttaggcaactggtcagatcagccccacttgtagcagtagcggcggcgctcgaagtgtgactcttattagcagacaggaacgaggacattattatcatctgctgcttggtgcacgataacttggtgcgtttgtcaagcaaggtaagtggacgacccggtcataccttcttaagttcgcccttcctccctttatttcagattcaatctgacttacctattctacctaagcattcatgaccatcctcgtcctcggcggccgcggcaagaccgccagccgcctctcagccctcctgcacgaagcccagatccccttcgtcgtgggcacctcctccacggccccgatcgccacctaccgaaccgcccacttcgactggctgaacaaagacacctggaccaacgcgtgggaccaggccccggagcccatcaccgctatctacctcgtaggcgcccacgccccggagttcgtgcacccgatgttcgagttcatcgacctggcgcacagccggggcgtgcggcggtacgcgctcgtcagcgcgagcaacaccgaaaagggggatccgatgatgggggaggcgcacgcgcacctggactcgaaagagggggtcaagtatgtggtgttgcggccgacgtggtttatgggtgcgttgtctggcttttcccttgccgtttcctcgcgtttccctctgagaaggggattggcgtgttttcctatctcgaatactttccccctgcccctaacccccaaatcccaaagtcaggactaattatggagtgaaacagaaaaccacatcgaagacccccacctcagctggatcaagacggagaacaagatctactcggccacgggcccgggcaagatccccttcgtctcggcggatgatatcgcccgcgtcgccttccacgccctgacgcagccgctcacccagaaggagcagcagtacatggttctgggcccggatctgctgtcctatgctgatgtttgtttcttcccaccttccaccccccacctcccatctttccctatctcctaccacggctatccgattcccagataagggtatgctcatgacgagaatgcgatgatactaatgctggagggaaaacagatggccgagatcctcaccgacctcctgggccggaagattacgcacgtcaccctgcccgccgagaagttcggccagttcctggtcgacacggccgggttgcccccggactttgcggagatgctggcgacgatggaggcggcggtggcgacgggggcggaggagcgggtcagccaagatgtcgagaaggtgaccgggcggccgccgcggcggtttgtggattacgcgagggagaatcgcgagcattggttgtgatttcgctggtggggtttgttcgggatgatgtttggtgggagttgggaattggcacttggtctgcagtgcaaaggttcactcagtactcagtatatcctagagtctcaaagtgttgaagggcgggctctggagcctgcggtgaataaaaccgagacttggtgccatacgcttgctaggtcatcgatctcgattgagataggcgttaactgatattgaaggagcattttttgggcttggctggagctagtggaggtcaacaatgaatgcctattttggtttagtcgtccaggcggtgagcacaaaatttgtgtcgtttgacaagatggttcatttaggcaactggtcagatcagccccacttgtagcagtagcggcggcgctcgaagtgtgactcttattagcagacaggaacgaggacattattatcatctgctgcttggtgcacgataacttggtgcgtttgtcaagcaaggtaagtggacgacccggtcataccttcttaagttcgcccttcctccctttatttcagattcaatctgacttacctattctacctaagcattcatgacgatcgacccctcctcccccggggcgcccgcctcgaagctcttccaacccatgaagataggcctatgtcacctccagcaccgcctcatcatggcccccaccacccgctaccgcgccgacagcaccgcgacgcccctccccttcgtgcaagaatactacgcccaacgcgccgccgttccggggacactcctgatcaccgaagccaccgacatctccccgcaggcggccggagaaccgcacatcccaggcctgtacagcgcaacccagatcgccgcgtggcgggggatcgtctccgccgtgcaccgccggggctgctacatcttctgccagctgtgggcgaccggacgggcggcggaccccgccctgttgaaggagaaggggttccctctcgtctcgagcagcgatgtgcccatcgagactgaatcgcagcagccgcagccgctgacgcaggccgagatcgaggggtacatcgcggacttcgtgacggcggcaagggtagcggtccacgaggtggggttcgatggggtggagctgcacggcgccaacggatacctcatcgatcagttcacgcagtggccgtgcaaccagcggacggattgctggggcgggagcatcgagaaccgcgcgcggtttgcgctggcggtcactcgcgcggtggtggatgccgtcggggcggagcgcgtcggggttaagctgtcgccctggagtcagtacgccgggatgggggtgatggctgatttggtgccgcagtttgagttcttgatcaaaaggttacgggaggagtacaatgatggtcctgggctggcgtacttgcatctggcgaattcaaggtggttggatgaggagacttcccatccggatccgcatcatgaggtcttcgtgcgcgcgtggagggggtcgggcagggatggtattgaggcgaagaaagaggcgcctccggtgatcctggccggcggctatgatgctgtctcggcgcccgaggtcgctgataaggtgttcaaggaagaggacaatgtcgccgtcgcgtttgggcgatattttatctcgacgcccgacctgccgttccgcatgcaacggggcattccattgcagaagtatgatcgcgcctcgttctatacctcgcttttgaaagaaggttatctggattatccgtacagtgcggagtatgttgcggagtttgggcagaggctgaagaagaaggaggcggcggcgacagggtcgatatgtcagggttgaatggatagggtgggatcgtctgcgtatgttgcgagagcctggcgtgaaaccttttaagcaaagtagattatagaaatagatcggctacgtagggaggcccactgtgaaaaatcatgtaacgcgagcttaatgatagagagaagcacaaaacgttaactgatattgaaggagcattttttgggcttggctggagctagtggaggtcaacaatgaatgcctattttggtttagtcgtccaggcggtgagcacaaaatttgtgtcgtttgacaagatggttcatttaggcaactggtcagatcagccccacttgtagcagtagcggcggcgctcgaagtgtgactcttattagcagacaggaacgaggacattattatcatctgctgcttggtgcacgataacttggtgcgtttgtcaagcaaggtaagtggacgacccggtcataccttcttaagttcgcccttcctccctttatttcagattcaatctgacttacctattctacctaagcattcatgaccgtcgtcaactcgcgcatctttgcagtcaccggcggcgcctccggcattggcgcggccacctgccgcctcctcgccgcgcgcggcgcagcggtcatctgcatcggggacatctcgcccaagggcttcgagagcgtcaccagcgacatccaggccctcaacccggccaccaaggtgcactgcacccggctggacgtctcctcgtccgccgaggttgacgcctggctacagaccatcgtgcagaccttcggggacttgcacgggctggccaacgtcgcgggcatcgcccagccggcgggcgcccgccaggccccggccctgctcgaggagacggacgaggaatggcagcgcgtgttcaaggtcaacctggacggggtcttctactgcacgcgcgcaggtatccgcgcgatgaaggcccttccggggcccaaggaccgcgccatcgtcaacatcggtagcatcgccgcgtttattcacttgccggatgcctacgcctacgggacctccaagggcgcctgcgcgtacttcaccagctgtgcggccatggatgcctggcccctggggattcggatcaacagtgtctcgccgggtaagtgtctttcgtgctgtttattgtatagatctatggacagaggagaggaggatgaccctttggcgaggggggaaagctaacattgaggaaacaggtatcaccgacaccccggctctgccgcagttcatgccccaggcgaagtccatcgatgaggtcaaggaagcgtacatcaaggaggggttccctatcattcagcctgaggatgtggccaggacgattgtctggctgctcagcgaggattcccgcccggtgtatggggccaatatcaatgttggggctgctactccttgagatgctagatgctgtttagctagcttgagaggaggtgtttatatctagagacttggtgttctccatcttaatgacagccaaggttgagcttcaagtcggattcgaaatgtagtagggttggtttatctcgactgtatctacgttaactgatattgaaggagcattttttgggcttggctggagctagtggaggtcaacaatgaatgcctattttggtttagtcgtccaggcggtgagcacaaaatttgtgtcgtttgacaagatggttcatttaggcaactggtcagatcagccccacttgtagcagtagcggcggcgctcgaagtgtgactcttattagcagacaggaacgaggacattattatcatctgctgcttggtgcacgataacttggtgcgtttgtcaagcaaggtaagtggacgacccggtcataccttcttaagttcgcccttcctccctttatttcagattcaatctgacttacctattctacctaagcattcatgaccatcaccgagacctccaagccgacgctgcgccgcatcccccggagcgccggcgacgaggccatcttccaagtcctgcaggaagacggagtcgtcgtgatcgaaggattcatgagcgcggaccaggtccgccgcttcaacggggagatcgacccgcacatgaagcagtgggaactgggccagaagtcgtaccaggagagctacctggcggggatgcgacagctgagcagcctgccgctgttcagcaagctcttccgggacgagctgatgaacgacgagctcctgcacgggctgtgcaagcggctgttcgggcccgagtccggcgactactggctgacgacctcctcggtgctggagacagagccgggctaccacgggcaggagctgcaccgcgagcacgacgggatccccatctgcaccaccctgggccgccagagccccgagtccatgctcaacttcctcaccgcgctgaccgacttcaccgcggagaacggcgccacccgcgtcctgccgggcagtcacctctgggaggacttcagcgccccgccccccaaggccgacaccgccatccccgccgtcatgaaccccggcgatgccgttctcttcaccggcaagaccctgcacggcgccggcaagaacaacaccaccgacttcctgcgccgcggcttccccctgatcatgcagtcgtgccagttcacccctgtcgaggcctcggtggccctgccccgtgagctggtcgagaccatgacccccctggcccagaagatggtcggctggcggaccgtctccgctaagggggtggatatctggacgtatgatctgaaggatctggcgacaggaattgatctcaagtcgaaccaggtggcgaaaaaggcgtgagtgggagacttgaccgcaaaataggaaccagtccgttgtcttaggtcagccaaggaaatgtgaaaggaaatctatatcataaatttgttccaaccatttgttccagactgtagacaagaaacaagattgttcgcgagtatgccgtgtgaaccaacatagtacaacttataccctgcctgctacagccttagcatgccattcgttaactgatattgaaggagcattttttgggcttggctggagctagtggaggtcaacaatgaatgcctattttggtttagtcgtccaggcggtgagcacaaaatttgtgtcgtttgacaagatggttcatttaggcaactggtcagatcagccccacttgtagcagtagcggcggcgctcgaagtgtgactcttattagcagacaggaacgaggacattattatcatctgctgcttggtgcacgataacttggtgcgtttgtcaagcaaggtaagtggacgacccggtcataccttcttaagttcgcccttcctccctttatttcagattcaatctgacttacctattctacctaagcattcatgactgctgggcaaggcatcaagacgggcaatgcctctgactgtgaagtttaccgcacgctgagcgtcgccctcgactttgccaaccaggatgaggagctctggtggcacagcaccgcgcccatgtttgctcagatgctgcagtccaccaactacaacctccatgctcagtacaagcacctgctcatctacaagaaaaacgtcatccctttcctcggggtttatcccacaaatgacaagccccggtggctaagcatcctgacccgatacggcaccccctttgagctgagcctgaactgctccggcccactggtgcgctacacctacgagcccatcaacgcggccacggggacggcgcgcgacccgttcaacacccacgctgtctgggacagtctggaacagctgatggcgctgcagagcgggattgacctggacctcttccgccacttcaagaacgacctcaccctcagcgctgaggaatctgagtacctgtacaagaacaacctggtgggcgagcagatccggactcagaacaaactggcgctggatctccaggacggcgagttcgtggtcaagacctacatctacccggccctgaagtcgctcgccacgggcaggtccatccacgagctggtcttcgggtctgccttccgctggtccaagcagtacccggagctccgcaagcccctggacacactggagcagtacgtctactcgcggggccccagcagcacggccagcccgcgtctcctctcctgcgacctgatcgaccccaccaagtcccgcatcaagatctacctgctggagcggatggtcaccctggaggccctggaggacctctggaccatggggggcgagcgcaccgacgcctcgaccctggcggggctcgagatgatccgcgagctgtgggagctgatccgcctgcccgcgggcttgcagtcgtatccggccccgtatctgcccatcgggaccatcccggacgagcagctgcccctgatggccaactacaccatccaccacgacgaccccgttccggagccgcaggtctacttcaccaccttcggccgcaacgacatgcagatcgccgacgcgctggccaccttcttcgagcgccgcggctggcacgagatggcgcgccagtacaaggccgagctgtgcagccactacccgcacgccgaccacgagaccctgaactacctgcacgcctacatctccttctcctaccgcaagaacaaaccctacctcagcgtgtatctgcagtcgctggagacgggcgactgggtcacttgtaagctctccccttccgtgttcctttcctgcacacttccccaccgtggatacatccctgatgatctgacctgttcgcgcagcatccttcaactctgtccatgtggaccccggcctctcagccaccgtccaagagctgtccaagctgaccaagaccgccgggaccaccgttcgcgagacgaaactcccactcaccccggatggatcggagcccggcgtcatcacccagtactaggaaatcatctcgttggaccggatttgccagcttcccgccggggagttgcaagtgctttgtatgtaggtaggggaactagatgaaaaagagcaatcgaccggcgagggtcacacgcccggctctgcatgccgtttgccagtgattgtatgtagtaatgatagcctcatgagacagccatttagcttgggcttaatccagttggagtagaaccattgaagttaagttacttaaattcacctccccagtgcttgctggtgtagactttgaccttcgggtgcatggtgtaggacttggctagatgtctccgtgaaatgaacaatctttgactttaacgttaactgatattgaaggagcattttttgggcttggctggagctagtggaggtcaacaatgaatgcctattttggtttagtcgtccaggcggtgagcacaaaatttgtgtcgtttgacaagatggttcatttaggcaactggtcagatcagccccacttgtagcagtagcggcggcgctcgaagtgtgactcttattagcagacaggaacgaggacattattatcatctgctgcttggtgcacgataacttggtgcgtttgtcaagcaaggtaagtggacgacccggtcataccttcttaagttcgcccttcctccctttatttcagattcaatctgacttacctattctacctaagcattcatgggtcagtcacgagggatcttgggaggcgtgaggcaacttatcctggttattctggttggggcatacctcagccggctttcagcggtggatgacgaccgccacgactgtcgatgtcggccgggggagccatgctggcccacggtcagccactggagtactctgaacgagtccatcggcggtgctctggctcatgtcaaacccatcgctcacgtctgtcaccagtctggtcaggactcgtcggcttgcgagcaggtcttgcaagagagtcttgacagcaagtggcgggcgtcgcacacgggttagctcattccacctccctgaattcatctgacacacgggctcatccatcccacacaggcgcattgcaagactgggtctgggaaggtggcgcggaatccaaccagacatgttactacctgcggtccggtccggctggatcatgccaccagggccggattcccctctactccgcggctgtcaagtcggccagcgacgtgcagaaggtcgtggacttcactcgtcaacacaatttacgattagtgatccgcaacaccggccacgatgggagcggccgatccagtggaccggactctgtcgagatccacacccaccatctgaacagcgtgcaatatcaccccaacttccgccccgctggatcttctgagcgccagtcggctccaggacagccagcggtgacagtaggcgccggaatcctgcttggggacctctacgcccgcggcgcctcggagggatggatcgtagtgggcggggagtgtcccaccgtcggggcggccggcggattcctccagggcggaggagtatcgagttggctgagctacgcgcatggattggcagtggataatgtgcttgagtatgaagttgtgacggccaaggtgcgtgcgggttcatagcttccctccctccgcgctctaaccctaactaaccctccgcaaatgtccagggagagattgtcatcgccaacgcccatcaaaaccccgatctcttctgggcactgcgcgggggaggaggaggcacctttggcgtcgtgacccaggccacgcttcaagtccaccccgacctccccgtcagcgtcgccgacgccgtggtgacgggctcccgcgccgacgcgaccttctggtcccacggcgtcgccgccctcctgcgagcgctacaattcctcaacaaccacggcacggcaggccagttcatcctccgcaacaccgacgacgacacggtgcaagccagtctgacgatgtacttctcgaacctgaccgtccccgccgtcgccgacgagcgcatggacccgctccgccgcgccctcgaacacaacggacacccctaccagctcacctcgcggttcctgccccagatcagcagcaccttccggcacaccgcggatcgctacccggaggactacggaatcctgatgggctcggtcctggtgtcgctggacctgttcaattccgcgacgggccccgcggcgctggcgcagcacttcgcgcggctcccgatgacccccgacgatctcctgttcaccagcaacctgggcgggcgagtgtcctccctcaaccgcgacccggccagcacagccatgcatcccggctggcgtgacgccgcgcagttgctcaacttcgtccgcggggtcggggccccttctctcgcggctaaggccaccgccctccacgagctgcagacggtccagatggcgaggctgtatgagattgaaccggccttccagatctcgtaccgcaacctgggagatccctcggagcgccgcagtcgggaggtctattgggggtcgaactacgcgcggttagtggaggtcaagcggaggtgggatccggagggactgttcttcagcaagctggggattgatggtgatgcatgggacgcggaaggaatgtgtcgtcagacgcgccaaggggcgtggaatgtggcggtcaaatgggtccagagcttccttgggtctttgtctgcttctgtggtgtagttcattctctcttgatcgattggggaaatgcagttcgacatgataatattgttgtcgggatctcatgtaggataggtcgcctgcgattggagcggtcagattgcaagtgaccgtacgtgcagctgctgacctgccctgccacatttctaagcttggaaggggcagcaagacaggggggttgcactcggggggggctactccttgctggcggcgggtacattcttcgccagagcatgcaggataatgtggccgttaactgatattgaaggagcattttttgggcttggctggagctagtggaggtcaacaatgaatgcctattttggtttagtcgtccaggcggtgagcacaaaatttgtgtcgtttgacaagatggttcatttaggcaactggtcagatcagccccacttgtagcagtagcggcggcgctcgaagtgtgactcttattagcagacaggaacgaggacattattatcatctgctgcttggtgcacgataacttggtgcgtttgtcaagcaaggtaagtggacgacccggtcataccttcttaagttcgcccttcctccctttatttcagattcaatctgacttacctattctacctaagcattcatggcgcccgatagcaagccgacctacaccacggcaaatggctgtcccttccacaagcctgagggtcctccggcggatggaaggacgctggcactcagcgaccaccatctggtcgataccctcgcccacttcaaccgggagaagatccccgagcgagtggtgcatgctaaaggagcgggagcctatggcgaatttgaggtatgaggcagcatatcctggtacagacgagggaaggaaccacgcccaagctgagttgctgacccggaccgtccattccaggtgacggccgacatctcggatatctgcgatatcgacatgctggtgggagtgggcaagaagaccccctgtgtgacgcggttctcgacgactggcctcgaacgcgggtccaacgagggaatgcgcgacctgaaaggcatggcctgcaagttctacaccaccgagggcaactgggactgggtgatgctcaacttccccttcttcttcatccgcgaccccgtcaagttccccagcctgatgcacgcccagcgccgggatccgcagaccaacctgctgaaccccaacatgtactgggactgggtgaccaacaaccacgagtcgctgcacatggtcctgctgcagttcagcgacttcgggaccatgttcaactggcggtccatgagcggctacatggcccacgcgtacaaatgggtgaagcccgacgggtcgttcaaatacgtccacatcttcctctcctccgatcgcgggcccaacttcaccgacggccagcaggccaagggcacgaacgacctggatccggaccacgccacccgcgacctgtacgaggccatcgagcgcggcgagtatccgacgtggacggcgagcgtgcaggtggtcgaccccaaggacgcgccgaacctggggtacaacatcctggacgtgaccaagcactggaacctgggcacgtaccccaaggacgtgacgctgatcccgcccaaggtcttcggcaagctgacgctcaagcgcaacccggccaactacttcgccgagatcgagcagctggcctactccccctcgaacatggtgcccggggtggccccctcggaggacccgatcctccaggcgcgcatcttcgcctacccggacgcccagcggtaccggctgggcgccaaccaccagcagatccccgtcaaccggtccgcccacaccttcaaccccatcgcgcgcgacggccagggcaccttcgacgccaactacggcgcccacccgggctttctgacccagcagcagcccgtccgcttcgccgagccccgcgagcccgaccccaagtacaacgagtggctcaaggagatccagtccaagtcctggctccagaccaccgagcacgactacaagttcgcccgcgacttctacgaggtcctgccggacttccgcgggcaggagttccaggacaccatggtccagaatatggtcgagtccgtcgcgcagacgcgcgcggagatccaaaagcaggtctacgagacctggaagctggtgagtccagcgctggcggcgcgaatccagaaaggggtggagacgctgctgcagaagagtgaggcgccggcggaggagtctttcatcccggcaagattgtagagtagctagcagggagtgaaaacgggggggggggggggagatgatgagatctgtcggaatggaatgtctcagggggtcaaagtgggaagggcgcgaggtgttggagttggttacggacttgatatgtcctcacttcttgctgtcagcaaggggaaagagacttcgaacaagaaagaaatcgaacttccctttcgggacctttcgggacctttttccatgagatgtgtcttccccagcgcgtaaggtagtccgttaactgatattgaaggagcattttttgggcttggctggagctagtggaggtcaacaatgaatgcctattttggtttagtcgtccaggcggtgagcacaaaatttgtgtcgtttgacaagatggttcatttaggcaactggtcagatcagccccacttgtagcagtagcggcggcgctcgaagtgtgactcttattagcagacaggaacgaggacattattatcatctgctgcttggtgcacgataacttggtgcgtttgtcaagcaaggtaagtggacgacccggtcataccttcttaagttcgcccttcctccctttatttcagattcaatctgacttacctattctacctaagcattcatgggcagcatcaatcccccgattttggacatccgccagtcgaccttcgagacctccatcccccaacaagtaaccgagggactcaccaaagaccccaaaaccctgccggcccttcttttctacagcggcgatggcatccagcactggacgcgccactcgtgcgccccggatttctacccccgccgcgaggagatcaacatcctccacagcgaagcggctcacatggcgtatgcaaggcctcgataagccaggacagtgttgtggtggatctgggcagcgcgtatgcaagcacacacaatctacaatcaaagcccagcagaggtgtcggggaaatgaataactaaaccgcaaacccaatgcaccttatagatccctcgataaagtcctccccttcctgcacgccctcgaggcccaacagaagcgcgttcactactacgccctcgacctgagctacccggtgctcgcctcgacgctgtccgagatccccgtggcgcagttccagtatgtccagatcgccgcgctgcacggcaccttcgaggacgggctgcagtggctgcacgactcgcccgaggtccgcgagcgcccccaccacctgctgctgttcggcctcacgatcggcaacttctcacggcccaacgccgccgcctttttacggaatatcgccgaccgggcgctggcggcgtcccccgcggagagctccatcctcctcaccctggacagctgcaagatgccgaccaaggtgctgcgcgcgtacacggcggaaggggtggtgcctttcgcgctgacctcgttgagctatgccaatcagctcttctggccgggtggggagggaagggtgtttgatgaagaggaatggtatttccacagtgagtggaacttcgcgctggggcgccacgaggcggcgctcatcccccgggatcgcgatatcaccctcgggccgccgttggagcaggtcgtcgtgcgccggggtgagaaggtgaggtttggctgcagctacaagtatagtgctgcggagcgggagcatttgttcgctggggcggggttggagaatgtggcttcctggtcggccaagggctgtgatgttgctttttatcagttgaagatgcggcccgagtgagggcttgacgtggtcatttccgggtgggattgaacagggttttggtgcagagtacaccttgttactaggaagtttcgtcaggaactgcagtttacccgttcgtatttaatctatgagagcacattaagcgggaaggagattggtattcgacaacaacaacattgtgtctttctttttgtatttggattggaacacatatccggtagccacactgcgctgacggccatcacaacaggaaaatgttgttgcacgttaactgatattgaaggagcattttttgggcttggctggagctagtggaggtcaacaatgaatgcctattttggtttagtcgtccaggcggtgagcacaaaatttgtgtcgtttgacaagatggttcatttaggcaactggtcagatcagccccacttgtagcagtagcggcggcgctcgaagtgtgactcttattagcagacaggaacgaggacattattatcatctgctgcttggtgcacgataacttggtgcgtttgtcaagcaaggtaagtggacgacccggtcataccttcttaagttcgcccttcctccctttatttcagattcaatctgacttacctattctacctaagcattcatgcaccgccccctcacccccacccccaagccccaacccgcgcgcaagcgcctcctcatctgctgcgacggcacctggcaatccgccgtcgacggccaagagcgcatcccctcgaacgtgacgcggctctgccgcgccatcgacagcgtggggatcgacccgcacgaccaccagccctggcagcagatcgtctggtacgactccggggtgggcaccaacaccacggcggcgggcccgctgcgcaacttcaccgagggcgccctgggccaagggctggagggcaacatcgtcgaggcctaccacttctgcgccctgaactggaacccgggcgaccagatcctgtgctttgggttctcgcgcggcgcctacaccgcgcgcgccatcgccggcctcatctcggacatcggcatctgcccgccgcaccagatcgcgaatttcccggcgctgtggaaggtgtataagaagacgccgcccggggagcggttctatggcagtgatgcgtactatgagtttttccgggggcgccgatcctccttcggcggagaagagggggaagaggaggaggatggggcagaagaagaggagggtttttgggagtcggccgcagaatgggcatcgacccccgagtcacggcaggtgcaagtggtcggggtcttcgataccgtcggcaatctgggcttccccgagctgttcggccatgagctccctgcctggctgacctggactgataagcctgaatggcacaatgtcggtctttcgcctagtaagcaatccctccaggctatcattggctcaggatgagacgatactgatcccaaacccagacatcaagaatgcattccaggcgctggcgttggacgagcaccgccgactgttccgcccggcgatgttcttcgtcccggcgccgaagaatatcacggagagccaatcgcgagcgatcaagcaggaggcacggaacgccacccgcgagtggctaaaactcgtaagaagcggtgccgccagcaccgaggagttacacgcagccgagaaacgacgcaacgcggctgcccgtgagcttttggaatgggaggacagccagaaggagccgtcccagctgacgcaggtctggtttccggggttccacattcacatcgggggcggatccaaccagaccctcaagaacaagggcaacatggaagagatgtcccacatcgtcttcgcgtggatgctggaccagatccaggggtacgtctccctcgaccgcacgctcctccagctcgaggaaaagactcgagaggatcgtctgcggcggatcaacgaggcgctggaggcgtaccagaagaaagaacgtcgccggcagtccgagtcctgggcccagtggctgtggcacacagggcagggtctgattaccgccgtgcaacacccgctcaccccgtcgggcgatgctcccccggcctatcgccagatccgtcagtacggctgggccaccggcgacttgcccgacagcttcgacttcacctaccgcctgaacgggtcgctggcgcgcaggccgcgtcgctattttcgggatgtccgcacgaatgaggttctcgggcatacttgcgagtgcatccatcccacggtggggtttcgactgaagcacgtgccgggatatcgtccggctgggctgcgtgatggccagtatgctcgtcagcttagggctgacggccagggctatgagtattgcttcaggtaccccggtgagaaggatgaagaggtcttgccggagtgggagatggctagagatgagctctcctgggaacggtgggttgtcaagggaaaggaggcgtcggcgtatgtggatagcttattctattgacctgattgttaatcgtatacgagcgagtgggtgagcccactggtatgagtgtttacatcacctccagttgcttgtacaggtgtctattggcgtgttcagcataacaatatacctaggtacctgccgggtacccagggaagatctcggagattatgtagttaggtagttatcagaacacaagtagccctagcctcaccatattcagacctcattcatcagaaagatttctaagattgcggctgccacacat

SEQ ID NO: 32 - DNA, 363 bp, Aspergillus nidulans
cgttaactgatattgaaggagcattttttgggcttggctggagctagtggaggtcaacaatgaatgcctattttggtttagtcgtccaggcggtgagcacaaaatttgtgtcgtttgacaagatggttcatttaggcaactggtcagatcagccccacttgtagcagtagcggcggcgctcgaagtgtgactcttattagcagacaggaacgaggacattattatcatctgctgcttggtgcacgataacttggtgcgtttgtcaagcaaggtaagtggacgacccggtcataccttcttaagttcgcccttcctccctttatttcagattcaatctgacttacctattctacctaagcattc

SEQ ID NO: 33 - DNA, 10058 bp, Aspergillus japonicus SAITO IFO 4060
ctagtgcaccgcttccacctattctacctgcttgacctatctaacctgtttaatctatttaatctattttatttgttgttttttttaagcttatctacatgatgagtccacaaggtatggagatgtagggctatcattctgccgaagggctgtgcggatgaaccatggtccgggtaaaggtttcggcgcagttcatccacgacctgctcacggcgggcatgttgagatgtctctgaacgtcgtacgtaagtttatcataccctgggaggcatggtgggcgtgtggtatctctaatgactggcaatggggaatccgccgcccgttcccaccactgataataccccgttgcggggtatctgcgcacatagctacgaattctcctctacggtacagtggcctatctcaatcgagatcgatgacctagcaagcgtatggcaccaagtctcggttttattcaccgcaggctccagagcccgcccttcaacactttgagactctaggatatactgagtactgagtgaacctttgcactgcagaccaagtgccaattcccaactcccaccaaacatcatcccgaacaaaccccaccagcgaaatcacaaccaatgctcgcgattctccctcgcgtaatccacaaaccgccgcggcggccgcccggtcaccttctcgacatcttggctgacccgctcctccgcccccgtcgccaccgccgcctccatcgtcgccagcatctccgcaaagtccgggggcaacccggccgtgtcgaccaggaactggccgaacttctcggcgggcagggtgacgtgcgtaatcttccggcccaggaggtcggtgaggatctcggccatctgttttccctccagcattagtatcatcgcattctcgtcatgagcatacccttatctgggaatcggatagccgtggtaggagatagggaaagatgggaggtggggggtggaaggtgggaagaaacaaacatcagcataggacagcagatccgggcccagaaccatgtactgctgctccttctgggtgagcggctgcgtcagggcgtggaaggcgacgcgggcgatatcatccgccgagacgaaggggatcttgcccgggcccgtggccgagtagatcttgttctccgtcttgatccagctgaggtgggggtcttcgatgtggttttctgtttcactccataattagtcctgactttgggatttgggggttaggggcagggggaaagtattcgagataggaaaacacgccaatccccttctcagagggaaacgcgaggaaacggcaagggaaaagccagacaacgcacccataaaccacgtcggccgcaacaccacatacttgaccccctctttcgagtccaggtgcgcgtgcgcctcccccatcatcggatcccccttttcggtgttgctcgcgctgacgagcgcgtaccgccgcacgccccggctgtgcgccaggtcgatgaactcgaacatcgggtgcacgaactccggggcgtgggcgcctacgaggtagatagcggtgatgggctccggggcctggtcccacgcgttggtccaggtgtctttgttcagccagtcgaagtgggcggttcggtaggtggcgatcggggccgtggaggaggtgcccacgacgaaggggatctgggcttcgtgcaggagggctgagaggcggctggcggtcttgccgcggccgccgaggacgaggatggtcattttgtcggagggggttaagaagaaggatagctgtcgagggcgaggaggtaagagccaaggtaggtttgggaggtttatttgctggcgataaggatttataccggatgtatggaaatggtgggccggagaatgagggggaggggagggggcggggtgcggtgaggtgatggcacaattgacaagcacactcaccattcaccattgacatgcaggatcgagactggagcaaatcgtgttcgatgaagattggtctgatgatttcccaactcaatgcgggagaaccttgctttggggacatttctggtgggttgcaagctacgttggtgctgcagctcgaagctcggatgcgaggatgagctgggcagaagggtggggcatgcaggatgcaggatgcatgcatttacggatgtagcgtttcagaattgggccactgcggcaatagggccacgtagtcctttattggtgtacggcggcctaaagaatgaggttgtgagaggcagaggcaagaagaaagtctgcgagaagcgaggagagagccgagttagggcttgggaggggggaggtgcagagcagagggagagagagtgtgcggccacggcggtcaatttaccgccacaaccacaagaaccaaacagtcggctggataaatatgtacaagcgatatcagtccggtcaatggcactaaaatagcccaaccttcggctcttgtccagcttcaattgtatgctgcaatagcgccccgccacccaacaacatgacgatcgacccctcctcccccggggcgcccgcctcgaagctcttccaacccatgaagataggcctatgtcacctccagcaccgcctcatcatggcccccaccacccgctaccgcgccgacagcaccgcgacgcccctccccttcgtgcaagaatactacgcccaacgcgccgccgttccggggacactcctgatcaccgaagccaccgacatctccccgcaggcggccggagaaccgcacatcccaggcctgtacagcgcaacccagatcgccgcgtggcgggggatcgtctccgccgtgcaccgccggggctgctacatcttctgccagctgtgggcgaccggacgggcggcggaccccgccctgttgaaggagaaggggttccctctcgtctcgagcagcgatgtgcccatcgagactgaatcgcagcagccgcagccgctgacgcaggccgagatcgaggggtacatcgcggacttcgtgacggcggcaagggtagcggtccacgaggtggggttcgatggggtggagctgcacggcgccaacggatacctcatcgatcagttcacgcagtggccgtgcaaccagcggacggattgctggggcgggagcatcgagaaccgcgcgcggtttgcgctggcggtcactcgcgcggtggtggatgccgtcggggcggagcgcgtcggggttaagctgtcgccctggagtcagtacgccgggatgggggtgatggctgatttggtgccgcagtttgagttcttgatcaaaaggttacgggaggagtacaatgatggtcctgggctggcgtacttgcatctggcgaattcaaggtggttggatgaggagacttcccatccggatccgcatcatgaggtcttcgtgcgcgcgtggagggggtcgggcagggatggtattgaggcgaagaaagaggcgcctccggtgatcctggccggcggctatgatgctgtctcggcgcccgaggtcgctgataaggtgttcaaggaagaggacaatgtcgccgtcgcgtttgggcgatattttatctcgacgcccgacctgccgttccgcatgcaacggggcattccattgcagaagtatgatcgcgcctcgttctatacctcgcttttgaaagaaggttatctggattatccgtacagtgcggagtatgttgcggagtttgggcagaggctgaagaagaaggaggcggcggcgacagggtcgatatgtcagggttgaatggatagggtgggatcgtctgcgtatgttgcgagagcctggcgtgaaaccttttaagcaaagtagattatagaaatagatcggctacgtagggaggcccactgtgaaaaatcatgtaacgcgagcttaatgatagagagaagcacaaaacatcgtctcccagggtgatcagccatgggtgatagatacagtcgagataaaccaaccctactacatttcgaatccgacttgaagctcaaccttggctgtcattaagatggagaacaccaagtctctagatataaacacctcctctcaagctagctaaacagcatctagcatctcaaggagtagcagccccaacattgatattggccccatacaccgggcgggaatcctcgctgagcagccagacaatcgtcctggccacatcctcaggctgaatgatagggaacccctccttgatgtacgcttccttgacctcatcgatggacttcgcctggggcatgaactgcggcagagccggggtgtcggtgatacctgtttcctcaatgttagctttcccccctcgccaaagggtcatcctcctctcctctgtccatagatctatacaataaacagcacgaaagacacttacccggcgagacactgttgatccgaatccccaggggccaggcatccatggccgcacagctggtgaagtacgcgcaggcgcccttggaggtcccgtaggcgtaggcatccggcaagtgaataaacgcggcgatgctaccgatgttgacgatggcgcggtccttgggccccggaagggccttcatcgcgcggatacctgcgcgcgtgcagtagaagaccccgtccaggttgaccttgaacacgcgctgccattcctcgtccgtctcctcgagcagggccggggcctggcgggcgcccgccggctgggcgatgcccgcgacgttggccagcccgtgcaagtccccgaaggtctgcacgatggtctgtagccaggcgtcaacctcggcggacgaggagacgtccagccgggtgcagtgcaccttggtggccgggttgagggcctggatgtcgctggtgacgctctcgaagcccttgggcgagatgtccccgatgcagatgaccgctgcgccgcgcgcggcgaggaggcggcaggtggccgcgccaatgccggaggcgccgccggtgactgcaaagatgcgcgagttgacgacggtcatgatgctgtcgtgtgaggtggtgagagggggctggggtaggaaggggattgtggaaagaggtttcgtgtaagccattgaacgggcaggcggtttctggctattttaccctctccctatcctgctgttgcccctctcctctacttgagcgcggggaatgcgtcaatgatgatgcttcacgttggcgggcgaggtagcatccacccccttggctccggacctgcggtcggtggttgcgaggagcattttagtctgcggttgacagacgtgaggatgcccgagatctgcagctgccacttgctatgcaatgcagagcagcctggggcgtcgccagcacagaaggcctgatcctcgattgcaatagcggagtagggacgatagagagcagaccgccggtgctcattccgtggatgcagtgatggtatgttgcatcctcgatggcctttttttccattcaaggaatcgtgcacggggcgactttgttgtcgcatgcaatgtacgaacacgtattgcacagcagccgtcgctcattattcgaggtccgatcgatctgaagcaagctgcaaggagacgcatggggccagacagtgggccatatcgcataccaagctgtgtgtctagcagactgtcagtgattgatcaaatgatcccgccacgagagaggaacaaagacagctcgagtagtgtaagagggggggagggaggaagtaaggggaagtagggttaatgccgcaagggggggattgcccctccatcacgtgactagttgtgctggcagcccgacccaaaaagggcaatgatactcacagtgcatgtccatgtcctctgctcagctattttgagtatgtgcatggctgatgcagtgcagcatgcataaatccctcacccacctgactgaaaatcggcctcgtctacaccttcagagcaaatggcacgatcaggcacaatgccatggtctggatggattcccggtgcgcatgccagcgtctgtctcccttgctgccgtactgggcttgtgatacccaaaatgccgcacgggtagccactcatggcctctctccagcttgacaggctgtcgaaatgtgcggtgctcggcttcagacgggggaggaggggcaattgacggccgtgacagatgtatggagtgaaagacatagttccatagacatatgccggtatatatatccagaagaggaccttggtgagacctcttcatccttagaaccttcgatcctaaactacgctcccctccccggcaaagaaaacactcaaaatgaccatcaccgagacctccaagccgacgctgcgccgcatcccccggagcgccggcgacgaggccatcttccaagtcctgcaggaagacggagtcgtcgtgatcgaaggattcatgagcgcggaccaggtccgccgcttcaacggggagatcgacccgcacatgaagcagtgggaactgggccagaagtcgtaccaggagagctacctggcggggatgcgacagctgagcagcctgccgctgttcagcaagctcttccgggacgagctgatgaacgacgagctcctgcacgggctgtgcaagcggctgttcgggcccgagtccggcgactactggctgacgacctcctcggtgctggagacagagccgggctaccacgggcaggagctgcaccgcgagcacgacgggatccccatctgcaccaccctgggccgccagagccccgagtccatgctcaacttcctcaccgcgctgaccgacttcaccgcggagaacggcgccacccgcgtcctgccgggcagtcacctctgggaggacttcagcgccccgccccccaaggccgacaccgccatccccgccgtcatgaaccccggcgatgccgttctcttcaccggcaagaccctgcacggcgccggcaagaacaacaccaccgacttcctgcgccgcggcttccccctgatcatgcagtcgtgccagttcacccctgtcgaggcctcggtggccctgccccgtgagctggtcgagaccatgacccccctggcccagaagatggtcggctggcggaccgtctccgctaagggggtggatatctggacgtatgatctgaaggatctggcgacaggaattgatctcaagtcgaaccaggtggcgaaaaaggcgtgagtgggagacttgaccgcaaaataggaaccagtccgttgtcttaggtcagccaaggaaatgtgaaaggaaatctatatcataaatttgttccaaccatttgttccagactgtagacaagaaacaagattgttcgcgagtatgccgtgtgaaccaacatagtacaacttataccctgcctgctacagccttagcatgccattcacaaaatgaatacccttaggaaagtgcgccgaccgcaaatcaccctatgcaaagcgccatgtccaatcccccaatcccttaaggatgataatagatcgcccagttactgagcacaaagtatttgtgattcttattatgaaagctagatctacgaactggcttataacgttctagctggttattaaattcattacattgatctactagaagtggtaaatagctcttttatataataacctgcctgtaatatataagcacaagatatatatatacctcaacatattctagtgaattattaatattttgaatttgaaaggcaaggagctttactaactagtatagctagcttttaaataacttagatactatactttggttagaataagatatatgagaaataagtattttatgtttcatatatttggataatacttcaagcactaattctatgcagtagttgtatattcctttggtataacaataaattatttaaaagtccataatcgcgaagacataacatctacaaaattaaagtaaatcttcttgtggttgaggttcacaaatatattaaagtcaaagattgttcatttcacggagacatctagccaagtcctacaccatgcacccgaaggtcaaagtctacaccagcaagcactggggaggtgaatttaagtaacttaacttcaatggttctactccaactggattaagcccaagctaaatggctgtctcatgaggctatcattactacatacaatcactggcaaacggcatgcagagccgggcgtgtgaccctcgccggtcgattgctctttttcatctagttcccctacctacatacaaagcacttgcaactccccggcgggaagctggcaaatccggtccaacgagatgatttcctagtactgggtgatgacgccgggctccgatccatccggggtgagtgggagtttcgtctcgcgaacggtggtcccggcggtcttggtcagcttggacagctcttggacggtggctgagaggccggggtccacatggacagagttgaaggatgctgcgcgaacaggtcagatcatcagggatgtatccacggtggggaagtgtgcaggaaaggaacacggaaggggagagcttacaagtgacccagtcgcccgtctccagcgactgcagatacacgctgaggtagggtttgttcttgcggtaggagaaggagatgtaggcgtgcaggtagttcagggtctcgtggtcggcgtgcgggtagtggctgcacagctcggccttgtactggcgcgccatctcgtgccagccgcggcgctcgaagaaggtggccagcgcgtcggcgatctgcatgtcgttgcggccgaaggtggtgaagtagacctgcggctccggaacggggtcgtcgtggtggatggtgtagttggccatcaggggcagctgctcgtccgggatggtcccgatgggcagatacggggccggatacgactgcaagcccgcgggcaggcggatcagctcccacagctcgcggatcatctcgagccccgccagggtcgaggcgtcggtgcgctcgccccccatggtccagaggtcctccagggcctccagggtgaccatccgctccagcaggtagatcttgatgcgggacttggtggggtcgatcaggtcgcaggagaggagacgcgggctggccgtgctgctggggccccgcgagtagacgtactgctccagtgtgtccaggggcttgcggagctccgggtactgcttggaccagcggaaggcagacccgaagaccagctcgtggatggacctgcccgtggcgagcgacttcagggccgggtagatgtaggtcttgaccacgaactcgccgtcctggagatccagcgccagtttgttctgagtccggatctgctcgcccaccaggttgttcttgtacaggtactcagattcctcagcgctgagggtgaggtcgttcttgaagtggcggaagaggtccaggtcaatcccgctctgcagcgccatcagctgttccagactgtcccagacagcgtgggtgttgaacgggtcgcgcgccgtccccgtggccgcgttgatgggctcgtaggtgtagcgcaccagtgggccggagcagttcaggctcagctcaaagggggtgccgtatcgggtcaggatgcttagccaccggggcttgtcatttgtgggataaaccccgaggaaagggatgacgtttttcttgtagatgagcaggtgcttgtactgagcatggaggttgtagttggtggactgcagcatctgagcaaacatgggcgcggtgctgtgccaccagagctcctcatcctggttggcaaagtcgagggcgacgctcagcgtgcggtaaacttcacagtcagaggcattgcccgtcttgatgccttgcccagcagtcatggttttggttttagcgagcggagggtttgaacaaatgatgttgaagtccgttccgttccgttcgtcccttgactgggttggaaactgccgctgggttgtatttataccaggaggaatgcaaaaataggtatgacgctagttgtcgagctgaggaggggggaagaagacacaaagcgttgaagaagacgctctctcgtcacgtgtatttgggatgtatctgctccgagtatctgcagataccaggtgaagttcgaggctcacggactatctccattttcaccgctgtgggaagggtggtccgagacaacaagagctggacgaagggcaggccaaaaatccgctcccaagatgctgtcgtcgca

SEQ ID NO: 34 - DNA, 9873 bp, Aspergillus japonicus SAITO IFO 4060
agggcaggccaaaaatccgctcccaagatgctgtcgtcgcattgggtggcaatcgggcgagatcggcaagagtaatgctagatgcaggaaagacatgctcgcgcaactccgagtgccttgctgctgagcagattagacgagtcggaggaatgcaattgacgtggagaatgactgctattcttatgatcttagacctacagcacacatgtacctacgtgtatgatccctccccttgcacgcccctcttacaccccctcccctcgcacccccttgcttgcctcgagtccttggctggaatatccggacagggcggtgcctcgcgaactggaggcatcctacgctatatctggttggttgcagcctctgcgcaagttgcctttgtaagtccatacgatgatgatgggtcagtcacgagggatcttgggaggcgtgaggcaacttatcctggttattctggttggggcatacctcagccggctttcagcggtggatgacgaccgccacgactgtcgatgtcggccgggggagccatgctggcccacggtcagccactggagtactctgaacgagtccatcggcggtgctctggctcatgtcaaacccatcgctcacgtctgtcaccagtctggtcaggactcgtcggcttgcgagcaggtcttgcaagagagtcttgacagcaagtggcgggcgtcgcacacgggttagctcattccacctccctgaattcatctgacacacgggctcatccatcccacacaggcgcattgcaagactgggtctgggaaggtggcgcggaatccaaccagacatgttactacctgcggtccggtccggctggatcatgccaccagggccggattcccctctactccgcggctgtcaagtcggccagcgacgtgcagaaggtcgtggacttcactcgtcaacacaatttacgattagtgatccgcaacaccggccacgatgggagcggccgatccagtggaccggactctgtcgagatccacacccaccatctgaacagcgtgcaatatcaccccaacttccgccccgctggatcttctgagcgccagtcggctccaggacagccagcggtgacagtaggcgccggaatcctgcttggggacctctacgcccgcggcgcctcggagggatggatcgtagtgggcggggagtgtcccaccgtcggggcggccggcggattcctccagggcggaggagtatcgagttggctgagctacgcgcatggattggcagtggataatgtgcttgagtatgaagttgtgacggccaaggtgcgtgcgggttcatagcttccctccctccgcgctctaaccctaactaaccctccgcaaatgtccagggagagattgtcatcgccaacgcccatcaaaaccccgatctcttctgggcactgcgcgggggaggaggaggcacctttggcgtcgtgacccaggccacgcttcaagtccaccccgacctccccgtcagcgtcgccgacgccgtggtgacgggctcccgcgccgacgcgaccttctggtcccacggcgtcgccgccctcctgcgagcgctacaattcctcaacaaccacggcacggcaggccagttcatcctccgcaacaccgacgacgacacggtgcaagccagtctgacgatgtacttctcgaacctgaccgtccccgccgtcgccgacgagcgcatggacccgctccgccgcgccctcgaacacaacggacacccctaccagctcacctcgcggttcctgccccagatcagcagcaccttccggcacaccgcggatcgctacccggaggactacggaatcctgatgggctcggtcctggtgtcgctggacctgttcaattccgcgacgggccccgcggcgctggcgcagcacttcgcgcggctcccgatgacccccgacgatctcctgttcaccagcaacctgggcgggcgagtgtcctccctcaaccgcgacccggccagcacagccatgcatcccggctggcgtgacgccgcgcagttgctcaacttcgtccgcggggtcggggccccttctctcgcggctaaggccaccgccctccacgagctgcagacggtccagatggcgaggctgtatgagattgaaccggccttccagatctcgtaccgcaacctgggagatccctcggagcgccgcagtcgggaggtctattgggggtcgaactacgcgcggttagtggaggtcaagcggaggtgggatccggagggactgttcttcagcaagctggggattgatggtgatgcatgggacgcggaaggaatgtgtcgtcagacgcgccaaggggcgtggaatgtggcggtcaaatgggtccagagcttccttgggtctttgtctgcttctgtggtgtagttcattctctcttgatcgattggggaaatgcagttcgacatgataatattgttgtcgggatctcatgtaggataggtcgcctgcgattggagcggtcagattgcaagtgaccgtacgtgcagctgctgacctgccctgccacatttctaagcttggaaggggcagcaagacaggggggttgcactcggggggggctactccttgctggcggcgggtacattcttcgccagagcatgcaggataatgtggcagtgcgagttgacaacggagggagatgacatgacaccaaatccccggtgaggggttttggggtataggtcacaagtcgcaggatgcgagtcactgcggcaaggcaagcaaacagggcggttgtgacatgtcattctccgaataagggcaatgctgactaccttacgcgctggggaagacacatctcatggaaaaaggtcccgaaaggtcccgaaagggaagttcgatttctttcttgttcgaagtctctttccccttgctgacagcaagaagtgaggacatatcaagtccgtaaccaactccaacacctcgcgcccttcccactttgaccccctgagacattccattccgacagatctcatcatctcccccccccccccccgttttcactccctgctagctactctacaatcttgccgggatgaaagactcctccgccggcgcctcactcttctgcagcagcgtctccacccctttctggattcgcgccgccagcgctggactcaccagcttccaggtctcgtagacctgcttttggatctccgcgcgcgtctgcgcgacggactcgaccatattctggaccatggtgtcctggaactcctgcccgcggaagtccggcaggacctcgtagaagtcgcgggcgaacttgtagtcgtgctcggtggtctggagccaggacttggactggatctccttgagccactcgttgtacttggggtcgggctcgcggggctcggcgaagcggacgggctgctgctgggtcagaaagcccgggtgggcgccgtagttggcgtcgaaggtgccctggccgtcgcgcgcgatggggttgaaggtgtgggcggaccggttgacggggatctgctggtggttggcgcccagccggtaccgctgggcgtccgggtaggcgaagatgcgcgcctggaggatcgggtcctccgagggggccaccccgggcaccatgttcgagggggagtaggccagctgctcgatctcggcgaagtagttggccgggttgcgcttgagcgtcagcttgccgaagaccttgggcgggatcagcgtcacgtccttggggtacgtgcccaggttccagtgcttggtcacgtccaggatgttgtaccccaggttcggcgcgtccttggggtcgaccacctgcacgctcgccgtccacgtcggatactcgccgcgctcgatggcctcgtacaggtcgcgggtggcgtggtccggatccaggtcgttcgtgcccttggcctgctggccgtcggtgaagttgggcccgcgatcggaggagaggaagatgtggacgtatttgaacgacccgtcgggcttcacccatttgtacgcgtgggccatgtagccgctcatggaccgccagttgaacatggtcccgaagtcgctgaactgcagcaggaccatgtgcagcgactcgtggttgttggtcacccagtcccagtacatgttggggttcagcaggttggtctgcggatcccggcgctgggcgtgcatcaggctggggaacttgacggggtcgcggatgaagaagaaggggaagttgagcatcacccagtcccagttgccctcggtggtgtagaacttgcaggccatgcctttcaggtcgcgcattccctcgttggacccgcgttcgaggccagtcgtcgagaaccgcgtcacacagggggtcttcttgcccactcccaccagcatgtcgatatcgcagatatccgagatgtcggccgtcacctggaatggacggtccgggtcagcaactcagcttgggcgtggttccttccctcgtctgtaccaggatatgctgcctcatacctcaaattcgccataggctcccgctcctttagcatgcaccactcgctcggggatcttctcccggttgaagtgggcgagggtatcgaccagatggtggtcgctgagtgccagcgtccttccatccgccggaggaccctcaggcttgtggaagggacagccatttgccgtggtgtaggtcggcttgctatcgggcgccatgatttctgtgtgacaggtcttcgcttgatatcctcgctagatagcaagatgtaaacgggaacaagaaagaagacagggcaagtggtggtggaaatcaagtgcgtctggcgccgtagtcggtctccaccagacacagtaaatgtatttcaacacacatctggtcaccggtcgaggtagctgacgttgaccttgcagtccgcttcatcttcatcccttgcgtctgaagttgcctctccccaatttgttttccgaccctgtcgtcgtttcagatcccttgcatcttacacccaagcgagtccttgccgccgtccacgcccccactcattctcgctttgccacttgcaagggtcagatgcggtcatcaagctgcggaaggaagatgaagggctagcccctgctgtatgcgattgatgcatacaagcttgcctccaggggagacaaccaagggggggggaagggcagcgtagaccgcctctttgtcttcactcgcagagaccagtctctattccactttcaagactgtctgtgacaaggctaatcctacgagcctgtcatcccggttccgccctcgacagcattcggcctagacctcggaatataacatggctgccgggctggaaatggtgaacaccccgccaccgtgaccgtgaccgagaaaagaaaccctcaacatgggcagcatcaatcccccgattttggacatccgccagtcgaccttcgagacctccatcccccaacaagtaaccgagggactcaccaaagaccccaaaaccctgccggcccttcttttctacagcggcgatggcatccagcactggacgcgccactcgtgcgccccggatttctacccccgccgcgaggagatcaacatcctccacagcgaagcggctcacatggcggcctcgataagccaggacagtgttgtggtggatctgggcagcgcgtatgcaagcacacacaatctacaatcaaagcccagcagaggtgtcggggaaatgaataactaaaccgcaaacccaatgcaccttatagatccctcgataaagtcctccccttcctgcacgccctcgaggcccaacagaagcgcgttcactactacgccctcgacctgagctacccggtgctcgcctcgacgctgtccgagatccccgtggcgcagttccagtatgtccagatcgccgcgctgcacggcaccttcgaggacgggctgcagtggctgcacgactcgcccgaggtccgcgagcgcccccaccacctgctgctgttcggcctcacgatcggcaacttctcacggcccaacgccgccgcctttttacggaatatcgccgaccgggcgctggcggcgtcccccgcggagagctccatcctcctcaccctggacagctgcaagatgccgaccaaggtgctgcgcgcgtacacggcggaaggggtggtgcctttcgcgctgacctcgttgagctatgccaatcagctcttctggccgggtggggagggaagggtgtttgatgaagaggaatggtatttccacagtgagtggaacttcgcgctggggcgccacgaggcggcgctcatcccccgggatcgcgatatcaccctcgggccgccgttggagcaggtcgtcgtgcgccggggtgagaaggtgaggtttggctgcagctacaagtatagtgctgcggagcgggagcatttgttcgctggggcggggttggagaatgtggcttcctggtcggccaagggctgtgatgttgctttttatcagttgaagatgcggcccgagtgagggcttgacgtggtcatttccgggtgggattgaacagggttttggtgcagagtacaccttgttactaggaagtttcgtcaggaactgcagtttacccgttcgtatttaatctatgagagcacattaagcgggaaggagattggtattcgacaacaacaacattgtgtctttctttttgtatttggattggaacacatatccggtagccacactgcgctgacggccatcacaacaggaaaatgttgttgcacagtggtggttttcattaatctaccttagtaggaggtattgggacactcttgacatcgcttcgagaccaatattcattgaagatcaagcaacggagcagcttgaaatctcatgaacagatggtgattaagagaaaaaacaagcaaaagcaaaaacatacaacagaagggattcgctggtggtcacccacccaactactaacctcccggcgtgtggcttaagtacggctgagcggacgggaagccctgttttccacaccctgtggtcgtatgtgcttgttttaacagcactttaatttatatacaggttttcttcagatcagtactatgtttagaacacatacaaattgtatcaaggaaaagcgagaggaggaggaccagacttatgtgtggcagccgcaatcttagaaatctttctgatgaatgaggtctgaatatggtgaggctagggctacttgtgttctgataactacctaactacataatctccgagatcttccctgggtacccggcaggtacctaggtatattgttatgctgaacacgccaatagacacctgtacaagcaactggaggtgatgtaaacactcataccagtgggctcacccactcgctcgtatacgattaacaatcaggtcaatagaataagctatccacatacgccgacgcctcctttcccttgacaacccaccgttcccaggagagctcatctctagccatctcccactccggcaagacctcttcatccttctcaccggggtacctgaagcaatactcatagccctggccgtcagccctaagctgacgagcatactggccatcacgcagcccagccggacgatatcccggcacgtgcttcagtcgaaaccccaccgtgggatggatgcactcgcaagtatgcccgagaacctcattcgtgcggacatcccgaaaatagcgacgcggcctgcgcgccagcgacccgttcaggcggtaggtgaagtcgaagctgtcgggcaagtcgccggtggcccagccgtactgacggatctggcgataggccgggggagcatcgcccgacggggtgagcgggtgttgcacggcggtaatcagaccctgccctgtgtgccacagccactgggcccaggactcggactgccggcgacgttctttcttctggtacgcctccagcgcctcgttgatccgccgcagacgatcctctcgagtcttttcctcgagctggaggagcgtgcggtcgagggagacgtacccctggatctggtccagcatccacgcgaagacgatgtgggacatctcttccatgttgcccttgttcttgagggtctggttggatccgcccccgatgtgaatgtggaaccccggaaaccagacctgcgtcagctgggacggctccttctggctgtcctcccattccaaaagctcacgggcagccgcgttgcgtcgtttctcggctgcgtgtaactcctcggtgctggcggcaccgcttcttacgagttttagccactcgcgggtggcgttccgtgcctcctgcttgatcgctcgcgattggctctccgtgatattcttcggcgccgggacgaagaacatcgccgggcggaacagtcggcggtgctcgtccaacgccagcgcctggaatgcattcttgatgtctgggtttgggatcagtatcgtctcatcctgagccaatgatagcctggagggattgcttactaggcgaaagaccgacattgtgccattcaggcttatcagtccaggtcagccaggcagggagctcatggccgaacagctcggggaagcccagattgccgacggtatcgaagaccccgaccacttgcacctgccgtgactcgggggtcgatgcccattctgcggccgactcccaaaaaccctcctcttcttctgccccatcctcctcctcttccccctcttctccgccgaaggaggatcggcgcccccggaaaaactcatagtacgcatcactgccatagaaccgctccccgggcggcgtcttcttatacaccttccacagcgccgggaaattcgcgatctggtgcggcgggcagatgccgatgtccgagatgaggccggcgatggcgcgcgcggtgtaggcgccgcgcgagaacccaaagcacaggatctggtcgcccgggttccagttcagggcgcagaagtggtaggcctcgacgatgttgccctccagcccttggcccagggcgccctcggtgaagttgcgcagcgggcccgccgccgtggtgttggtgcccaccccggagtcgtaccagacgatctgctgccagggctggtggtcgtgcgggtcgatccccacgctgtcgatggcgcggcagagccgcgtcacgttcgaggggatgcgctcttggccgtcgacggcggattgccaggtgccgtcgcagcagatgaggaggcgcttgcgcgcgggttggggcttgggggtgggggtgagggggcggtgcatggtgttctctctttgaatggttaagagcgaagattaatagattatttgagatctccaaaggagttatctaccttctctttcctctagaagggtcgaggggaatcagctgggcttatcattcatgtactgtacattcatgtggtgtccgtccgtcgctacaaattgcctgctcggcagaaatgaccatcttttgttctccgtgtcaggagctgatgacccttttgcatctcagctgaactgttctttgcgagttgattggtagtcatttccttcgagtcttcctcttcagctccggctggataccccaagcgttcgtggcgcattttcagctcaaaccacgtccgtgtcattggagccctaattccaccgcaccccgcacccccggatattggaaagagctcaagaacaacactatttcttagaaaattggggaccagcaaatttgtcgttctccatattccccactcccctttgttcgtggctatataaggctgacaaacatgattgtcgtacaagttcataacaacctactagcatcaagcacgatatatctcagtattcttccatcacttccgcagtaaccatgtcagtcaaacagcatcccatacccaccagacgagagccatagagtatacgcaaggccaattctcactgcaagcacgaagcctcagaagacgaacgaaagaacccaccaacccaacccaccagaaccccaa

SEQ ID NO: 35 - DNA, 13999 bp, Aspergillus japonicus SAITO IFO 4060
cacctgacgcgccctgtagcggcgcattaagcgcggcgggtgtggtggttacgcgcagcgtgaccgctacacttgccagcgccctagcgcccgctcctttcgctttcttcccttcctttctcgccacgttcgccggctttccccgtcaagctctaaatcgggggctccctttagggttccgatttagtgctttacggcacctcgaccccaaaaaacttgattagggtgatggttcacgtagtgggccatcgccctgatagacggtttttcgccctttgacgttggagtccacgttctttaatagtggactcttgttccaaactggaacaacactcaaccctatctcggtctattcttttgatttataagggattttgccgatttcggcctattggttaaaaaatgagctgatttaacaaaaatttaacgcgaattttaacaaaatattaacgcttacaatttccattcgccattcaggctgcgcaactgttgggaagggcgatcggtgcgggcctcttcgctattacgccagctggcgaaagggggatgtgctgcaaggcgattaagttgggtaacgccagggttttcccagtcacgacgttgtaaaacgacggccagtgaattgtaatacgactcactatagggcgaattatttaaatgggtaccgggccccccctcgaggtcgacggtatcgataagcttgatctagtgcaccgcttccacctattctacctgcttgacctatctaacctgtttaatctatttaatctattttatttgttgttttttttaagcttatctacatgatgagtccacaaggtatggagatgtagggctatcattctgccgaagggctgtgcggatgaaccatggtccgggtaaaggtttcggcgcagttcatccacgacctgctcacggcgggcatgttgagatgtctctgaacgtcgtacgtaagtttatcataccctgggaggcatggtgggcgtgtggtatctctaatgactggcaatggggaatccgccgcccgttcccaccactgataataccccgttgcggggtatctgcgcacatagctacgaattctcctctacggtacagtggcctatctcaatcgagatcgatgacctagcaagcgtatggcaccaagtctcggttttattcaccgcaggctccagagcccgcccttcaacactttgagactctaggatatactgagtactgagtgaacctttgcactgcagaccaagtgccaattcccaactcccaccaaacatcatcccgaacaaaccccaccagcgaaatcacaaccaatgctcgcgattctccctcgcgtaatccacaaaccgccgcggcggccgcccggtcaccttctcgacatcttggctgacccgctcctccgcccccgtcgccaccgccgcctccatcgtcgccagcatctccgcaaagtccgggggcaacccggccgtgtcgaccaggaactggccgaacttctcggcgggcagggtgacgtgcgtaatcttccggcccaggaggtcggtgaggatctcggccatctgttttccctccagcattagtatcatcgcattctcgtcatgagcatacccttatctgggaatcggatagccgtggtaggagatagggaaagatgggaggtggggggtggaaggtgggaagaaacaaacatcagcataggacagcagatccgggcccagaaccatgtactgctgctccttctgggtgagcggctgcgtcagggcgtggaaggcgacgcgggcgatatcatccgccgagacgaaggggatcttgcccgggcccgtggccgagtagatcttgttctccgtcttgatccagctgaggtgggggtcttcgatgtggttttctgtttcactccataattagtcctgactttgggatttgggggttaggggcagggggaaagtattcgagataggaaaacacgccaatccccttctcagagggaaacgcgaggaaacggcaagggaaaagccagacaacgcacccataaaccacgtcggccgcaacaccacatacttgaccccctctttcgagtccaggtgcgcgtgcgcctcccccatcatcggatcccccttttcggtgttgctcgcgctgacgagcgcgtaccgccgcacgccccggctgtgcgccaggtcgatgaactcgaacatcgggtgcacgaactccggggcgtgggcgcctacgaggtagatagcggtgatgggctccggggcctggtcccacgcgttggtccaggtgtctttgttcagccagtcgaagtgggcggttcggtaggtggcgatcggggccgtggaggaggtgcccacgacgaaggggatctgggcttcgtgcaggagggctgagaggcggctggcggtcttgccgcggccgccgaggacgaggatggtcattttgtcggagggggttaagaagaaggatagctgtcgagggcgaggaggtaagagccaaggtaggtttgggaggtttatttgctggcgataaggatttataccggatgtatggaaatggtgggccggagaatgagggggaggggagggggcggggtgcggtgaggtgatggcacaattgacaagcacactcaccattcaccattgacatgcaggatcgagactggagcaaatcgtgttcgatgaagattggtctgatgatttcccaactcaatgcgggagaaccttgctttggggacatttctggtgggttgcaagctacgttggtgctgcagctcgaagctcggatgcgaggatgagctgggcagaagggtggggcatgcaggatgcaggatgcatgcatttacggatgtagcgtttcagaattgggccactgcggcaatagggccacgtagtcctttattggtgtacggcggcctaaagaatgaggttgtgagaggcagaggcaagaagaaagtctgcgagaagcgaggagagagccgagttagggcttgggaggggggaggtgcagagcagagggagagagagtgtgcggccacggcggtcaatttaccgccacaaccacaagaaccaaacagtcggctggataaatatgtacaagcgatatcagtccggtcaatggcactaaaatagcccaaccttcggctcttgtccagcttcaattgtatgctgcaatagcgccccgccacccaacaacatgacgatcgacccctcctcccccggggcgcccgcctcgaagctcttccaacccatgaagataggcctatgtcacctccagcaccgcctcatcatggcccccaccacccgctaccgcgccgacagcaccgcgacgcccctccccttcgtgcaagaatactacgcccaacgcgccgccgttccggggacactcctgatcaccgaagccaccgacatctccccgcaggcggccggagaaccgcacatcccaggcctgtacagcgcaacccagatcgccgcgtggcgggggatcgtctccgccgtgcaccgccggggctgctacatcttctgccagctgtgggcgaccggacgggcggcggaccccgccctgttgaaggagaaggggttccctctcgtctcgagcagcgatgtgcccatcgagactgaatcgcagcagccgcagccgctgacgcaggccgagatcgaggggtacatcgcggacttcgtgacggcggcaagggtagcggtccacgaggtggggttcgatggggtggagctgcacggcgccaacggatacctcatcgatcagttcacgcagtggccgtgcaaccagcggacggattgctggggcgggagcatcgagaaccgcgcgcggtttgcgctggcggtcactcgcgcggtggtggatgccgtcggggcggagcgcgtcggggttaagctgtcgccctggagtcagtacgccgggatgggggtgatggctgatttggtgccgcagtttgagttcttgatcaaaaggttacgggaggagtacaatgatggtcctgggctggcgtacttgcatctggcgaattcaaggtggttggatgaggagacttcccatccggatccgcatcatgaggtcttcgtgcgcgcgtggagggggtcgggcagggatggtattgaggcgaagaaagaggcgcctccggtgatcctggccggcggctatgatgctgtctcggcgcccgaggtcgctgataaggtgttcaaggaagaggacaatgtcgccgtcgcgtttgggcgatattttatctcgacgcccgacctgccgttccgcatgcaacggggcattccattgcagaagtatgatcgcgcctcgttctatacctcgcttttgaaagaaggttatctggattatccgtacagtgcggagtatgttgcggagtttgggcagaggctgaagaagaaggaggcggcggcgacagggtcgatatgtcagggttgaatggatagggtgggatcgtctgcgtatgttgcgagagcctggcgtgaaaccttttaagcaaagtagattatagaaatagatcggctacgtagggaggcccactgtgaaaaatcatgtaacgcgagcttaatgatagagagaagcacaaaacatcgtctcccagggtgatcagccatgggtgatagatacagtcgagataaaccaaccctactacatttcgaatccgacttgaagctcaaccttggctgtcattaagatggagaacaccaagtctctagatataaacacctcctctcaagctagctaaacagcatctagcatctcaaggagtagcagccccaacattgatattggccccatacaccgggcgggaatcctcgctgagcagccagacaatcgtcctggccacatcctcaggctgaatgatagggaacccctccttgatgtacgcttccttgacctcatcgatggacttcgcctggggcatgaactgcggcagagccggggtgtcggtgatacctgtttcctcaatgttagctttcccccctcgccaaagggtcatcctcctctcctctgtccatagatctatacaataaacagcacgaaagacacttacccggcgagacactgttgatccgaatccccaggggccaggcatccatggccgcacagctggtgaagtacgcgcaggcgcccttggaggtcccgtaggcgtaggcatccggcaagtgaataaacgcggcgatgctaccgatgttgacgatggcgcggtccttgggccccggaagggccttcatcgcgcggatacctgcgcgcgtgcagtagaagaccccgtccaggttgaccttgaacacgcgctgccattcctcgtccgtctcctcgagcagggccggggcctggcgggcgcccgccggctgggcgatgcccgcgacgttggccagcccgtgcaagtccccgaaggtctgcacgatggtctgtagccaggcgtcaacctcggcggacgaggagacgtccagccgggtgcagtgcaccttggtggccgggttgagggcctggatgtcgctggtgacgctctcgaagcccttgggcgagatgtccccgatgcagatgaccgctgcgccgcgcgcggcgaggaggcggcaggtggccgcgccaatgccggaggcgccgccggtgactgcaaagatgcgcgagttgacgacggtcatgatgctgtcgtgtgaggtggtgagagggggctggggtaggaaggggattgtggaaagaggtttcgtgtaagccattgaacgggcaggcggtttctggctattttaccctctccctatcctgctgttgcccctctcctctacttgagcgcggggaatgcgtcaatgatgatgcttcacgttggcgggcgaggtagcatccacccccttggctccggacctgcggtcggtggttgcgaggagcattttagtctgcggttgacagacgtgaggatgcccgagatctgcagctgccacttgctatgcaatgcagagcagcctggggcgtcgccagcacagaaggcctgatcctcgattgcaatagcggagtagggacgatagagagcagaccgccggtgctcattccgtggatgcagtgatggtatgttgcatcctcgatggcctttttttccattcaaggaatcgtgcacggggcgactttgttgtcgcatgcaatgtacgaacacgtattgcacagcagccgtcgctcattattcgaggtccgatcgatctgaagcaagctgcaaggagacgcatggggccagacagtgggccatatcgcataccaagctgtgtgtctagcagactgtcagtgattgatcaaatgatcccgccacgagagaggaacaaagacagctcgagtagtgtaagagggggggagggaggaagtaaggggaagtagggttaatgccgcaagggggggattgcccctccatcacgtgactagttgtgctggcagcccgacccaaaaagggcaatgatactcacagtgcatgtccatgtcctctgctcagctattttgagtatgtgcatggctgatgcagtgcagcatgcataaatccctcacccacctgactgaaaatcggcctcgtctacaccttcagagcaaatggcacgatcaggcacaatgccatggtctggatggattcccggtgcgcatgccagcgtctgtctcccttgctgccgtactgggcttgtgatacccaaaatgccgcacgggtagccactcatggcctctctccagcttgacaggctgtcgaaatgtgcggtgctcggcttcagacgggggaggaggggcaattgacggccgtgacagatgtatggagtgaaagacatagttccatagacatatgccggtatatatatccagaagaggaccttggtgagacctcttcatccttagaaccttcgatcctaaactacgctcccctccccggcaaagaaaacactcaaaatgaccatcaccgagacctccaagccgacgctgcgccgcatcccccggagcgccggcgacgaggccatcttccaagtcctgcaggaagacggagtcgtcgtgatcgaaggattcatgagcgcggaccaggtccgccgcttcaacggggagatcgacccgcacatgaagcagtgggaactgggccagaagtcgtaccaggagagctacctggcggggatgcgacagctgagcagcctgccgctgttcagcaagctcttccgggacgagctgatgaacgacgagctcctgcacgggctgtgcaagcggctgttcgggcccgagtccggcgactactggctgacgacctcctcggtgctggagacagagccgggctaccacgggcaggagctgcaccgcgagcacgacgggatccccatctgcaccaccctgggccgccagagccccgagtccatgctcaacttcctcaccgcgctgaccgacttcaccgcggagaacggcgccacccgcgtcctgccgggcagtcacctctgggaggacttcagcgccccgccccccaaggccgacaccgccatccccgccgtcatgaaccccggcgatgccgttctcttcaccggcaagaccctgcacggcgccggcaagaacaacaccaccgacttcctgcgccgcggcttccccctgatcatgcagtcgtgccagttcacccctgtcgaggcctcggtggccctgccccgtgagctggtcgagaccatgacccccctggcccagaagatggtcggctggcggaccgtctccgctaagggggtggatatctggacgtatgatctgaaggatctggcgacaggaattgatctcaagtcgaaccaggtggcgaaaaaggcgtgagtgggagacttgaccgcaaaataggaaccagtccgttgtcttaggtcagccaaggaaatgtgaaaggaaatctatatcataaatttgttccaaccatttgttccagactgtagacaagaaacaagattgttcgcgagtatgccgtgtgaaccaacatagtacaacttataccctgcctgctacagccttagcatgccattcacaaaatgaatacccttaggaaagtgcgccgaccgcaaatcaccctatgcaaagcgccatgtccaatcccccaatcccttaaggatgataatagatcgcccagttactgagcacaaagtatttgtgattcttattatgaaagctagatctacgaactggcttataacgttctagctggttattaaattcattacattgatctactagaagtggtaaatagctcttttatataataacctgcctgtaatatataagcacaagatatatatatacctcaacatattctagtgaattattaatattttgaatttgaaaggcaaggagctttactaactagtatagctagcttttaaataacttagatactatactttggttagaataagatatatgagaaataagtattttatgtttcatatatttggataatacttcaagcactaattctatgcagtagttgtatattcctttggtataacaataaattatttaaaagtccataatcgcgaagacataacatctacaaaattaaagtaaatcttcttgtggttgaggttcacaaatatattaaagtcaaagattgttcatttcacggagacatctagccaagtcctacaccatgcacccgaaggtcaaagtctacaccagcaagcactggggaggtgaatttaagtaacttaacttcaatggttctactccaactggattaagcccaagctaaatggctgtctcatgaggctatcattactacatacaatcactggcaaacggcatgcagagccgggcgtgtgaccctcgccggtcgattgctctttttcatctagttcccctacctacatacaaagcacttgcaactccccggcgggaagctggcaaatccggtccaacgagatgatttcctagtactgggtgatgacgccgggctccgatccatccggggtgagtgggagtttcgtctcgcgaacggtggtcccggcggtcttggtcagcttggacagctcttggacggtggctgagaggccggggtccacatggacagagttgaaggatgctgcgcgaacaggtcagatcatcagggatgtatccacggtggggaagtgtgcaggaaaggaacacggaaggggagagcttacaagtgacccagtcgcccgtctccagcgactgcagatacacgctgaggtagggtttgttcttgcggtaggagaaggagatgtaggcgtgcaggtagttcagggtctcgtggtcggcgtgcgggtagtggctgcacagctcggccttgtactggcgcgccatctcgtgccagccgcggcgctcgaagaaggtggccagcgcgtcggcgatctgcatgtcgttgcggccgaaggtggtgaagtagacctgcggctccggaacggggtcgtcgtggtggatggtgtagttggccatcaggggcagctgctcgtccgggatggtcccgatgggcagatacggggccggatacgactgcaagcccgcgggcaggcggatcagctcccacagctcgcggatcatctcgagccccgccagggtcgaggcgtcggtgcgctcgccccccatggtccagaggtcctccagggcctccagggtgaccatccgctccagcaggtagatcttgatgcgggacttggtggggtcgatcaggtcgcaggagaggagacgcgggctggccgtgctgctggggccccgcgagtagacgtactgctccagtgtgtccaggggcttgcggagctccgggtactgcttggaccagcggaaggcagacccgaagaccagctcgtggatggacctgcccgtggcgagcgacttcagggccgggtagatgtaggtcttgaccacgaactcgccgtcctggagatccagcgccagtttgttctgagtccggatctgctcgcccaccaggttgttcttgtacaggtactcagattcctcagcgctgagggtgaggtcgttcttgaagtggcggaagaggtccaggtcaatcccgctctgcagcgccatcagctgttccagactgtcccagacagcgtgggtgttgaacgggtcgcgcgccgtccccgtggccgcgttgatgggctcgtaggtgtagcgcaccagtgggccggagcagttcaggctcagctcaaagggggtgccgtatcgggtcaggatgcttagccaccggggcttgtcatttgtgggataaaccccgaggaaagggatgacgtttttcttgtagatgagcaggtgcttgtactgagcatggaggttgtagttggtggactgcagcatctgagcaaacatgggcgcggtgctgtgccaccagagctcctcatcctggttggcaaagtcgagggcgacgctcagcgtgcggtaaacttcacagtcagaggcattgcccgtcttgatgccttgcccagcagtcatggttttggttttagcgagcggagggtttgaacaaatgatgttgaagtccgttccgttccgttcgtcccttgactgggttggaaactgccgctgggttgtatttataccaggaggaatgcaaaaataggtatgacgctagttgtcgagctgaggaggggggaagaagacacaaagcgttgaagaagacgctctctcgtcacgtgtatttgggatgtatctgctccgagtatctgcagataccaggtgaagttcgaggctcacggactatctccattttcaccgctgtgggaagggtggtccgagacaacaagagctggacgaagggcaggccaaaaatccgctcccaagatgctgtcgtcgcaatcgaattcctgcagcccgggggatccactagttctagagaataggaacttccgttaactgatattgaaggagcattttttgggcttggctggagctagtggaggtcaacaatgaatgcctattttggtttagtcgtccaggcggtgagcacaaaatttgtgtcgtttgacaagatggttcatttaggcaactggtcagatcagccccacttgtagcagtagcggcggcgctcgaagtgtgactcttattagcagacaggaacgaggacattattatcatctgctgcttggtgcacgataacttggtgcgtttgtcaagcaaggtaagtggacgacccggtcataccttcttaagttcgcccttcctccctttatttcagattcaatctgacttacctattctacctaagcattcatggccaccctcgatgacacggcttaccgctaccgtaccagcgtccccggcgacgccgaagccatcgaggccctggatggctctttcaccacggataccgtctttcgcgttaccgctaccggtgacggcttcacgctccgtgaggtgcccgtcgaccctcccctgaccaaggttttccctgatgacgaatctgacgatgaatccgacgatggcgaggacggcgatcccgactctcgcacgttcgtcgcttacggcgatgacggtgacctggccggctttgtcgttgtgtcctattccggttggaaccgtcgcctgaccgtcgaagacatcgaggttgcccccgagcatcgcggccacggtgtcggccgcgctctcatgggcctcgccacggagttcgcccgtgagcgcggcgccggtcacctctggctggaggttaccaatgtcaacgctcccgccattcacgcctaccgtcgcatgggctttaccctctgcggcctggataccgctctctacgatggcaccgcctccgatggcgagcaggccctctatatgagcatgccttgcccctgagaagttcctatactttatttaaatctagagcggccgccaccgcggtggagctccagcttttgttccctttagtgagggttaatttcgagcttggcgtaatcatggtcatagctgtttcctgtgtgaaattgttatccgctcacaattccacacaacatacgagccggaagcataaagtgtaaagcctggggtgcctaatgagtgagctaactcacattaattgcgttgcgctcactgcccgctttccagtcgggaaacctgtcgtgccagctgcattaatgaatcggccaacgcgcggggagaggcggtttgcgtattgggcgctcttccgcttcctcgctcactgactcgctgcgctcggtcgttcggctgcggcgagcggtatcagctcactcaaaggcggtaatacggttatccacagaatcaggggataacgcaggaaagaacatgtgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcatagctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaaggacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggtctgacgctcagtggaacgaaaactcacgttaagggattttggtcatgagattatcaaaaaggatcttcacctagatccttttaaattaaaaatgaagttttaaatcaatctaaagtatatatgagtaaacttggtctgacagttaccaatgcttaatcagtgaggcacctatctcagcgatctgtctatttcgttcatccatagttgcctgactccccgtcgtgtagataactacgatacgggagggcttaccatctggccccagtgctgcaatgataccgcgagacccacgctcaccggctccagatttatcagcaataaaccagccagccggaagggccgagcgcagaagtggtcctgcaactttatccgcctccatccagtctattaattgttgccgggaagctagagtaagtagttcgccagttaatagtttgcgcaacgttgttgccattgctacaggcatcgtggtgtcacgctcgtcgtttggtatggcttcattcagctccggttcccaacgatcaaggcgagttacatgatcccccatgttgtgcaaaaaagcggttagctccttcggtcctccgatcgttgtcagaagtaagttggccgcagtgttatcactcatggttatggcagcactgcataattctcttactgtcatgccatccgtaagatgcttttctgtgactggtgagtactcaaccaagtcattctgagaatagtgtatgcggcgaccgagttgctcttgcccggcgtcaatacgggataataccgcgccacatagcagaactttaaaagtgctcatcattggaaaacgttcttcggggcgaaaactctcaaggatcttaccgctgttgagatccagttcgatgtaacccactcgtgcacccaactgatcttcagcatcttttactttcaccagcgtttctgggtgagcaaaaacaggaaggcaaaatgccgcaaaaaagggaataagggcgacacggaaatgttgaatactcatactcttcctttttcaatattattgaagcatttatcagggttattgtctcatgagcggatacatatttgaatgtatttagaaaaataaacaaataggggttccgcgcacatttccccgaaaagtgc

SEQ ID NO: 36 - DNA, 12848 bp, Aspergillus japonicus SAITO IFO 4060
cacctgacgcgccctgtagcggcgcattaagcgcggcgggtgtggtggttacgcgcagcgtgaccgctacacttgccagcgccctagcgcccgctcctttcgctttcttcccttcctttctcgccacgttcgccggctttccccgtcaagctctaaatcgggggctccctttagggttccgatttagtgctttacggcacctcgaccccaaaaaacttgattagggtgatggttcacgtagtgggccatcgccctgatagacggtttttcgccctttgacgttggagtccacgttctttaatagtggactcttgttccaaactggaacaacactcaaccctatctcggtctattcttttgatttataagggattttgccgatttcggcctattggttaaaaaatgagctgatttaacaaaaatttaacgcgaattttaacaaaatattaacgcttacaatttccattcgccattcaggctgcgcaactgttgggaagggcgatcggtgcgggcctcttcgctattacgccagctggcgaaagggggatgtgctgcaaggcgattaagttgggtaacgccagggttttcccagtcacgacgttgtaaaacgacggccagtgaattgtaatacgactcactatagggcgaattatttaaatgggtaccgggccccccctcgaggtcgacggtatcgataagcttgataagggcaggccaaaaatccgctcccaagatgctgtcgtcgcattgggtggcaatcgggcgagatcggcaagagtaatgctagatgcaggaaagacatgctcgcgcaactccgagtgccttgctgctgagcagattagacgagtcggaggaatgcaattgacgtggagaatgactgctattcttatgatcttagacctacagcacacatgtacctacgtgtatgatccctccccttgcacgcccctcttacaccccctcccctcgcacccccttgcttgcctcgagtccttggctggaatatccggacagggcggtgcctcgcgaactggaggcatcctacgctatatctggttggttgcagcctctgcgcaagttgcctttgtaagtccatacgatgatgatgggtcagtcacgagggatcttgggaggcgtgaggcaacttatcctggttattctggttggggcatacctcagccggctttcagcggtggatgacgaccgccacgactgtcgatgtcggccgggggagccatgctggcccacggtcagccactggagtactctgaacgagtccatcggcggtgctctggctcatgtcaaacccatcgctcacgtctgtcaccagtctggtcaggactcgtcggcttgcgagcaggtcttgcaagagagtcttgacagcaagtggcgggcgtcgcacacgggttagctcattccacctccctgaattcatctgacacacgggctcatccatcccacacaggcgcattgcaagactgggtctgggaaggtggcgcggaatccaaccagacatgttactacctgcggtccggtccggctggatcatgccaccagggccggattcccctctactccgcggctgtcaagtcggccagcgacgtgcagaaggtcgtggacttcactcgtcaacacaatttacgattagtgatccgcaacaccggccacgatgggagcggccgatccagtggaccggactctgtcgagatccacacccaccatctgaacagcgtgcaatatcaccccaacttccgccccgctggatcttctgagcgccagtcggctccaggacagccagcggtgacagtaggcgccggaatcctgcttggggacctctacgcccgcggcgcctcggagggatggatcgtagtgggcggggagtgtcccaccgtcggggcggccggcggattcctccagggcggaggagtatcgagttggctgagctacgcgcatggattggcagtggataatgtgcttgagtatgaagttgtgacggccaaggtgcgtgcgggttcatagcttccctccctccgcgctctaaccctaactaaccctccgcaaatgtccagggagagattgtcatcgccaacgcccatcaaaaccccgatctcttctgggcactgcgcgggggaggaggaggcacctttggcgtcgtgacccaggccacgcttcaagtccaccccgacctccccgtcagcgtcgccgacgccgtggtgacgggctcccgcgccgacgcgaccttctggtcccacggcgtcgccgccctcctgcgagcgctacaattcctcaacaaccacggcacggcaggccagttcatcctccgcaacaccgacgacgacacggtgcaagccagtctgacgatgtacttctcgaacctgaccgtccccgccgtcgccgacgagcgcatggacccgctccgccgcgccctcgaacacaacggacacccctaccagctcacctcgcggttcctgccccagatcagcagcaccttccggcacaccgcggatcgctacccggaggactacggaatcctgatgggctcggtcctggtgtcgctggacctgttcaattccgcgacgggccccgcggcgctggcgcagcacttcgcgcggctcccgatgacccccgacgatctcctgttcaccagcaacctgggcgggcgagtgtcctccctcaaccgcgacccggccagcacagccatgcatcccggctggcgtgacgccgcgcagttgctcaacttcgtccgcggggtcggggccccttctctcgcggctaaggccaccgccctccacgagctgcagacggtccagatggcgaggctgtatgagattgaaccggccttccagatctcgtaccgcaacctgggagatccctcggagcgccgcagtcgggaggtctattgggggtcgaactacgcgcggttagtggaggtcaagcggaggtgggatccggagggactgttcttcagcaagctggggattgatggtgatgcatgggacgcggaaggaatgtgtcgtcagacgcgccaaggggcgtggaatgtggcggtcaaatgggtccagagcttccttgggtctttgtctgcttctgtggtgtagttcattctctcttgatcgattggggaaatgcagttcgacatgataatattgttgtcgggatctcatgtaggataggtcgcctgcgattggagcggtcagattgcaagtgaccgtacgtgcagctgctgacctgccctgccacatttctaagcttggaaggggcagcaagacaggggggttgcactcggggggggctactccttgctggcggcgggtacattcttcgccagagcatgcaggataatgtggcagtgcgagttgacaacggagggagatgacatgacaccaaatccccggtgaggggttttggggtataggtcacaagtcgcaggatgcgagtcactgcggcaaggcaagcaaacagggcggttgtgacatgtcattctccgaataagggcaatgctgactaccttacgcgctggggaagacacatctcatggaaaaaggtcccgaaaggtcccgaaagggaagttcgatttctttcttgttcgaagtctctttccccttgctgacagcaagaagtgaggacatatcaagtccgtaaccaactccaacacctcgcgcccttcccactttgaccccctgagacattccattccgacagatctcatcatctcccccccccccccccgttttcactccctgctagctactctacaatcttgccgggatgaaagactcctccgccggcgcctcactcttctgcagcagcgtctccacccctttctggattcgcgccgccagcgctggactcaccagcttccaggtctcgtagacctgcttttggatctccgcgcgcgtctgcgcgacggactcgaccatattctggaccatggtgtcctggaactcctgcccgcggaagtccggcaggacctcgtagaagtcgcgggcgaacttgtagtcgtgctcggtggtctggagccaggacttggactggatctccttgagccactcgttgtacttggggtcgggctcgcggggctcggcgaagcggacgggctgctgctgggtcagaaagcccgggtgggcgccgtagttggcgtcgaaggtgccctggccgtcgcgcgcgatggggttgaaggtgtgggcggaccggttgacggggatctgctggtggttggcgcccagccggtaccgctgggcgtccgggtaggcgaagatgcgcgcctggaggatcgggtcctccgagggggccaccccgggcaccatgttcgagggggagtaggccagctgctcgatctcggcgaagtagttggccgggttgcgcttgagcgtcagcttgccgaagaccttgggcgggatcagcgtcacgtccttggggtacgtgcccaggttccagtgcttggtcacgtccaggatgttgtaccccaggttcggcgcgtccttggggtcgaccacctgcacgctcgccgtccacgtcggatactcgccgcgctcgatggcctcgtacaggtcgcgggtggcgtggtccggatccaggtcgttcgtgcccttggcctgctggccgtcggtgaagttgggcccgcgatcggaggagaggaagatgtggacgtatttgaacgacccgtcgggcttcacccatttgtacgcgtgggccatgtagccgctcatggaccgccagttgaacatggtcccgaagtcgctgaactgcagcaggaccatgtgcagcgactcgtggttgttggtcacccagtcccagtacatgttggggttcagcaggttggtctgcggatcccggcgctgggcgtgcatcaggctggggaacttgacggggtcgcggatgaagaagaaggggaagttgagcatcacccagtcccagttgccctcggtggtgtagaacttgcaggccatgcctttcaggtcgcgcattccctcgttggacccgcgttcgaggccagtcgtcgagaaccgcgtcacacagggggtcttcttgcccactcccaccagcatgtcgatatcgcagatatccgagatgtcggccgtcacctggaatggacggtccgggtcagcaactcagcttgggcgtggttccttccctcgtctgtaccaggatatgctgcctcatacctcaaattcgccataggctcccgctcctttagcatgcaccactcgctcggggatcttctcccggttgaagtgggcgagggtatcgaccagatggtggtcgctgagtgccagcgtccttccatccgccggaggaccctcaggcttgtggaagggacagccatttgccgtggtgtaggtcggcttgctatcgggcgccatgatttctgtgtgacaggtcttcgcttgatatcctcgctagatagcaagatgtaaacgggaacaagaaagaagacagggcaagtggtggtggaaatcaagtgcgtctggcgccgtagtcggtctccaccagacacagtaaatgtatttcaacacacatctggtcaccggtcgaggtagctgacgttgaccttgcagtccgcttcatcttcatcccttgcgtctgaagttgcctctccccaatttgttttccgaccctgtcgtcgtttcagatcccttgcatcttacacccaagcgagtccttgccgccgtccacgcccccactcattctcgctttgccacttgcaagggtcagatgcggtcatcaagctgcggaaggaagatgaagggctagcccctgctgtatgcgattgatgcatacaagcttgcctccaggggagacaaccaagggggggggaagggcagcgtagaccgcctctttgtcttcactcgcagagaccagtctctattccactttcaagactgtctgtgacaaggctaatcctacgagcctgtcatcccggttccgccctcgacagcattcggcctagacctcggaatataacatggctgccgggctggaaatggtgaacaccccgccaccgtgaccgtgaccgagaaaagaaaccctcaacatgggcagcatcaatcccccgattttggacatccgccagtcgaccttcgagacctccatcccccaacaagtaaccgagggactcaccaaagaccccaaaaccctgccggcccttcttttctacagcggcgatggcatccagcactggacgcgccactcgtgcgccccggatttctacccccgccgcgaggagatcaacatcctccacagcgaagcggctcacatggcggcctcgataagccaggacagtgttgtggtggatctgggcagcgcgtatgcaagcacacacaatctacaatcaaagcccagcagaggtgtcggggaaatgaataactaaaccgcaaacccaatgcaccttatagatccctcgataaagtcctccccttcctgcacgccctcgaggcccaacagaagcgcgttcactactacgccctcgacctgagctacccggtgctcgcctcgacgctgtccgagatccccgtggcgcagttccagtatgtccagatcgccgcgctgcacggcaccttcgaggacgggctgcagtggctgcacgactcgcccgaggtccgcgagcgcccccaccacctgctgctgttcggcctcacgatcggcaacttctcacggcccaacgccgccgcctttttacggaatatcgccgaccgggcgctggcggcgtcccccgcggagagctccatcctcctcaccctggacagctgcaagatgccgaccaaggtgctgcgcgcgtacacggcggaaggggtggtgcctttcgcgctgacctcgttgagctatgccaatcagctcttctggccgggtggggagggaagggtgtttgatgaagaggaatggtatttccacagtgagtggaacttcgcgctggggcgccacgaggcggcgctcatcccccgggatcgcgatatcaccctcgggccgccgttggagcaggtcgtcgtgcgccggggtgagaaggtgaggtttggctgcagctacaagtatagtgctgcggagcgggagcatttgttcgctggggcggggttggagaatgtggcttcctggtcggccaagggctgtgatgttgctttttatcagttgaagatgcggcccgagtgagggcttgacgtggtcatttccgggtgggattgaacagggttttggtgcagagtacaccttgttactaggaagtttcgtcaggaactgcagtttacccgttcgtatttaatctatgagagcacattaagcgggaaggagattggtattcgacaacaacaacattgtgtctttctttttgtatttggattggaacacatatccggtagccacactgcgctgacggccatcacaacaggaaaatgttgttgcacagtggtggttttcattaatctaccttagtaggaggtattgggacactcttgacatcgcttcgagaccaatattcattgaagatcaagcaacggagcagcttgaaatctcatgaacagatggtgattaagagaaaaaacaagcaaaagcaaaaacatacaacagaagggattcgctggtggtcacccacccaactactaacctcccggcgtgtggcttaagtacggctgagcggacgggaagccctgttttccacaccctgtggtcgtatgtgcttgttttaacagcactttaatttatatacaggttttcttcagatcagtactatgtttagaacacatacaaattgtatcaaggaaaagcgagaggaggaggaccagacttatgtgtggcagccgcaatcttagaaatctttctgatgaatgaggtctgaatatggtgaggctagggctacttgtgttctgataactacctaactacataatctccgagatcttccctgggtacccggcaggtacctaggtatattgttatgctgaacacgccaatagacacctgtacaagcaactggaggtgatgtaaacactcataccagtgggctcacccactcgctcgtatacgattaacaatcaggtcaatagaataagctatccacatacgccgacgcctcctttcccttgacaacccaccgttcccaggagagctcatctctagccatctcccactccggcaagacctcttcatccttctcaccggggtacctgaagcaatactcatagccctggccgtcagccctaagctgacgagcatactggccatcacgcagcccagccggacgatatcccggcacgtgcttcagtcgaaaccccaccgtgggatggatgcactcgcaagtatgcccgagaacctcattcgtgcggacatcccgaaaatagcgacgcggcctgcgcgccagcgacccgttcaggcggtaggtgaagtcgaagctgtcgggcaagtcgccggtggcccagccgtactgacggatctggcgataggccgggggagcatcgcccgacggggtgagcgggtgttgcacggcggtaatcagaccctgccctgtgtgccacagccactgggcccaggactcggactgccggcgacgttctttcttctggtacgcctccagcgcctcgttgatccgccgcagacgatcctctcgagtcttttcctcgagctggaggagcgtgcggtcgagggagacgtacccctggatctggtccagcatccacgcgaagacgatgtgggacatctcttccatgttgcccttgttcttgagggtctggttggatccgcccccgatgtgaatgtggaaccccggaaaccagacctgcgtcagctgggacggctccttctggctgtcctcccattccaaaagctcacgggcagccgcgttgcgtcgtttctcggctgcgtgtaactcctcggtgctggcggcaccgcttcttacgagttttagccactcgcgggtggcgttccgtgcctcctgcttgatcgctcgcgattggctctccgtgatattcttcggcgccgggacgaagaacatcgccgggcggaacagtcggcggtgctcgtccaacgccagcgcctggaatgcattcttgatgtctgggtttgggatcagtatcgtctcatcctgagccaatgatagcctggagggattgcttactaggcgaaagaccgacattgtgccattcaggcttatcagtccaggtcagccaggcagggagctcatggccgaacagctcggggaagcccagattgccgacggtatcgaagaccccgaccacttgcacctgccgtgactcgggggtcgatgcccattctgcggccgactcccaaaaaccctcctcttcttctgccccatcctcctcctcttccccctcttctccgccgaaggaggatcggcgcccccggaaaaactcatagtacgcatcactgccatagaaccgctccccgggcggcgtcttcttatacaccttccacagcgccgggaaattcgcgatctggtgcggcgggcagatgccgatgtccgagatgaggccggcgatggcgcgcgcggtgtaggcgccgcgcgagaacccaaagcacaggatctggtcgcccgggttccagttcagggcgcagaagtggtaggcctcgacgatgttgccctccagcccttggcccagggcgccctcggtgaagttgcgcagcgggcccgccgccgtggtgttggtgcccaccccggagtcgtaccagacgatctgctgccagggctggtggtcgtgcgggtcgatccccacgctgtcgatggcgcggcagagccgcgtcacgttcgaggggatgcgctcttggccgtcgacggcggattgccaggtgccgtcgcagcagatgaggaggcgcttgcgcgcgggttggggcttgggggtgggggtgagggggcggtgcatggtgttctctctttgaatggttaagagcgaagattaatagattatttgagatctccaaaggagttatctaccttctctttcctctagaagggtcgaggggaatcagctgggcttatcattcatgtactgtacattcatgtggtgtccgtccgtcgctacaaattgcctgctcggcagaaatgaccatcttttgttctccgtgtcaggagctgatgacccttttgcatctcagctgaactgttctttgcgagttgattggtagtcatttccttcgagtcttcctcttcagctccggctggataccccaagcgttcgtggcgcattttcagctcaaaccacgtccgtgtcattggagccctaattccaccgcaccccgcacccccggatattggaaagagctcaagaacaacactatttcttagaaaattggggaccagcaaatttgtcgttctccatattccccactcccctttgttcgtggctatataaggctgacaaacatgattgtcgtacaagttcataacaacctactagcatcaagcacgatatatctcagtattcttccatcacttccgcagtaaccatgtcagtcaaacagcatcccatacccaccagacgagagccatagagtatacgcaaggccaattctcactgcaagcacgaagcctcagaagacgaacgaaagaacccaccaacccaacccaccagaaccccaaatcgaattcctgcagcccgggggatccactagttctagaatttaaatgcggccgccaccgcggtggagctccagcttttgttccctttagtgagggttaatttcgagcttggcgtaatcatggtcatagctgtttcctgtgtgaaattgttatccgctcacaattccacacaacatacgagccggaagcataaagtgtaaagcctggggtgcctaatgagtgagctaactcacattaattgcgttgcgctcactgcccgctttccagtcgggaaacctgtcgtgccagctgcattaatgaatcggccaacgcgcggggagaggcggtttgcgtattgggcgctcttccgcttcctcgctcactgactcgctgcgctcggtcgttcggctgcggcgagcggtatcagctcactcaaaggcggtaatacggttatccacagaatcaggggataacgcaggaaagaacatgtgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcatagctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaaggacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggtctgacgctcagtggaacgaaaactcacgttaagggattttggtcatgagattatcaaaaaggatcttcacctagatccttttaaattaaaaatgaagttttaaatcaatctaaagtatatatgagtaaacttggtctgacagttaccaatgcttaatcagtgaggcacctatctcagcgatctgtctatttcgttcatccatagttgcctgactccccgtcgtgtagataactacgatacgggagggcttaccatctggccccagtgctgcaatgataccgcgagacccacgctcaccggctccagatttatcagcaataaaccagccagccggaagggccgagcgcagaagtggtcctgcaactttatccgcctccatccagtctattaattgttgccgggaagctagagtaagtagttcgccagttaatagtttgcgcaacgttgttgccattgctacaggcatcgtggtgtcacgctcgtcgtttggtatggcttcattcagctccggttcccaacgatcaaggcgagttacatgatcccccatgttgtgcaaaaaagcggttagctccttcggtcctccgatcgttgtcagaagtaagttggccgcagtgttatcactcatggttatggcagcactgcataattctcttactgtcatgccatccgtaagatgcttttctgtgactggtgagtactcaaccaagtcattctgagaatagtgtatgcggcgaccgagttgctcttgcccggcgtcaatacgggataataccgcgccacatagcagaactttaaaagtgctcatcattggaaaacgttcttcggggcgaaaactctcaaggatcttaccgctgttgagatccagttcgatgtaacccactcgtgcacccaactgatcttcagcatcttttactttcaccagcgtttctgggtgagcaaaaacaggaaggcaaaatgccgcaaaaaagggaataagggcgacacggaaatgttgaatactcatactcttcctttttcaatattattgaagcatttatcagggttattgtctcatgagcggatacatatttgaatgtatttagaaaaataaacaaataggggttccgcgcacatttccccgaaaagtgc

SEQ ID NO: 37 - DNA, 30 bp, Artificial Sequence
  P1_5primer
ctagtgcaccgcttccacctattctacctg

SEQ ID NO: 38 - DNA, 30 bp, Artificial Sequence
  P2_3primer
tctcggaccacccttcccacagcggtgaaa

SEQ ID NO: 39 - DNA, 33 bp, Artificial Sequence
  P3_5`primer
aagggcaggccaaaaatccgctcccaagatgct

SEQ ID NO: 40 - DNA, 33 bp, Artificial Sequence
  P4_3primer
ttggggttctggtgggttgggttggtgggttct